(19) United States
(12) Patent Application Publication
(10) Pub. No.: US 2026/0256733 A1
(43) Pub. Date: Sep. 3, 2026

(54) COMPOSITIONS AND METHODS FOR MODULATING STRESS AND USES THEREOF

(71) Applicant: BRIGHTSEED, INC., South San Francisco, CA (US)

(72) Inventors: Nicholas WOODWARD, South San Francisco, CA (US); Chuan WANG, South San Francisco, CA (US); Ilan Edmond CHEMMAMA, South San Francisco, CA (US); Pranatchareeya CHANKHAMJON, South San Francisco, CA (US); Meng XU, South San Francisco, CA (US); Lee Heil CHAE, South San Francisco, CA (US)

(73) Assignee: BRIGHTSEED, INC., South San Francisco, CA (US)

(21) Appl. No.: 19/163,837

(22) PCT Filed: Mar. 8, 2024
(86) PCT No.: PCT/US2024/019228

§ 371 Date: Sep. 9, 2025

Related U.S. Application Data
(60) Provisional application No. 63/489,625, filed on Mar. 10, 2023.
(60) Provisional application No. 63/508,827, filed on Jun. 16, 2023.

(51) Int. Cl. A61K 31/353; A61K 36/482

(52) U.S. Cl. CPC A61K 31/353; A61K 36/482

(57) ABSTRACT

Disclosed herein are compositions and methods for treating, preventing, reducing, modulating, or ameliorating stress, symptoms of stress, or deleterious effects of chronic stress, by providing the composition and a carrier. The methods may include, for example, administering a therapeutically effective compound of a plant extract. The compositions and methods may be capable of modulating mGlu2 and/or mGlu3 receptors.

### CROSS-REFERENCE TO RELATED APPLICATIONS

This application claims the benefit of U.S. Provisional Application No. 63/508,827, filed Jun. 16, 2023, and U.S. Provisional Application No. 63/489,625, filed Mar. 10, 2023, which are incorporated by reference in their entirety.

### FIELD OF THE DISCLOSURE

The present disclosure relates to compounds and compositions for modulating stress and methods for using the same. More particularly, the present disclosure relates to compositions and methods for modulating stress by administering an effective amount of the composition and a carrier.

### BACKGROUND

Chronic stress puts health at risk. The long-term activation of the stress response system and the overexposure to cortisol and other stress hormones that come with it can disrupt almost all human biological processes. This can put the body at increased risk for a variety of physical and mental health problems, including anxiety, depression, digestive issues, headaches, muscle tension and pain, heart disease, high blood pressure, stroke, sleep problems, weight gain, cancer, and memory and concentration impairment. Chronic stress also suppresses the body's immune system, making it harder to recover from illnesses. Furthermore, it has been estimated that 60% to 80% of primary care doctor visits are related to stress, yet only 3% of patients receive stress management help.

There are three main approaches to chronic stress treatment: psychotherapy, lifestyle changes, and therapeutic intervention. While psychotherapy and lifestyle changes can be impactful in minimizing chronic stress and its symptoms, both require significant investment in time and effort. Therapeutic interventions are often anti-anxiety and anti-depression medicines, including SSRIs (selective serotonin reuptake inhibitors), CNS depressants, BuSpar, and Beta blockers. However, most therapeutic solutions and combinations are unfortunately ineffective for many patients. In addition, most currently approved drugs require weeks to months of daily administration to obtain maximum efficacy, placing a considerable personal, psychosocial, and professional burden on affected individuals and the healthcare system.

Furthermore, most therapeutic solutions are not suitable for daily consumption as a nutritional intervention or prevention. Traditional herbal remedies, including dietary supplements, are marketed as stress relief aids. Some examples are kava, passionflower, valerian, chamomile, lavender, and lemon balm. These products are not regulated, and the purity of ingredients and clinical response can vary. There is a lack of evidence supporting these herbal remedies.

Clearly, there remains an unmet need to develop a fast-acting and safe nutritional intervention offering for the improvement of stress and stress related conditions.

### SUMMARY OF THE DISCLOSURE

Some embodiments of the disclosure relate to an oral composition for treating, reducing, modulating, or ameliorating stress, symptoms of stress, or deleterious effects of chronic stress. The oral composition includes a therapeutically effective amount of a plant extract, and one or more carriers or excipients, wherein, the therapeutically effective amount of the plant extract is capable of treating, reducing, modulating, or ameliorating stress, symptoms of stress, or deleterious effects of chronic stress. In some embodiments, the plant extract is selected from the group consisting of Rheum officinale, Phyllanthus emblica, Malus domestica, Tripterygium wilfordii, Aloe barbadensis, Theobroma cacao, Chaenomeles sinensis, Hypericum perforatum, Citrus unshiu, Nelumbo nucifera, Acacia luederitzii, Cassia abbreviate, Cassia fistula, Colophospermum mopane, Julbernardia globiflora, Habranthus brachyandrus, Monactis macbridei, Arnica acaulis, Arnica montana, Daucus carota, Bauhinia manca, Botrytis cinerea, Brosimum acutifolium, Broussonetia kazinoki, Broussonetia papyrifera, Dalbergia latifolia, Dracaena cambodiana, Dracaena draco, Narcissus pseudonarcissus, Phanera guianensis, Rohdea chinensis, Soymida febrifuga, Taxus brevifolia, Tupistra chinensis, Abri herba, Abrus pulchellus subsp. Cantoniensis, Achyranthes bidentata, Aconitum seravschanicum, Aconitum tasiromontanum, Acori tataninowii rhizome, Acorus gramineus, Acorus tatarinowii, Actinidia chinensis planch, Adansonia grandidieri, Aframomum giganteum, Aizopsis selkiana, Albizia myriophylla, Alisma plantago-aquatica subsp. orientale, Aloe castellorum, Aloe vera, Alpiniae officinarum rhizome, Amaranthus hypochondriacus, Ampelopsis japonica, Amphorogyne spicata, Anaphalis lacteal, Anchusa azurea, Andrographis laxiflora, Andrographis neesiana, Anemarrhena asphodeloides, Aphyllocladus denticulatus, Aralia decaisneana, Artemisia annua, Artemisia parviflora, Artemisia scoparia [Syn. Artemisia capillaris v, Artemisia scoparia, Aspidosperma discolor, Asteris radix et rhizome, Aster sedifolius, Aster tataricus, Atadinus libanoticus, Baccharis potosina, Balanophora laxiflora, Bassia muricata, Benincasa hispida, Beta vulgaris vulgaris, Bistorta elliptica, Bistorta officinalis, Boehmeriae rhizoma et radix, Boehmeria nivea, Boehmeria platanifolia [Syn. Boehmeria tricuspis, Boehmeria platanifolia, Bolbostemae paniculati bulbus, Bolbostemma paniculatum, Brassica rapa, Bridelia stipularis, Canarium bengalense, Carduus tenuiflorus, Cascara sagrada, Cassia alata L., Cassia angolensis, Cassiae semen, Cassia fastuosa, Cassia javanica, Cassia obtusifolia, Cassia occidentalis, Cassia speciosa, Cassia tora L., Cassia tora, Cassia torosa L., Catalpa longissima, Caulerpa peltata, Caulis Polygoni Multiflori, Caulis sargentodoxae, Caulocystis cephalornithos, Cephaelis ipecacuanha, Cephalotaxus griffithii, Chaiturus marrubiastrum, Chamaecrista mimosoides, Chamaecrista nigricans, Chamaecrista nomame, Chamaecrista pumila, Cheilanthes argentea, Chenopodium quinoa, Cichorium intybus, Cirsium oligophyllum, Clibadium sprucei, Coreopsis tinctoria, Corydalis rhizoma, Cranfillia fluviatilis, Cratoxylum formosum, Cremastra appendiculata, Cynanchum viminale, Dalbergia hupeana, Dalbergia lanceolaria subsp. paniculata, Degenia velebitica, Delphinium nuttallianum, Delphinium virgatum, Dendriopoterium menendezii, Dendrobium thyrsiflorum, Derris canarensis, Derris cuneifolia, Derris oblonga, Dianthi herba, Dianthus caryophyllus, Dictyopteris australis, Dillenia indica, Dioscoreae bulbiferae, Dipsacus azureus, Dodonaea viscosa, Dysoxylum cumingianum, Dysoxylum muelleri, Elymus repens, Enceliopsis argophylla, Endophytic aspergillus, Epimedium koreanum, Epimrdii herba, Eria mollis, Eriocaulon buergerianum, Eriocaulon sieboldianum, Erythrina lysistemon, Erythrophleum ivorense, Euodia bodinieri, Euodia officinalis, Euodia ruticarpa, Fagopyrum esculentum, Fagopyrum tataricum, Fallopia multiflora, Fallopia sachalinensis, Flacourtia jangomas, Frangula alnus, Frangula purshiana, Fridericia triplinervia, Garcinia polyantha, Gleichenia quadripartita, Glycyrrhiza uralensis, Gnaphalii affinis herba, Grangea maderaspatana, Hedera helix, Helichrysum chionosphaerum, Helichrysum plicatum, Heynea trijuga, Hoveniae dulcis semen, Hymenoxys lemmonii, Hypericum prolificum, Hypericum sampsonii, Isatdis radix, Isatis tinctoria, Jacquemontia paniculate, Juniperus formosana, Justicia secunda, Lactuca plumieri, Lactuca sativa, Lanaria lanata, Larix kaempferior, Lavandula vera, Leptocereus quadricostatus, Lespedeza tomentosa, Ligularia pleurocaulis, Lilii bulbus, Lilium brownii f. e. brown var. colchesteri wils, Lilium brownii var. viridulum [Syn. Lilium brown, Lilium brownii var. viridulum, Lobelia berlandieri, Loranthus pentandrus, Lupinus corymbosus, Lycii cortex, Maclura tinctoria, Magnolia figo, Magnolia ovata, Mandragora autumnalis, Melicope triphylla, Mentha arvensis, Mentha canadensis, Menthae herba, Metrodorea nigra, Miliusa velutina, Mitracarpus scaber, Muehlenbeckia tamnifolia, Muntingia calabura, Myrsine africana, Nekemias grossedentata, Neobalanocarpus heimii, Nepeta granatensis, Nepeta parnassica, Nuxia floribunda, Onobrychis arenaria, Oryctes monoceros, Osmanthus armatus, Passiflora sexflora, Persicaria decipiens, Persicaria hydropiper, Persicaria maculosa, Persicaria perfoliata, Persicaria salicifolia, Petunia integrifolia subsp. inflata, Phaseolus vulgaris, Phlogacanthus curviflorus, Photinia serratifolia, Physalis alkekengi, Picramnia antidesma, Picramnia hirsuta, Picramnia teapensis, Picramnia teapensis Tul., Picrasma quassioides, Pinalia stricta, Pisum sativum, Plantago coronopus, Plantago lanceolata, Pleione bulbocodioides, Pleione yunnanensis, Ploiarium alternifolium, Pluchea odora, Pneumatopteris pennigera, Polygoni cuspidati rhizoma et radix, Polygonum cuspidatum, Polygonum hypoleucum, Polygonum multiflorum, Polygonum perfoliatum, Pseudolarix amabilis, Psorospermum glaberrimum, Psorospermum laurentii, Psorospermum tenuifolium, Psychotria stachyoides, Quercus rubra, Radix Boehmeriae, Radix et Rhizoma Rhei, Radix et Rhizoma Rhei Praeparata, Ravenia spectabilis, Reynoutria japonica, Rhamnus cathartica, Rhamnus davurica, Rhamnus erythroxyloides, Rhamnus formosana, Rhamnus frangula [Syn. Frangula alnus], Rhamnus frangula, Rhamnus hirtella, Rhamnus kurdica, Rhamnus nakaharae, Rhamnus nepalensis, Rhamnus pallasii, Rhamnus prinoides, Rhamnus procumbens, Rhamnus punctata, Rhamnus saxatilis, Rhamnus thymifolius, Rhamnus triquetra, Rhamnus wightii, Rheum acuminatum, Rheum alexandrae, Rheum altaicum, Rheum australe, Rheum compactum, Rheum coreanum, Rheum delavayi, Rheum emodi [Syn. Rheum australe], Rheum franzenbachii, Rheum hotaoense, Rheum inopinatum, Rheum kialense, Rheum likiangense, Rheum nanum, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum spp., Rheum subacaule, Rheum sublanceolatum, Rheum tanguticum, Rheum tataricum, Rheum undulatum, Rheum uninerve, Rheum wittrockii, Rheum wittrocki, Rheum yunnanense, Rhizoma fagopyri cymose, Rhodiola viridula, Rhododendron dahuricum, Rosa pomifera, Rumex abyssinicus, Rumex acetosa, Rumex acetosella, Rumex aegyptiacus, Rumex alpinus, Rumex bequaertii, Rumex chalepensis, Rumex crispus, Rumex cristatus, Rumex cyprius, Rumex dentatus, Rumex japonicus, Rumex lanceolatus, Rumex limoniastrum, Rumex nepalensis, Rumex patientia, Rumex pictus, Rumex pulcher, Rumex ruwenzoriensis, Rumex scutatus, Rumex simpliciflorus, Rumex spp., Rumex tuberosus, Rumex usambarensis, Rumex vesicarius, Salvia aurea, Salvia greggii, Sargentodoxa cuneata, Sargentodoxae caulis, Saururus chinensis, Schistostephium artemisiifolium, Scilla socialis, Selaginella labordei, Semen pharbitidis, Senecio behnii, Senecio bonariensis, Senecio praecox, Senecio subdentatus, Senna alata, Senna alexandrina, Senna angustisiliqua, Senna artemisioides, Senna auriculata, Senna corymbosa, Senna didymobotrya, Sennae folium, Senna lindheimeriana, Senna macranthera, Senna obtusifolia, Senna occidentalis, Senna petersiana, Senna podocarpa, Senna septemtrionalis, Senna siamea, Senna sophera, Senna spectabilis, Senna tora, Simaba orinocensis, Smythea bombaiensis, Solanum cleistogamum, Sophorae tonkinensis radix et rhizome, Spatholobus suberectus, Sphaerella rosigena, Spinacia oleracea, Stevia berlandieri, Stevia ovata, Syzygium australe, Tagetes filifolia, Tanacetum coccineum, Taraxacum kok-saghyz, Taraxacum platycarpum subsp. hondoense, Tetradium ruticarpum, Teucrium yemense, Thymelaea hirsute, Tribuli fructus, Tribulus terrestris, Uvaria afzelii, Vatairea heteropteran, Ventilago calyculata, Ventilago denticulata, Ventilago leiocarpa, Ventilago madraspatana, Verbascum thapsiforme, Vismia guianensis, Vismia guineensis, Vismia laurentii, Vismia orientalis, Vitis vinifera, Wilbrandia ebracteata, Xanthii fructus, Xerochrysum viscosum, Angelica archangelica, Frangula purshiana, Geranium maculatum, Rumex crispus, Senecio congestus, Senna alexandrina, Aloe africana, Aloe arborescens, Aloe castellorum, Aloe elgonica, Aloe ferox, Aloe rubroviolacea, Aloe spicata, Aloe succotrina, Asphodeline tenuior, Asphodelus aestivus, Asphodelus albus, Asphodelus fistulosus, Asphodelus ramosus, Asphodelus tenuifolius, Berchemia floribunda, Bergenia hissarica, Bulbine annua, Bulbine asphodeloides, Callisia fragrans, Cassia aldabrensis, Cassia grandis, Cassia javanica, Chamaecrista mimosoides, Commiphora socotrana, Frangula almus, Frangula purshiana, Kniphofia ensifolia, Kniphofia foliosa, Kniphofia insignis, Kniphofia isoetifolia, Kniphofia schimperi, Picramnia antidesma, Picramnia hirsute, Psidium guajava, Reynoutria japonica, Rhamnus alaternus, Rhamnus cathartica, Rhamnus davurica, Rheum acuminatum, Rheum alexandrae, Rheum altaicum, Rheum australe, Rheum compactum, Rheum coreanum, Rheum delavayi, Rheum franzenbachii, Rheum hotaoense, Rheum inopinatum, Rheum kialense, Rheum likiangense, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum subacaule, Rheum tanguticum, Rheum tataricum, Rheum undulatum, Rheum uninerve, Rheum wittrockii, Rheum yunnanense, Rumex cristatus, Rumex paulsenianus, Rumex scutatus, Selaginella delicatula, Senna alata, Senna alexandrina, Senna didymobotrya, Senna obtusifolia, Senna occidentalis, Senna petersiana, Senna reticulata, Senna siamea, Senna tora, Spatholobus suberectus, Stephania dinklagei, Xanthorrhoea australis, Cichorium intybus, Cassia occidentalis, Morinda royoc, Galium aparine, Bryophyllum pinnatum, Anthema tinctoria, Aegle marmelos, Cymbopogon spp, Wedelia trilobata, Angelica archangelica, Vitis tilifolia, Mirabilis jalapa, Centipeda minima, Bauhinia manca, Juniperus drupacea, Forsythia suspensa, Citrus sinensis, Hylocomium splendens, Carthamus tinctorius, Rosmarinus officinalis, Oryza sativa, Trifolium pratense cv. Redman, Hordeum vulgare cv. Seco, Nepenthes x hookeriana, Musa acuminata, Pseudotsuga menziesii var. menziesii, Theobroma cacao, Hordeum vulgare cv. Seco, Nepenthes x hookeriana, Musa acuminata, Pseudotsuga menziesii var. menziesii, Penstemon centranthifolius, Pisum sativum, Glycine max, Avena sativa, Malus domestica, Gardenia jasminoides, Aloe ferox, Oryza sativa, Coffea arabica, Carthamus tinctorius, Macrotyloma uniflorum, Solanum melongena, Citrus unshiu, Hypericum perforatum, Curcuma longa, Nypa fruticans, Secale cereale cv. Aroostook, Persicaria tinctoria, Cannabis sativa, Solanum lycopersicum, Citrus clementina, Trifolium pratense cv. Redman, Uncaria tomentosa, Auricularia auricula-judae, Citrus sinensis, Cycas revoluta, Momordica charantia, Brassica carinata, Gaillardia pulchella, Nelumbo nucifera, Raphanus sativus var. niger, Lepidium meyenii, Zea mays, Nicotiana tabacum, Agaricus subrufescens, Linum usitatissimum, Paeonia lactiflora, Capsicum annuum, Larrea tridentata, Mirabilis jalapa, Catharanthus roseu, Medicago sativa subsp. sativa, Solanum americanum, Coffea arabica, Momordica charantia, Silybum marianum, Theobroma cacao, Aloe ferox, Gardenia jasminoides, Anacardium occidentale, Capsicum annuum, Paeonia lactiflora, Citrus clementina, Hordeum vulgare cv. Seco, Erucastrum gallicum, Linum usitatissimum, Asclepias syriaca, Avena sativa, Hypericum perforatum, Curcuma longa, Zea mays, Larrea tridentata, Nicotiana tabacum, Eucalyptus globulus subsp. bicostata, Musa acuminata, Solanum melongena, Phyllanthus emblica, Solanum lycopersicum, Veronica polita, Alcea rosea, Plukenetia volubilis, Euphorbia tirucalli, Neolamarckia cadamba, Citrus limon, Catharanthus roseus, Mirabilis jalapa, Podocarpus spicatus, Tripterygium wilfordii, Spatholobus suberectus, Crinum moorei, Juniperus monosperma, Pleurotus ostreatus, Gypsophila perfoliate, Clausena excavate, Gynura procumbens, Trifolium pratense cv. Redman, Oryza sativa, Rosmarinus officinalis, Nelumbo nucifera, Carthamus tinctorius, Citrus unshiu, Hylocomium splendens, Citrus sinensis, Juniperus drupacea, Forsythia suspensa, Momordica charantia, Silybum marianum, Aloe ferox, Citrus clementina, Hordeum vulgare cv. Seco, Erucastrum gallicum, Linum usitatissimum, Asclepias syriaca, Hypericum perforatum, Larrea tridentata, Eucalyptus globulus subsp. bicostata, Phyllanthus emblica, Veronica polita, Alcea rosea, Neolamarckia cadamba, Catharanthus roseus, Podocarpus spicatus, Tripterygium wilfordii, Juniperus monosperma, Pleurotus ostreatus, Gypsophila perfoliate, Clausena excavate, Gynura procumbens, Trifolium pratense cv. Redman, Rosmarinus officinalis, Nelumbo nucifera, Citrus unshiu, Hylocomium splendens, Citrus sinensis, Juniperus drupacea, Forsythia suspensa, or a combination thereof. In some embodiments, the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof. In some embodiments, the oral composition is formulated as a dietary supplement, food ingredient or additive, a medical food, nutraceutical or pharmaceutical composition. In some embodiments, the oral composition is in a dosage form configured as one of a soft-gel capsule, a hard capsule, liquid, powder, and a tablet. In some embodiments, the oral composition comprises 10% to 99% w/w of the plant extract. In some embodiments, the plant extract further comprises a preservative. In some embodiments, the plant extract further comprises a sweetener. In some embodiments, the plant extract further comprises a coloring agent. In some embodiments, the plant extract further comprises an additive. In some embodiments, the plant extract further comprises a flavoring agent.

Some embodiments relate to a method of treating, preventing, reducing, modulating, or ameliorating stress, symptoms of stress, or deleterious effects of chronic stress in a subject, in need thereof. In some embodiments, the method includes administering a therapeutically effect amount of a composition comprising the oral composition as described herein. In some embodiments, the plant extract of the oral composition is capable of activating a group II metabotropic receptor. In some embodiments, the plant extract activates an mGlu2 receptor. In some embodiments, the plant extract activates an mGlu3 receptor.

### BRIEF DESCRIPTION OF THE DRAWINGS

FIG. 1 illustrates a graph depicting plant fractions with confirmed antagonism/inhibition activity.

FIG. 2 illustrates a graph depicting plant fractions with confirmed agonism/activation activity.

FIG. 3 illustrates a follow chart depicting a standardized bioassay protocol.

FIG. 4 illustrates a bar graph demonstrating an mGluR2 antagonist/NAM assay activity.

FIG. 5 illustrates a bar graph demonstrating an mGluR2 antagonist/PAM assay activity.

### DETAILED DESCRIPTION OF THE DISCLOSURE

This disclosure provides, among other things, compounds, compositions, including oral compositions, and methods for administering the compounds and compositions to modulate or treat stress or stress related symptoms or disorders. In some embodiments, the oral composition includes a therapeutically effectively amount of a compound or a plant extract and one or more carriers or excipients. In some embodiments, the method of treating, preventing, reducing, modulating, or ameliorating stress, symptoms of stress, or deleterious effects of chronic stress in a subject includes administering a composition as described herein. This disclosure also provides, among other things, oral compositions and methods for orally administering the compositions to modulate one or more group II metabotropic glutamate receptor. In some embodiments, the compound, plant extract, or composition as described herein is a positive allosteric modulator (PAM) of the group II metabotropic glutamate receptors. In some embodiments, the compound, plant extract, or composition as described herein is an antagonist of one or more group II metabotropic glutamate receptor. In some embodiments, the compound, plant extract, or composition as described herein is an agonist of one or more group II metabotropic glutamate receptor.

### Compositions

In some aspects, the disclosure provided herein provides for a composition comprising a plant extract and a carrier. In some embodiments, the composition is an oral composition. As used herein, an “extract” refers a composition containing a desired compound of interest which is separated from other substances present in the natural source material from which the oral composition was obtained. Plant extracts can be obtained from any plant tissue including a whole plant; plant part such as shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary); plant tissue (for example, vascular tissue, ground tissue, and the like); or cell (for example, guard cells, egg cells, and the like), and progeny and cultures or cell lines of the same. In some embodiments, the extract is generally recognized as safe for human consumption. Accordingly, in certain embodiments the extract is from an edible source. In this respect, the extract is an edible extract. Extracts can be prepared by freezing, grinding, macerating, pulverizing and/or fermenting the source material of interest, subjecting the source material to solvent extraction, and separating the insoluble material from soluble material. In this respect, an “extract” of the disclosure can be crude, fractionated, sub-fractionated, separated, isolated, enriched or purified, without being limited thereto.

In some embodiments, the plant extract may be a crude extract. As used herein, “crude” refers to compounds or molecules that have not been entirely separated from the components of the original composition in which it was present. In embodiments pertaining to fractions or sub-fractions, a molecule in crude extract may be subjected to partial separation to provide a less crude extract containing other substances. By comparison, the term “isolated” means that a compound or molecule is substantially enriched or purified with respect to the complex cellular milieu in which it naturally occurs, such as in a crude extract. When an isolated molecule is enriched or purified, the absolute level of purity is not critical and those skilled in the art can readily determine appropriate levels of purity according to the use to which the material is to be put. In some circumstances, the isolated molecule forms part of a composition (for example a more or less crude extract containing many other substances), which may for example contain other components. In other circumstances, the isolated molecule may be purified to essential homogeneity, for example as determined spectrophotometrically, by NMR or by chromatography (for example LC-MS).

Suitable solvents for preparing an extract include, e.g., n-pentane, hexane, butane, chloroform, dichloromethane, di-ethyl ether, acetonitrile, water, butanol, isopropanol, ethanol, methanol, glacial acetic acid, acetone, norflurane (HFA134a), ethyl acetate, dimethyl sulfoxide, heptafluoropropane (HFA227), and subcritical or supercritical fluids such as liquid carbon dioxide and water, or a combination thereof in any proportion. When solvents such as those listed above are used, the resultant extract typically contains non-specific lipid-soluble material. This can be removed by a variety of processes including “winterization”, which involves chilling to a specified temperature, typically −20° C. followed by filtration or centrifugation to remove waxy ballast, extraction with subcritical or supercritical carbon dioxide or non-polar solvents (e.g., hexane) and by distillation.

In some embodiments, the plant extract may be selected from Rheum officinale, Phyllanthus emblica, Malus domestica, Tripterygium wilfordii, Aloe barbadensis, Theobroma cacao, Chaenomeles sinensis, Hypericum perforatum, Citrus unshiu, Nelumbo nucifera, Acacia luederitzii, Cassia abbreviate, Cassia fistula, Colophospermum mopane, Julbernardia globiflora, Habranthus brachyandrus, Monactis macbridei, Arnica acaulis, Arnica montana, Daucus carota, Bauhinia manca, Botrytis cinerea, Brosimum acutifolium, Broussonetia kazinoki, Broussonetia papyrifera, Dalbergia latifolia, Dracaena cambodiana, Dracaena draco, Narcissus pseudonarcissus, Phanera guianensis, Rohdea chinensis, Soymida febrifuga, Taxus brevifolia, Tupistra chinensis, Abri herba, Abrus pulchellus subsp. Cantoniensis, Achyranthes bidentata, Aconitum seravschanicum, Aconitum tasiromontanum, Acori tataninowii rhizome, Acorus gramineus, Acorus tatarinowii, Actinidia chinensis planch, Adansonia grandidieri, Aframomum giganteum, Aizopsis selkiana, Albizia myriophylla, Alisma plantago-aquatica subsp. orientale, Aloe castellorum, Alpiniae officinarum rhizome, Amaranthus hypochondriacus, Ampelopsis japonica, Amphorogyne spicata, Anaphalis lacteal, Anchusa azurea, Andrographis laxiflora, Andrographis neesiana, Anemarrhena asphodeloides, Aphyllocladus denticulatus, Aralia decaisneana, Artemisia annua, Artemisia parviflora, Artemisia scoparia [Syn. Artemisia capillaris v, Artemisia scoparia, Aspidosperma discolor, Asteris radix et rhizome, Aster sedifolius, Aster tataricus, Atadinus libanoticus, Baccharis potosina, Balanophora laxiflora, Bassia muricata, Benincasa hispida, Beta vulgaris vulgaris, Bistorta elliptica, Bistorta officinalis, Boehmeriae rhizoma et radix, Boehmeria nivea, Boehmeria platanifolia [Syn. Boehmeria tricuspis, Boehmeria platanifolia, Bolbostemae paniculati bulbus, Bolbostemma paniculatum, Brassica rapa, Bridelia stipularis, Canarium bengalense, Carduus tenuiflorus, Cascara sagrada, Cassia alata L., Cassia angolensis, Cassiae semen, Cassia fastuosa, Cassia javanica, Cassia obtusifolia, Cassia occidentalis, Cassia speciosa, Cassia tora L., Cassia tora, Cassia torosa L., Catalpa longissima, Caulerpa peltata, Caulis Polygoni Multiflori, Caulis Sargentodoxae, Caulocystis cephalornithos, Cephaelis ipecacuanha, Cephalotaxus griffithii, Chaiturus marrubiastrum, Chamaecrista mimosoides, Chamaecrista nigricans, Chamaecrista nomame, Chamaecrista pumila, Cheilanthes argentea, Chenopodium quinoa, Cichorium intybus, Cirsium oligophyllum, Clibadium sprucei, Coreopsis tinctoria, Corydalis rhizoma, Cranfillia fluviatilis, Cratoxylum formosum, Cremastra appendiculata, Cynanchum viminale, Dalbergia hupeana, Dalbergia lanceolaria subsp. paniculata, Degenia velebitica, Delphinium nuttallianum, Delphinium virgatum, Dendriopoterium menendezii, Dendrobium thyrsiflorum, Derris canarensis, Derris cuneifolia, Derris oblonga, Dianthi herba, Dianthus caryophyllus, Dictyopteris australis, Dillenia indica, Dioscoreae bulbiferae, Dipsacus azureus, Dodonaea viscosa, Dysoxylum cumingianum, Dysoxylum muelleri, Elymus repens, Enceliopsis argophylla, Endophytic aspergillus, Epimedium koreanum, Epimrdii herba, Eria mollis, Eriocaulon buergerianum, Eriocaulon sieboldianum, Erythrina lysistemon, Erythrophleum ivorense, Euodia bodinieri, Euodia officinalis, Euodia ruticarpa, Fagopyrum esculentum, Fagopyrum tataricum, Fallopia multiflora, Fallopia sachalinensis, Flacourtia jangomas, Frangula alnus, Frangula purshiana, Fridericia triplinervia, Garcinia polyantha, Gleichenia quadripartita, Glycyrrhiza uralensis, Gnaphalii affinis herba, Grangea maderaspatana, Hedera helix, Helichrysum chionosphaerum, Helichrysum plicatum, Heynea trijuga, Hoveniae dulcis semen, Hymenoxys lemmonii, Hypericum prolificum, Hypericum sampsonii, Isatdis radix, Isatis tinctoria, Jacquemontia paniculate, Juniperus formosana, Justicia secunda, Lactuca plumieri, Lactuca sativa, Lanaria lanata, Larix kaempferior, Lavandula vera, Leptocereus quadricostatus, Lespedeza tomentosa, Ligularia pleurocaulis, Lilii bulbus, Lilium brownii f. e. brown var. colchesteri wils, Lilium brownii var. viridulum [Syn. Lilium brown, Lilium brownii var. viridulum, Lobelia berlandieri, Loranthus pentandrus, Lupinus corymbosus, Lycii cortex, Maclura tinctoria, Magnolia figo, Magnolia ovata, Mandragora autumnalis, Melicope triphylla, Mentha arvensis, Mentha canadensis, Menthae herba, Metrodorea nigra, Miliusa velutina, Mitracarpus scaber, Muehlenbeckia tamnifolia, Muntingia calabura, Myrsine africana, Nekemias grossedentata, Neobalanocarpus heimii, Nepeta granatensis, Nepeta parnassica, Nuxia floribunda, Onobrychis arenaria, Oryctes monoceros, Osmanthus armatus, Passiflora sexflora, Persicaria decipiens, Persicaria hydropiper, Persicaria maculosa, Persicaria perfoliata, Persicaria salicifolia, Petunia integrifolia subsp. inflata, Phaseolus vulgaris, Phlogacanthus curviflorus, Photinia serratifolia, Physalis alkekengi, Picramnia antidesma, Picramnia hirsuta, Picramnia teapensis, Picramnia teapensis Tul., Picrasma quassioides, Pinalia stricta, Pisum sativum, Plantago coronopus, Plantago lanceolata, Pleione bulbocodioides, Pleione yunnanensis, Ploiarium alternifolium, Pluchea odora, Pneumatopteris pennigera, Polygoni cuspidati rhizoma et radix, Polygonum cuspidatum, Polygonum hypoleucum, Polygonum multiflorum, Polygonum perfoliatum, Pseudolarix amabilis, Psorospermum glaberrimum, Psorospermum laurentii, Psorospermum tenuifolium, Psychotria stachyoides, Quercus rubra, Radix Boehmeriae, Radix et Rhizoma Rhei, Radix et Rhizoma Rhei Praeparata, Ravenia spectabilis, Reynoutria japonica, Rhamnus cathartica, Rhamnus davurica, Rhamnus erythroxyloides, Rhamnus formosana, Rhamnus frangula [Syn. Frangula alnus], Rhamnus frangula, Rhamnus hirtella, Rhamnus kurdica, Rhamnus nakaharae, Rhamnus nepalensis, Rhamnus pallasii, Rhamnus prinoides, Rhamnus procumbens, Rhamnus punctata, Rhamnus saxatilis, Rhamnus thymifolius, Rhamnus triquetra, Rhamnus wightii, Rheum acuminatum, Rheum alexandrae, Rheum altaicum, Rheum australe, Rheum compactum, Rheum coreanum, Rheum delavayi, Rheum emodi [Syn. Rheum australe], Rheum franzenbachii, Rheum hotaoense, Rheum inopinatum, Rheum kialense, Rheum likiangense, Rheum nanum, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum spp., Rheum subacaule, Rheum sublanceolatum, Rheum tanguticum, Rheum tataricum, Rheum undulatum, Rheum uninerve, Rheum wittrockii, Rheum wittrocki, Rheum yunnanense, Rhizoma fagopyri cymose, Rhodiola viridula, Rhododendron dahuricum, Rosa pomifera, Rumex abyssinicus, Rumex acetosa, Rumex acetosella, Rumex aegyptiacus, Rumex alpinus, Rumex bequaertii, Rumex chalepensis, Rumex crispus, Rumex cristatus, Rumex cyprius, Rumex dentatus, Rumex japonicus, Rumex lanceolatus, Rumex limoniastrum, Rumex nepalensis, Rumex patientia, Rumex pictus, Rumex pulcher, Rumex ruwenzoriensis, Rumex scutatus, Rumex simpliciflorus, Rumex spp., Rumex tuberosus, Rumex usambarensis, Rumex vesicarius, Salvia aurea, Salvia greggii, Sargentodoxa cuneata, Sargentodoxae caulis, Saururus chinensis, Schistostephium artemisiifolium, Scilla socialis, Selaginella labordei, Semen pharbitidis, Senecio behnii, Senecio bonariensis, Senecio praecox, Senecio subdentatus, Senna alata, Senna alexandrina, Senna angustisiliqua, Senna artemisioides, Senna auriculata, Senna corymbosa, Senna didymobotrya, Sennae folium, Senna lindheimeriana, Senna macranthera, Senna obtusifolia, Senna occidentalis, Senna petersiana, Senna podocarpa, Senna septemtrionalis, Senna siamea, Senna sophera, Senna spectabilis, Senna tora, Simaba orinocensis, Smythea bombaiensis, Solanum cleistogamum, Sophorae tonkinensis radix et rhizome, Spatholobus suberectus, Sphaerella rosigena, Spinacia oleracea, Stevia berlandieri, Stevia ovata, Syzygium australe, Tagetes filifolia, Tanacetum coccineum, Taraxacum kok-saghyz, Taraxacum platycarpum subsp. hondoense, Tetradium ruticarpum, Teucrium yemense, Thymelaea hirsute, Tribuli fructus, Tribulus terrestris, Uvaria afzelii, Vatairea heteropteran, Ventilago calyculata, Ventilago denticulata, Ventilago leiocarpa, Ventilago madraspatana, Verbascum thapsiforme, Vismia guianensis, Vismia guineensis, Vismia laurentii, Vismia orientalis, Vitis vinifera, Wilbrandia ebracteata, Xanthii fructus, Xerochrysum viscosum, Angelica archangelica, Frangula purshiana, Geranium maculatum, Rumex crispus, Senecio congestus, Senna alexandrina, Aloe africana, Aloe arborescens, Aloe castellorum, Aloe elgonica, Aloe ferox, Aloe rubroviolacea, Aloe spicata, Aloe succotrina, Asphodeline tenuior, Asphodelus aestivus, Asphodelus albus, Asphodelus fistulosus, Asphodelus ramosus, Asphodelus tenuifolius, Berchemia floribunda, Bergenia hissarica, Bulbine annua, Bulbine asphodeloides, Callisia fragrans, Cassia aldabrensis, Cassia grandis, Cassia javanica, Chamaecrista mimosoides, Commiphora socotrana, Frangula alnus, Frangula purshiana, Kniphofia ensifolia, Kniphofia foliosa, Kniphofia insignis, Kniphofia isoetifolia, Kniphofia schimperi, Picramnia antidesma, Picramnia hirsute, Psidium guajava, Reynoutria japonica, Rhamnus alaternus, Rhamnus cathartica, Rhamnus davurica, Rheum acuminatum, Rheum alexandrae, Rheum altaicum, Rheum australe, Rheum compactum, Rheum coreanum, Rheum delavayi, Rheum franzenbachii, Rheum hotaoense, Rheum inopinatum, Rheum kialense, Rheum likiangense, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum subacaule, Rheum tanguticum, Rheum tataricum, Rheum undulatum, Rheum uninerve, Rheum wittrockii, Rheum yunnanense, Rumex cristatus, Rumex paulsenianus, Rumex scutatus, Selaginella delicatula, Senna alata, Senna alexandrina, Senna didymobotrya, Senna obtusifolia, Senna occidentalis, Senna petersiana, Senna reticulata, Senna siamea, Senna tora, Spatholobus suberectus, Stephania dinklagei, Xanthorrhoea australis, Cichorium intybus, Cassia occidentalis, Morinda royoc, Galium aparine, Bryophyllum pinnatum, Anthema tinctoria, Aegle marmelos, Cymbopogon spp, Wedelia trilobata, Angelica archangelica, Vitis tilifolia, Mirabilis jalapa, Centipeda minima, Bauhinia manca, Juniperus drupacea, Forsythia suspensa, Citrus sinensis, Hylocomium splendens, Carthamus tinctorius, Rosmarinus officinalis, Oryza sativa, Trifolium pratense cv. Redman, Hordeum vulgare cv. Seco, Nepenthes x hookeriana, Musa acuminata, Pseudotsuga menziesii var. menziesii, Theobroma cacao, Hordeum vulgare cv. Seco, Nepenthes x hookeriana, Musa acuminata, Pseudotsuga menziesii var. menziesii, Penstemon centranthifolius, Pisum sativum, Glycine max, Avena sativa, Malus domestica, Gardenia jasminoides, Aloe ferox, Oryza sativa, Coffea arabica, Carthamus tinctorius, Macrotyloma uniflorum, Solanum melongena, Citrus unshiu, Hypericum perforatum, Curcuma longa, Nypa fruticans, Secale cereale cv. Aroostook, Persicaria tinctoria, Cannabis sativa, Solanum lycopersicum, Citrus clementina, Trifolium pratense cv. Redman, Uncaria tomentosa, Auricularia auricula-judae, Citrus sinensis, Cycas revoluta, Momordica charantia, Brassica carinata, Gaillardia pulchella, Nelumbo nucifera, Raphanus sativus var. niger, Lepidium meyenii, Zea mays, Nicotiana tabacum, Agaricus subrufescens, Linum usitatissimum, Paeonia lactiflora, Capsicum annuum, Larrea tridentata, Mirabilis jalapa, Catharanthus roseu, Medicago sativa subsp. sativa, Solanum americanum, Coffea arabica, Momordica charantia, Silybum marianum, Theobroma cacao, Aloe ferox, Gardenia jasminoides, Anacardium occidentale, Capsicum annuum, Paeonia lactiflora, Citrus clementina, Hordeum vulgare cv. Seco, Erucastrum gallicum, Linum usitatissimum, Asclepias syriaca, Avena sativa, Hypericum perforatum, Curcuma longa, Zea mays, Larrea tridentata, Nicotiana tabacum, Eucalyptus globulus subsp. bicostata, Musa acuminata, Solanum melongena, Phyllanthus emblica, Solanum lycopersicum, Veronica polita, Alcea rosea, Plukenetia volubilis, Euphorbia tirucalli, Neolamarckia cadamba, Citrus limon, Catharanthus roseus, Mirabilis jalapa, Podocarpus spicatus, Tripterygium wilfordii, Spatholobus suberectus, Crinum moorei, Juniperus monosperma, Pleurotus ostreatus, Gypsophila perfoliate, Clausena excavate, Gynura procumbens, Trifolium pratense cv. Redman, Oryza sativa, Rosmarinus officinalis, Nelumbo nucifera, Carthamus tinctorius, Citrus unshiu, Hylocomium splendens, Citrus sinensis, Juniperus drupacea, Forsythia suspensa, Momordica charantia, Silybum marianum, Aloe ferox, Citrus clementina, Hordeum vulgare cv. Seco, Erucastrum gallicum, Linum usitatissimum, Asclepias syriaca, Hypericum perforatum, Larrea tridentata, Eucalyptus globulus subsp. bicostata, Phyllanthus emblica, Veronica polita, Alcea rosea, Neolamarckia cadamba, Catharanthus roseus, Podocarpus spicatus, Tripterygium wilfordii, Juniperus monosperma, Pleurotus ostreatus, Gypsophila perfoliate, Clausena excavate, Gynura procumbens, Trifolium pratense cv. Redman, Rosmarinus officinalis, Nelumbo nucifera, Citrus unshiu, Hylocomium splendens, Citrus sinensis, Juniperus drupacea, Forsythia suspensa, or a combination thereof. In some embodiments, the plant extract includes a purified or crude extract. In some embodiments, the extract may be derived directly from seeds. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Rheum officinale. In some embodiments, the plant extract includes a purified or crude extract of Rheum officinale. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Phyllanthus emblica. In some embodiments, the plant extract includes a purified or crude extract of Phyllanthus emblica. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Malus domestica. In some embodiments, the plant extract includes a purified or crude extract of Malus domestica. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Tripterygium wilfordii. In some embodiments, the plant extract includes a purified or crude extract of Tripterygium wilfordii. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Aloe barbadensis. In some embodiments, the plant extract includes a purified or crude extract of Aloe barbadensis. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Theobroma cacao. In some embodiments, the plant extract includes a purified or crude extract of Theobroma cacao. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Chaenomeles sinesis. In some embodiments, the plant extract includes a purified or crude extract of Chaenomeles sinesis. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Hypericum perforatum. In some embodiments, the plant extract includes a purified or crude extract of Hypericum perforatum. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Citrus unshiu. In some embodiments, the plant extract includes a purified or crude extract of Citrus unshiu In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Chaenomeles sinensis. In some embodiments, the plant extract includes a purified or crude extract of Chaenomeles sinensis. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Nelumbo nucifera. In some embodiments, the plant extract includes a purified or crude extract of Nelumbo nucifera. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Inula helenium. In some embodiments, the plant extract includes a purified or crude extract of Inula helenium. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Acacia luederitzii. In some embodiments, the plant extract includes a purified or crude extract of Acacia luederitzii. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Cassia abbreviate. In some embodiments, the plant extract includes a purified or crude extract of Cassia abbreviate. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Cassia fistula. In some embodiments, the plant extract includes a purified or crude extract of Cassia fistula. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Colophospermum mopane. In some embodiments, the plant extract includes a purified or crude extract of Colophospermum mopane. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Solidago Canadensis. In some embodiments, the plant extract includes a purified or crude extract of Solidago Canadensis. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Julbernardia globiflora. In some embodiments, the plant extract includes a purified or crude extract of Julbernardia globiflora. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Habranthus brachyandrus. In some embodiments, the plant extract includes a purified or crude extract of Habranthus brachyandrus. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Monactis macbridei. In some embodiments, the plant extract includes a purified or crude extract of Monactis macbridei. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Arnica acaulis. In some embodiments, the plant extract includes a purified or crude extract of Arnica acaulis. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the plant extract includes Arnica montana. In some embodiments, the plant extract includes a purified or crude extract of Arnica montana. In some embodiments, the extract may be derived directly from whole seeds. In some embodiments, the extract may be derived directly from leaves. In some embodiments, the extract may be derived directly from pulp. In some embodiments, the extract may be derived directly from tubers. In some embodiments, the extract may be derived directly from flowers. In some embodiments, the extract may be derived directly from roots. In some embodiments, the extract may be derived directly from roots. In some embodiments, the seeds, leaves, flowers, pulp, tubers and/or roots may be ground into a powder, which is then heated to a temperature selected from within a range of about 30° C. to about 100° C., to remove oils prior to proceeding with an extraction step.

In some embodiments, the oral composition includes an extract from one or more plant source. In some embodiments, the oral composition includes an extract from two or more plant sources. In some embodiments, the oral composition includes an extract from three or more plant sources.

In some embodiments, the extract as described herein includes juices, infusions, and fermentation residues. These products fermentation plant-derived extracts or processed fractions thereof find use in consumable compositions such as health-promoting compositions or tonics for human and animals. “Plant-derived” as used herein refers to sourcing a compound from a plant, and may or may not refer to a pure extract from the plant.

In some embodiments, a compound of the disclosure is represented by a compound of Formula (I), or a salt thereof:

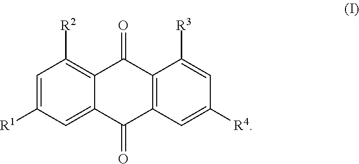

In some embodiments, R¹, R², R³, and R⁴ are each independently selected from hydrogen, deuterium, hydroxyl, halogen, keto, optionally substituted ester, optionally substituted aryl, optionally substituted alkoxy, optionally substituted C₁-C₆-alkyl, optionally substituted —O—C₁-C₆-alkyl, or optionally substituted C₃-C₆ cycloalkyl. In some embodiments, R¹, R², and R³ are each hydroxyl and R⁴ is optionally substituted C₁-C₆-alkyl. In some embodiments, R¹ is hydrogen, R² and R³ are hydroxyl and R⁴ is optionally substituted O—C₁-C₆-alkyl. In some embodiments, R¹ and R⁴ are hydrogen and R² and R³ are hydroxyl. In some embodiments, R¹ is hydrogen and R², R³, and R⁴ are keto. In some embodiments, R¹ is hydrogen, R² and R³ are hydroxyl, and R⁴ are keto. In some embodiments, R¹, R², R³ are hydroxyl, and R⁴ is optionally substituted C₁-C₆-alkyl.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyls. The alkyl group may be substituted or unsubstituted.

As used herein, “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene, and azulene. An aryl group may be substituted or unsubstituted.

The term “halogen” or “halo” as used herein, means any one of the radio-stable atoms of column 7 of the Periodic Table of the Elements, such as chloro (CI), fluoro (F), bromo (Br) and iodo (I) groups.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, mono-substituted amino group and di-substituted amino group, and protected derivatives thereof.

In some embodiments, the compound of Formula (I) are a compound capable of being useful as a modulator of a metabotropic glutamate receptor. In some embodiments, the modulator is capable of being an agonist of mGlu2. In some embodiments, the modulator is capable of being an agonist of mGlu3. In some embodiments, the modulator is capable of being an antagonist of mGlu2. In some embodiments, the modulator is capable of being an antagonist of mGlu3.

In some embodiments, a compound of Formula (I), a compound of the disclosure, and a plant extract includes a compound selected from Table A:

TABLE A

| Cmpd | |
|---|---|
| ID | Structure |
| 1 | 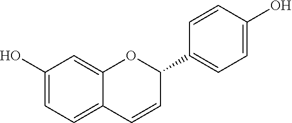 [SMILES: Oc1ccc([C@@H]2C=Cc3ccc(O)cc3O2)cc1] |
| 2 | 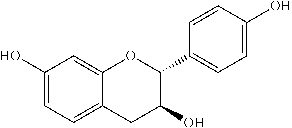 [SMILES: Oc1ccc([C@H]2Oc3cc(O)ccc3C[C@@H]2O)cc1] |
| 3 | 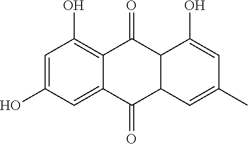 [SMILES: CC1=CC2C(=O)c3cc(O)cc(O)c3C(=O)C2C(O)=C1] |
| 4 | 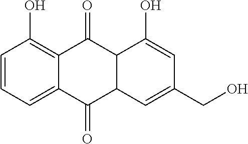 [SMILES: O=C1c2cccc(O)c2C(=O)C2C(O)=CC(CO)=CC12] |
| 5 | 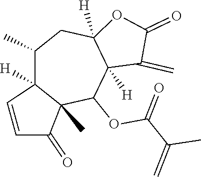 [SMILES: C=C(C)C(=O)OC1[C@@H]2C(=C)C(=O)O[C@@H]2C[C@@H](C)[C@@H]2C=CC(=O)[C@@]12C] |
| 6 | 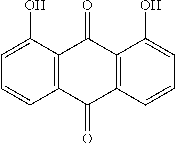 [SMILES: O=C1c2cccc(O)c2C(=O)c2c(O)cccc21] |
| 7 | 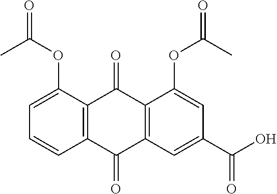 [SMILES: CC(=O)Oc1cccc2c1C(=O)c1c(OC(C)=O)cc(C(=O)O)cc1C2=O] |
| 8 | 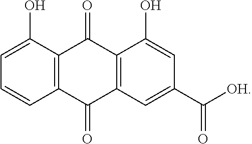 [SMILES: O=C(O)c1cc(O)c2c(c1)C(=O)c1cccc(O)c1C2=O] |
| 9 | 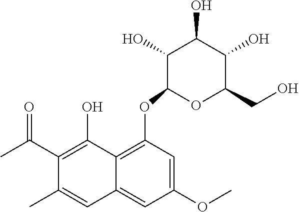 [SMILES: COc1cc(O[C@@H]2O[C@H](CO)[C@@H](O)[C@H](O)[C@H]2O)c2c(O)c(C(C)=O)c(C)cc2c1] |
| 10 | 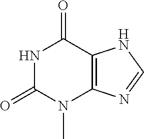 |
| 11 | 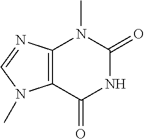 |
| 12 | 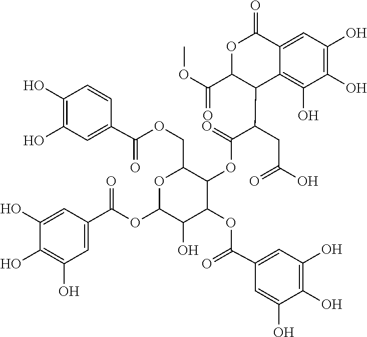 [SMILES: COC(=O)C1OC(=O)c2cc(O)c(O)c(O)c2C1C(CC(=O)O)C(=O)OC1C(COC(=O)c2ccc(O)c(O)c2)OC(OC(=O)c2cc(O)c(O)c(O)c2)C(O)C1OC(=O)c1cc(O)c(O)c(O)c1] |
| 13 | 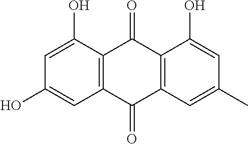 [SMILES: Cc1cc(O)c2c(c1)C(=O)c1cc(O)cc(O)c1C2=O] |

In some aspects, the disclosure provided herein provides for a composition comprising a compound of Table A and a carrier. The term “carrier” as used herein means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) lipids and/or phospholipids; and (23) other nontoxic compatible substances employed in conventional formulations.

In some embodiments, the composition includes a compound of Table A that is a substantially pure compound. In some embodiments, the composition includes less than about 5%, 4.5%, 4.0%, 3.5%, 3.0%, 2.5%, 2.0%, 1.5%, 1.0%, 0.5%, 0.25%, 0.1% impurities, or ranges including and/or spanning the aforementioned values. As used herein, “substantially pure” refers to a state where one or more of the desired active compound or compounds have been isolated and/or concentrated to a high degree, with minimal undesired impurities present. In some embodiments, the compound is plant derived and may be concentrated in the plant source without necessarily extracting the compound entirely or in part from the plant. By way of a non-limiting example a plant containing compound from Table A may be processed to isolate a particular part of the plant that is rich in compound from Table A from different parts of the plant that is comparatively low in concentration of a compound of Table A. By way of another non-limiting example, a plant containing a compound of Table A may be heated or crushed whereupon any compound from Table A in the interior of the plant may be more accessible to chemical extraction or digestion.

In some embodiments, the composition includes a compound of Table A from about 0.1%, 0.25%, 0.50%, 0.75%, 1.0%, 1.5%, 2.5%, 5.0%, 7.5%, 10%, 12.5%, 15%, 17.5%, 20%, 22.5%, 25%, 27.5%, 30%, 32.5%, 35%, 37.5%, 40%, 45%, 50%, 60%, 70%, 80%, 90%, 95%, 99%, or ranges including and/or spanning the aforementioned values. In some embodiments, the composition includes a compound of Table A from about 10% to about 90%. In some embodiments, the composition includes a compound of Table A from about 20% to about 75%. In some embodiments, the composition includes a compound of Table A from about 25% to about 50%. In some embodiments, the composition includes a compound of Table A from about 15%. In some embodiments, the composition includes a compound of Table A from about 25%. In some embodiments, the composition includes a compound of Table A from about 50%. In some embodiments, the composition includes a compound of Table A from about 75%. In some embodiments, the composition includes a compound of Table A from about 90%.

In some embodiments, the composition is formulated as an oral composition. In some embodiments, the composition comprises one or more compounds of Table A and a carrier. In some embodiments, the composition comprises compound 1 and a carrier. In some embodiments, the composition comprises compound 2 and a carrier. In some embodiments, the composition comprises compound 3 and a carrier. In some embodiments, the composition comprises compound 4 and a carrier. In some embodiments, the composition comprises compound 5 and a carrier. In some embodiments, compound 1 is an antagonist of mGlu2. In some embodiments, compound 1 is an antagonist of mGlu3. In some embodiments, compound 2 is an antagonist of mGlu2. In some embodiments, compound 2 is an antagonist of mGlu3. In some embodiments, compound 3 is an antagonist of mGlu2. In some embodiments, compound 3 is an antagonist of mGlu3. In some embodiments, compound 4 is an antagonist of mGlu2. In some embodiments, compound 4 is an antagonist of mGlu3. In some embodiments, compound 5 is an antagonist of mGlu2. In some embodiments, compound 5 is an antagonist of mGlu3. In some embodiments, compound 6 is an antagonist of mGlu2. In some embodiments, compound 6 is an antagonist of mGlu3. In some embodiments, compound 7 is an antagonist of mGlu2. In some embodiments, compound 7 is an antagonist of mGlu3. In some embodiments, compound 8 is an antagonist of mGlu2. In some embodiments, compound 8 is an antagonist of mGlu3. In some embodiments, compound 9 is an antagonist of mGlu2. In some embodiments, compound 9 is an antagonist of mGlu3. In some embodiments, compound 10 is an antagonist of mGlu2. In some embodiments, compound 10 is an antagonist of mGlu3. In some embodiments, compound 11 is an antagonist of mGlu2. In some embodiments, compound 11 is an antagonist of mGlu3. In some embodiments, compound 12 is an antagonist of mGlu2. In some embodiments, compound 12 is an antagonist of mGlu3. In some embodiments, compound 11 is an antagonist of mGlu3. In some embodiments, compound 13 is an antagonist of mGlu2. In some embodiments, compound 13 is an antagonist of mGlu3. In some embodiments, compound 1 is an antagonist/negative allosteric modulator. In some embodiments, compound 2 is an antagonist/negative allosteric modulator. In some embodiments, compound 3 is an antagonist/negative allosteric modulator. In some embodiments, compound 4 is an antagonist/negative allosteric modulator. In some embodiments, compound 5 is an antagonist/negative allosteric modulator. In some embodiments, compound 6 is an antagonist/negative allosteric modulator. In some embodiments, compound 7 is an antagonist/negative allosteric modulator. In some embodiments, compound 8 is an antagonist/negative allosteric modulator. In some embodiments, compound 9 is an antagonist/negative allosteric modulator. In some embodiments, compound 10 is an antagonist/negative allosteric modulator. In some embodiments, compound 11 is an antagonist/negative allosteric modulator. In some embodiments, compound 12 is an agonist/positive allosteric modulator. In some embodiments, compound 13 is an agonist/positive allosteric modulator.

In some embodiments, the oral composition includes a salt form of a compound as described herein. A salt of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like; or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic camphorsulfonic acid, acid, 4-toluenesulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

In some embodiments, the oral composition includes a pharmaceutically acceptable salt form a compound as described herein. The term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic acid, acetic acid (AcOH), propionic acid, glycolic acid, pyruvic acid, malonic acid, maleic acid, fumaric acid, trifluoroacetic acid (TFA), benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluenesulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Pharmaceutical salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition, it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns. Likewise, it is understood that, in any compound described, all tautomeric forms are also intended to be included.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the oral compositions, formulations, and methods described herein include the use of crystalline forms, amorphous phases, and/or pharmaceutically acceptable salts, solvates, hydrates, and conformers of compounds of some embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. In specific embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein. Other forms in which the compounds of some embodiments can be provided include amorphous forms, milled forms and nano-particulate forms.

Likewise, it is understood that the compounds described herein, such as compounds of some embodiments, include the compound in any of the forms described herein (e.g., pharmaceutically acceptable salts, prodrugs, crystalline forms, amorphous form, solvated forms, enantiomeric forms, tautomeric forms, and the like).

In some embodiments, the composition may further include a preservative. In some embodiments, the preservative may be in an amount from about 0.01%, 0.05%, 0.1%, 0.15%, 0.20%, 0.25%, 0.50%, 0.75%, 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0% w/w, or ranges including and/or spanning the aforementioned values. In some embodiments, the preservative may be, but not limited to, the group selected from sodium benzoate, methyl parabens, propyl parabens, sodium nitrite, sulfur dioxide, sodium sorbate and potassium sorbate. Other suitable preservatives include, but are not limited to, salts of edetate, (also known as salts of ethylenediaminetetraacetic acid, or EDTA, such a disodium EDTA).

In some embodiments, the composition may further include a sweetener. In some embodiments, the sweetener may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the sweetener may include a natural sweetener. In some embodiments, the sweetener may include a synthetic sweetener. Non-limiting examples of sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, and mixtures thereof.

In some embodiments, the composition may further include a flavoring agent. In some embodiments, the flavoring agent may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the flavoring agent may include natural or artificial flavors. In a particular embodiment, the flavoring agent comprises an essential oil, such as an oil derived from a plant or a fruit, peppermint oil, spearmint oil, other mint oils, clove oil, cinnamon oil, oil of wintergreen, bay, thyme, cedar leaf, nutmeg, allspice, sage, mace, and almonds. In another particular embodiment, the flavoring agent comprises a plant extract or a fruit essence such as apple, banana, watermelon, pear, peach, grape, strawberry, raspberry, cherry, plum, pineapple, apricot, and mixtures thereof. In still another particular embodiment, the flavoring agent comprises a citrus flavor, such as an extract, essence, or oil of lemon, lime, orange, tangerine, grapefruit, citron, or kumquat.

In some embodiments, the composition may further include a coloring agent. In some embodiments, the coloring agent may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values.

In some embodiments, the composition may further include at least one additive. In some embodiments, the additive may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the at least one additive may be an emulsifier. In some embodiments, the additive may include one or more vitamin. In some embodiments, the additive may include one or more minerals. In some embodiments, the additive may include a stabilizer.

### Formulations

A composition comprising a plant extract or a compound of this disclosure can be combined with a carrier and provided in any suitable form for consumption by or administration to a subject. In this respect, the plant extract or compound is added as an exogenous ingredient or additive to the consumable. Suitable consumable forms include, but are not limited to, a dietary supplement, food ingredient or additive, a medical food, nutraceutical or pharmaceutical composition. In some embodiments, the plant extract or compound is provided in either a liquid or powder form.

A “food ingredient” or “food additive” as referred to herein includes an edible substance intended to result, directly or indirectly, in becoming a component of or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound as described herein, or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The oral compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In some embodiments, an oral composition as described herein may be administered at a dose in the range of about 0.1-200 mg/kg body weight. In some embodiments, an oral composition as described herein is administered at a dose in the range of about 0.1-1, 0.5-1, 0.1-10, 0.5-10, 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500, 60-600, 60-700, 60-800, 60-900, 60-1000, 70-80, 70-90, 70-100, 70-200, 70-300, 70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, an oral composition as described herein is administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 80, 90, or 95 mg/kg of the body weight. In some embodiments, an oral composition as described herein is administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, an oral composition as described herein is administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of a subjects body weight.

In some embodiments, an oral composition as described herein dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg-50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, an oral composition as described herein is administered at about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, an oral composition as described herein dose is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg, about 400 mg, about 500 mg, about 600 mg, about 700 mg, about 800 mg, about 900 mg, or about 1000 mg. In some embodiments, an oral composition as described herein dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

The liquid forms in which the compound or extract of the disclosure is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

When a compound or extract of the present disclosure is administered as pharmaceuticals, nutraceuticals, or dietary supplements to humans and animals, they can be given per se or as a composition containing, for example, 0.1 to 99% active ingredient in combination with an acceptable carrier. In some embodiments, the compound or extract of the present disclosure may be administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values.

A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound. In some embodiments, the food product includes one or more compounds as described herein and a fiber source. In some embodiments, the food product includes one or more compounds as described herein. In some embodiments, the food product is a food bar. In some embodiments, the food bar comprises from about 0.01% to about 20% (w/w) of one or more compounds as described herein. In some embodiments, the food bar comprises from about 0.1% to about 10% (w/w) of one or more compounds as described herein. In some embodiments, the food product is a crisp. In some embodiments, the food crisp comprises from about 0.01% to about 20% (w/w) of one or more compounds as described herein. In some embodiments, the food crisp comprises from about 0.1% to about 10% (w/w) of one or more compounds as described herein.

The compounds, compositions and extracts of the present disclosure can be prepared as nutritional formulations such as foods, including medical or functional foods. A “medical or functional food” is defined as being consumed as part of a usual diet but which has been demonstrated to have physiological benefits and/or to reduce the risk of a disease or condition such as a chronic disease, beyond basic nutritional functions. By way of example, but not limitation, medical foods and functional foods may include one or more of the following ingredients: a compound described herein, a composition described herein, an extract described herein, vitamins, minerals, herbs, botanicals, amino acids, dietary substances intended to supplement the diet by increasing total dietary intake, concentrates, metabolites, constituents, extracts or combinations of any of the foregoing. Dietary supplements may also be incorporated into food stuffs, such as functional foods designed to promote health or to prevent disease or disorders. If administered as a medicinal preparation, the composition can be administered, either as a prophylaxis or treatment, to a patient in any of a number of methods. In some embodiments, the compositions described herein may be administered alone or in combination with other additives or pharmaceutical agents and can be combined with a physiologically acceptable carrier thereof. The effective amount and method of administration and aim of the particular formulation can vary based on the individual subject, the stage of the disease or condition, and other factors evident to one skilled in the art. In the case of a pharmaceutical formulation as well as a nutraceutical formulation, during the course of the treatment, the concentration of the subject compositions may be monitored (for example, blood plasma levels may be monitored) to ensure that the desired level is maintained.

In some embodiments, food product includes a carbohydrate. A variety of carbohydrates are used in food products, such as various sugars and starches. Carbohydrates are an important source of energy for the body, including complex carbohydrates (like whole grains and vegetables) and simple carbohydrates (like sugar and refined grains). There are several common carbohydrates that are used in food products, including: starch, sugar, fructose, maltodextrin, dextrose, corn syrup, oligosaccharides, cellulose, complex carbohydrates, such as inulin that may be added as a prebiotic fiber in processed foods. According to the disclosure, the concentration of carbohydrates may vary depending on the intended use of a product. In some embodiments, carbohydrates may comprise from about 1% by weight to about 95% by weight of a food product. In some embodiments, carbohydrates may comprise from about 1% by weight to about 95% by weight of a food product. In some embodiments, a food product may comprise carbohydrates in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a sugar. Sugars, such as high fructose corn syrup, sucrose, glucose syrup, dextrose, and other sweeteners are often added to processed foods to make them more palatable. Additionally, sugar alcohols or maltodextrins, may be added to food products to reduce caloric content and/or to enhance nutritional profile of the product. In some embodiments, a food product may comprise one or a plurality of saccharides that are slowly or incompletely digested by humans, if not totally indigestible. These sugars can include isomaltose, panose and branched oligomers having a degree of polymerization of four or greater. Additional non-limiting examples of sugars include sucrose, HFCS, fructose, brown sugar (which can be either partially or fully refined), powdered sugar (also known as confectioner's sugar), high fructose corn syrup, honey, molasses, maple syrup, agave nectar, coconut sugar, date sugar, fruit juice concentrates, maltodextrin, dextrose, glucose syrup, maple syrup, molasses, and lactose. According to the disclosure, the concentration of sugars may vary depending on the intended use of a product. In some embodiments, sugar may comprise from about 1% by weight to about 95% by weight of a food product. In some embodiments, the sugar(s) component comprises from about 2% to about 10% by weight of the composition. In some embodiments, sugars may comprise from about 1% by weight to about 95% by weight of a food product. In some embodiments, a food product may comprise sugars in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product includes a fiber source. In some embodiments, the food product includes from about 5% to about 30% (w/w) fiber. In some embodiments, the food product includes at least 10% fiber. In some embodiments, the food product includes at least 15% fiber. In some embodiments, the food product includes at least 20% fiber. In some embodiments, the food product includes at least 30% fiber. In some embodiments, the fiber may be selected from psyllium husk, inulin, beta-glucans, acacia fiber, pectin, wheat dextrin, cellulose, fructooligosaccharides, guar gum, fruit fiber, or a combination thereof. In some embodiments, the fiber is a soluble dietary fiber.

In some embodiments, the food product may include a protein. Proteins may be included in a range of concentrations depending on the product. Protein sources can include soy protein, soy flour, soy protein isolate, whey protein isolate, casein, gelatin, legume protein isolates, soy protein concentrate, egg albumin or egg white, wheat protein concentrate, legume protein concentrates and mixtures thereof. In some embodiments, meat products like beef jerky, sausages, or meatballs, may include proteins as a primary ingredient. Proteins may be included at concentrations ranging from about 10% to about 25% of the total ingredients. In dairy products like cheese, yogurt, or milk, proteins like casein or whey may be the primary ingredient. Proteins may be included at concentrations ranging from about 3% to about 15% of the total ingredients. In plant-based products like tofu, tempeh, or seitan, proteins from soy, peas, legumes, or wheat may be primary ingredients. Proteins may be included at concentrations ranging from about 10% to about 25% of the total ingredients. In energy bars and protein bars, proteins like whey or soy, or any protein according to the disclosure may be added to provide a source of protein. Proteins may be included at concentrations ranging from about 10% to about 25% of the total ingredients. According to the disclosure, the concentration of proteins may vary depending on the intended use of a product. In some embodiments, proteins may comprise from about 1% by weight to about 95% by weight of a food product. In some embodiments, proteins may comprise from about 1% by weight to about 95% by weight of a food product. In some embodiments, a food product may comprise proteins in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a starch. In some embodiments, food products may additionally comprise a starch ingredient(s) in amounts sufficient to provide about 5% to 45%, or about 10%-30%, or about 15%-25 starch in the food products. Starchy components may include not only pure added cereal flours or other granulations but also any starchy fraction provided by other ingredients such as oat bran or soy protein. In some embodiments, a starch may comprise any conventionally employed starch or cereal flour ingredient, for use in a ready-to-eat cereal. Exemplary suitable starchy cereals include cereal flours from major cereal grains including wheat, rice, corn (maize), oats, barley, rye, or starch fractions isolated from the cereal flowers including, for example cornstarch, wheat starch, rice starch, and various treated starches including pre-gelatinized starches and/or modified starches.

In some embodiments, the food product may include a flour. Several types of flour may be used according to the disclosure. For example, common types of flour used in food products, include all-purpose flour (which like most other flour may include protein content), Whole wheat flour, Bread flour, Cake flour, pastry flour, self-rising flour, and gluten-free flour. According to the disclosure, the concentration of flour may vary depending on the intended use of a product. For example, in baked goods like bread, cakes, and cookies, flour is typically a main ingredient and may be included at concentrations ranging from 50% to 100% of the total dry ingredients. In soups and sauces, flour is often used as a thickener and may be included at concentrations ranging from 1% to 5% of the total ingredients. Batter and breading: In fried foods like chicken or fish, flour is often used as part of the batter or breading and may be included at concentrations ranging from 20% to 50% of the total dry ingredients. In snack foods, like crackers and chips, flour may be included at concentrations ranging from 30% to 70% of the total ingredients. In some embodiments, flour may comprise from about 1% by weight to about 95% by weight of a food product. In some embodiments, flours may comprise from about 1% by weight to about 95% by weight of a food product. In some embodiments, a food product may comprise flour in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a fat. In some embodiments, the food product may include an oil. In some embodiments, the food product may include one or more fats or oils. Several types of fats may be used according to the disclosure. For example, common types of fats include butter, margarine, vegetable oils, shortening, and lard. According to the disclosure, the concentration of fats may vary depending on the intended use of a product. In baked goods like cakes, cookies, and pastries, fats like butter, shortening, or oil are often used to provide moisture, flavor, and texture. In some embodiments, Fats may be included at concentrations ranging from 10% to 30% of the total ingredients. In some embodiments, like in sauces and dressings, fats like olive oil or mayonnaise are often used to provide flavor and texture. In some embodiments, Fats may be included at concentrations ranging from 10% to 30% of the total ingredients. In fried foods like chicken or French fries, fats like vegetable oil or lard are used for frying and may be included at concentrations ranging from 30% to 60% of the total ingredients. In snack foods like chips and crackers, fats like vegetable oil or palm oil are often used to provide flavor and texture. In some embodiments, Fats may be included at concentrations ranging from 10% to 30% of the total ingredients. In some embodiments, fats may comprise from about 1% by weight to about 95% by weight of a food product. In some embodiments, fats may comprise from about 1% by weight to about 95% by weight of a food product. In some embodiments, a food product may comprise fat in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include one or more additional ingredients. If desired, the disclosed food products may additionally include a variety of materials designed to improve their aesthetic or nutritional qualities. These adjuvant materials can include vitamin and/or mineral fortification, colors, flavors, sweetener(s), and mixtures thereof. The precise ingredient concentration may vary. Generally, however, such materials can each include about 0.01% to about 5%, or about 0.1% to 2% dry weight of a food product. One especially useful material is common salt. In some embodiments, salt comprises about 0.1 to 5%, or about 0.5 to 4.0% of the food products.

In some embodiments, the food product is derived from a plant. In some embodiments, the food product is derived from hemp hulls. In some embodiments, the food product is a solid food. In some embodiments, the food product is a semi-solid food. In some embodiments, the food product is a puffed product, a bakery product, a pressed cake, a cooked product, a food bar, a cereal, a crisp, or a spread.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use. In some embodiments, the composition as described herein is provided as a dietary supplement. In some embodiments, the dietary supplement is provided as a capsule, tablet, powder, or in a liquid form. In some embodiments, the composition as described herein is provided as a nutritional supplement. In some embodiments, the nutritional supplement is provided as a capsule, tablet, powder, or in a liquid form. In some embodiments, the composition as described herein is provided as a pharmaceutical. In some embodiments, the pharmaceutical is provided as a capsule, tablet, powder, or in a liquid form.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

### Methods and Uses

This disclosure provides methods and uses for treating, preventing, reducing, modulating, or ameliorating stress, symptoms of stress, or deleterious effects of chronic stress in a subject. In accordance with such methods, an effective amount of a compound or composition of this disclosure is provided to a subject in need thereof so that the subject's stress is treated, reduced, improved, restored, modulated, or ameliorated. The term “subject” as used herein refers to an animal, preferably a mammal. In some embodiments, the subject is a veterinary, companion, farm, laboratory or zoological animal. In other embodiments, the subject is a human.

In some embodiments, methods and uses for treating, preventing, reducing, modulating, or ameliorating stress disorders in a subject may include administering a therapeutically effective amount of a compound or composition as described herein. In some embodiments, administering a therapeutically effective amount of a compound includes oral ingestion of the compound. Oral ingestion of the compound may include ingesting a natural product where the compound is a naturally occurring ingredient, an enriched natural product, and a substantially pure composition of a compounds according to the disclosure. The term “stress” or “stress disorder” as used herein encompasses mental and physical diseases and conditions that are recognized and understood by physicians practicing in the relevant fields of medicine. Stress and/or stress disorders may manifest in a variety of symptoms, including but not limited to disturbed sleep, and anxiety. Stress disorders encompass a range of mental health conditions that may arise as a result of experiencing or witnessing traumatic events. Examples of such disorders include, but are not limited to, Post-Traumatic Stress Disorder (PTSD), which can develop after a person is exposed to severe trauma such as combat, natural disasters, serious accidents, or violent personal assaults. Acute Stress Disorder (ASD) is another example, presenting similar symptoms to PTSD but occurring in the immediate aftermath of the traumatic event and typically resolving within a month. Adjustment Disorder is also related, characterized by an inability to adjust to or cope with a particular stressor, like a major life change, resulting in significant emotional or behavioral symptoms. Each of these disorders manifests differently in individuals, highlighting the complex impact of stress on mental health.

As used herein, the terms “treating”, “treatment”, “treat” and the like mean affecting a subject, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. A reference to “treating” a stress disorder therefore encompasses: (a) assisting the subject to reduce stress; (b) assisting the subject remain at a lowered stress level; (c) relieving or ameliorating the effects of the stress disorder, e.g. enhancing a feeling a normalcy; or (d) preventing the stress disorder from occurring in a subject predisposed to, or at risk of, the stress disorder, so that the stress disorder does not develop or occur in the subject, or develops in a less severe form.

By “effective amount” it is meant an amount sufficient that when administered to the patient an amount of the drug is provided to achieve an effect. In the case of a therapeutic method, this effect may be the treatment of the stress disorder. Therefore, the “effective amount” may be a “therapeutically effective amount”. By “therapeutically effective amount” it is meant an amount sufficient that when administered to the subject an amount of drug is provided to treat the disease or a symptom of the disease.

In some embodiments, the present disclosure provides methods for treating, preventing, controlling, ameliorating, or reducing the risk of conditions which require potentiation of metabotropic glutamate receptor activity in a subject by administering a therapeutically effective amount of a composition as described herein. The term “potentiated amount” refers to an amount of an mGluR agonist, that is, the dosage of agonist which is effective in treating the neurological and psychiatric disorders described herein when administered in combination with an effective amount of a compound of the present disclosure. A potentiated amount is expected to be less than the amount that is required to provided the same effect when the mGluR agonist is administered without an effective amount of a compound of the present disclosure. A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with the composition as described herein, a number of factors are considered by the attending diagnostician, including, but not limited to: the mGluR agonist selected to be administered, including its potency and selectivity; the composition as described herein to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

In some embodiments, the composition as described herein may be administered as a positive allosteric modulators of the group II metabotropic glutamate receptors. In some embodiments, the composition as described herein may be administered as an agonist of a group II metabotropic glutamate receptor. In some embodiments, the composition as described herein may be administered as an antagonist of a group II metabotropic glutamate receptor.

In some embodiments, the present disclosure provides methods for treating, preventing, controlling, ameliorating, or reducing a neurological or psychiatric disorders associated with glutamate dysfunction. In some embodiments, the neurological or psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, stress, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, disorders associated with substance tolerance, disorders associated with substance withdrawal (including substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

In some embodiments, methods and uses for treating, preventing, reducing, modulating, or ameliorating a mood disorder in a subject may include administering an effective amount of a compound or composition as described herein. In some embodiments, administering a therapeutically effective amount of a compound includes oral ingestion of the compound. The term “mood disorders” as used herein encompass mental and physical diseases and conditions that are recognized and understood by physicians practicing in the relevant fields of medicine. Mood disorders may include one or more of a wide range of psychological/physiological symptoms that can significantly affect an individual's emotional state and daily functioning. Mood disorder symptoms, for example, can manifest as prolonged periods of sadness or depression, loss of interest in activities once enjoyed, significant changes in appetite or weight, sleep disturbances, fatigue, feelings of worthlessness or excessive guilt, and in severe cases, thoughts of death or suicide. Mood disorders can lead to difficulties in maintaining personal relationships, job performance, and engaging in social activities. Compounds according to the disclosure may provide natural compounds that are safe and effective in reducing anxiety. In some embodiments, compounds according to the disclosure may be more or less effective depending on the severity or type of mood disorder.

In some embodiments, methods and uses for treating, preventing, reducing, modulating, or ameliorating sleep disorders in a subject may include administering an effective amount of a compound or composition as described herein. In some embodiments, administering a therapeutically effective amount of a compound includes oral ingestion of the compound. Oral ingestion of the compound may include ingesting a natural product where the compound is a naturally occurring ingredient, an enriched natural product, and a substantially pure composition of a compounds according to the disclosure. The term “disturbed sleep” as used herein encompasses mental and physical diseases and conditions that are recognized and understood by physicians practicing in the relevant fields of medicine. Treating disturbed sleep may include addressing one or more of a broad range of symptoms that can impact quality of life and overall health. Individuals suffering from sleep disturbances may experience difficulty falling asleep (insomnia), frequent awakenings during the night, waking up too early and not being able to fall back asleep, or non-restorative sleep where one does not feel refreshed upon waking. Other symptoms include excessive daytime sleepiness, difficulty concentrating, mood changes such as irritability or increased anxiety, and decreased performance at work or school. In more severe cases, disturbed sleep can also manifest through sleep disorders like sleep apnea, characterized by abnormal breathing patterns, or restless legs syndrome, which involves uncomfortable sensations and an irresistible urge to move the legs while trying to fall asleep. Compounds according to the disclosure may provide natural compounds that are safe and effective in protecting sleep and diurnal rhythms. In some embodiments, compounds according to the disclosure may be more or less effective depending on the severity or type of disruption to sleep. In some embodiments, compounds according to the disclosure may treat sleep disorders independently from a source of stress.

In some embodiments, the compositions of the disclosure are further useful in a method for prevention, treatment, control, amelioration, or reduction of risk of the aforementioned diseases, disorders and conditions in combination with other agents, including an mGluR agonist. In some embodiments, the present disclosure provides for a method of treating, preventing, controlling, or reducing a disease or condition associated with an mGlu2 receptor by administering to the subject in need a therapeutically effective amount of a composition as described herein. In some embodiments, the present disclosure provides for a method of treating, preventing, controlling, or reducing a disease or condition associated with an mGlu3 receptor by administering to the subject in need a therapeutically effective amount of a composition as described herein.

In some embodiments, the present disclosure provides for a method for treating anxiety in a subject by administering an effective amount of the composition as described herein. In some embodiments, the anxiety is generalized anxiety disorder. In some embodiments the anxiety disorder is due to a general medical condition. In some embodiments, the anxiety is due to a substance-induced anxiety disorder. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources. In some embodiments, the subject's anxiety is improved from about 1.0%, 2.0%, 3.0%, 4.0%, 5.0%, 6.0%, 7.0%, 8.0%, 9.0%, 10.0%, 15.0%, 20%, 25.0%, 50.0%, 75.0%, or ranges including and/or spanning the aforementioned values. In some embodiments, methods and uses for treating, preventing, reducing, modulating, or ameliorating anxiety in a subject may include administering an effective amount of a compound or composition as described herein. In some embodiments, administering a therapeutically effective amount of a compound includes oral ingestion of the compound. Oral ingestion of the compound may include ingesting a natural product where the compound is a naturally occurring ingredient, an enriched natural product, and a substantially pure composition of a compounds according to the disclosure.

In another embodiment the present disclosure provides a method for treating anxiety, comprising: administering to a patient in need thereof an effective amount of a compound or composition as described herein. In some embodiments, related conditions to anxiety include, but are not limited to, panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified.

In some embodiments, the present disclosure provides for a method for treating depression brought on by stress in a subject by administering an effective amount of the composition as described herein. At present, the fourth edition of the Diagnostic and Statistical Manual of Mental Disorders (DSM-V) (2022, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder, or bipolar disorders or manic depression, for example, bipolar I disorder, bipolar II disorder and cyclothymic disorder. As used herein the term “depression” includes treatment of those depression disorders and related disorder as described in the DSM-IV. In some embodiments, the subject's depression brought on by stress is improved from about 1.0%, 2.0%, 3.0%, 4.0%, 5.0%, 6.0%, 7.0%, 8.0%, 9.0%, 10.0%, 15.0%, 20%, 25.0%, 50.0%, 75.0%, or ranges including and/or spanning the aforementioned values.

In some embodiments, the present disclosure provides for a method for treating schizophrenia in a subject by administering an effective amount of the composition as described herein. The Merck Manual (2006-2007), schizophrenia is characterized by psychosis (loss of contact with reality), hallucinations (false perceptions), delusions (false beliefs), disorganized speech and behavior, flattened affect (restricted range of emotions), cognitive deficits (impaired reasoning and problem solving), and occupational and social dysfunction. In some embodiments, the subject's schizophrenia is improved from about 1.0%, 2.0%, 3.0%, 4.0%, 5.0%, 6.0%, 7.0%, 8.0%, 9.0%, 10.0%, 15.0%, 20%, 25.0%, 50.0%, 75.0%, or ranges including and/or spanning the aforementioned values.

In some embodiments, the present disclosure provides for a method for treating a panic disorder in a subject by administering an effective amount of the composition as described herein. In some embodiments, the panic disorder is with agoraphobia. In some embodiments, the panic disorder is without agoraphobia. In some embodiments, the subject's panic disorder is improved from about 1.0%, 2.0%, 3.0%, 4.0%, 5.0%, 6.0%, 7.0%, 8.0%, 9.0%, 10.0%, 15.0%, 20%, 25.0%, 50.0%, 75.0%, or ranges including and/or spanning the aforementioned values.

In some embodiments, the present disclosure provides for a method for treating a post-traumatic stress disorder (PTSD) in a subject by administering an effective amount of the composition as described herein. In some embodiments, the subject's PTSD is improved from about 1.0%, 2.0%, 3.0%, 4.0%, 5.0%, 6.0%, 7.0%, 8.0%, 9.0%, 10.0%, 15.0%, 20%, 25.0%, 50.0%, 75.0%, or ranges including and/or spanning the aforementioned values.

The methods described herein can further include identifying a patient having a disease or condition as described herein. In some embodiments, the method further includes identifying a subject a condition which require potentiation of metabotropic glutamate receptor activity. In some embodiments, the method further includes identifying a subject with a neurological or psychiatric disorders associated with glutamate dysfunction. In some embodiments, the method further includes identifying a subject with a disease or disorder associated with an mGlu2 receptor. In some embodiments, the method further includes identifying a subject with a disease or disorder associated with an mGlu3 receptor. In some embodiments, the method further includes identifying a subject with a generalized anxiety disorder. In some embodiments, the method further includes identifying a subject with depression. In some embodiments, the method further includes identifying a subject with schizophrenia. In some embodiments, the method further includes identifying a subject with a panic disorder. In some embodiments, the method further includes identifying a subject with a post-traumatic stress disorder (PTSD). In some embodiments, identifying a subject includes collecting a sample from a patient. In some embodiments, identifying a subject includes isolating a sample from the subject. In some embodiments, isolating a sample from the subject is isolating DNA from the subject. In some embodiments, the method further includes determining whether the patient has the disease or condition.

In some embodiments, methods and uses for modulating a metabotropic glutamate receptor are provided. In some embodiments, methods and uses include providing one or more compounds of Table A or a composition including one or more compounds of Table A. In some embodiments, modulating a metabotropic glutamate receptor includes a metabotropic glutamate receptor Group II. In some embodiments, the metabotropic glutamate receptor includes one or more compounds capable of acting as an antagonist of metabotropic glutamate receptor 2 and/or metabotropic glutamate receptor 3. In some embodiments, the metabotropic glutamate receptor includes one or more compounds capable of acting as an agonist of metabotropic glutamate receptor 2 and/or metabotropic glutamate receptor 3.

In some embodiments, methods and uses of the disclosure include supporting a healthy stress response. In some embodiments, the method and uses include providing one or more compounds of Table A or a composition as described herein. In some embodiments, a composition as described herein is formulated for oral use. In some embodiments, the composition is in a dosage form configured as one of a soft-gel capsule, a hard capsule, gummy, functional beverage, functional food, liquid, powder, and a tablet. In some embodiments, the composition includes 1.0% to 99% w/w of a plant extract as described herein. In some embodiments, the composition further includes a carrier, a preservative, a sweetener a coloring agent, an additive, a flavoring agent, or a combination thereof. In some embodiments, the composition is formulated as a dietary supplement, a food ingredient or additive, a medical food, nutraceutical or pharmaceutical composition. In some embodiments, an oral composition for use in a medicament intended to support a healthy stress response or condition thereof includes one or more compounds of Table A. In some embodiments, use of a composition to support a healthy stress response in a subject, wherein the composition includes one or more compounds of Table A.

In some embodiments, methods and uses of the disclosure include supports for glutamate homeostasis. In some embodiments, the method and uses include providing one or more compounds of Table A or a composition as described herein. In some embodiments, composition as described herein is formulated for oral use. In some embodiments, the composition is in a dosage form configured as one of a soft-gel capsule, a hard capsule, gummy, functional beverage, functional food, liquid, powder, and a tablet. In some embodiments, the composition includes 1.0% to 99% w/w of a plant extract as described herein. In some embodiments, the composition further includes a carrier, a preservative, a sweetener a coloring agent, an additive, a flavoring agent, or a combination thereof. In some embodiments, the composition is formulated as a dietary supplement, a food ingredient or additive, a medical food, nutraceutical or pharmaceutical composition. In some embodiments, an oral composition for use in a medicament intended to support glutamate homeostasis or condition thereof includes one or more compounds of Table A. In some embodiments, use of a composition to support glutamate homeostasis in a subject, wherein the composition includes one or more compounds of Table A.

In some embodiments, methods and uses are provided for managing stress. In some embodiments, the method and uses include providing one or more compounds of Table A or a composition as described herein. In some embodiments, a composition as described herein is formulated for oral use. In some embodiments, the composition is in a dosage form configured as one of a soft-gel capsule, a hard capsule, gummy, functional beverage, functional food, liquid, powder, and a tablet. In some embodiments, the composition includes 1.0% to 99% w/w of a plant extract as described herein. In some embodiments, the composition further includes a carrier, a preservative, a sweetener a coloring agent, an additive, a flavoring agent, or a combination thereof. In some embodiments, the composition is formulated as a dietary supplement, a food ingredient or additive, a medical food, nutraceutical or pharmaceutical composition. In some embodiments, an oral composition for use in a medicament intended to manage stress or condition thereof includes one or more compounds of Table A. In some embodiments, use of a composition to manage stress in a subject, wherein the composition includes one or more compounds of Table A.

In some embodiments, methods and uses are provided for mood support. In some embodiments, the method and uses include providing one or more compounds of Table A or a composition as described herein. In some embodiments, a composition as described herein is formulated for oral use. In some embodiments, the composition is in a dosage form configured as one of a soft-gel capsule, a hard capsule, gummy, functional beverage, functional food, liquid, powder, and a tablet. In some embodiments, the composition includes 1.0% to 99% w/w of a plant extract as described herein. In some embodiments, the composition further includes a carrier, a preservative, a sweetener a coloring agent, an additive, a flavoring agent, or a combination thereof. In some embodiments, the composition is formulated as a dietary supplement, a food ingredient or additive, a medical food, nutraceutical or pharmaceutical composition. In some embodiments, an oral composition for use in a medicament intended to provide mood support or improve a condition thereof includes one or more compounds of Table A. In some embodiments, use of a composition to provide mood support in a subject, wherein the composition includes one or more compounds of Table A.

### Accordingly, Some Aspects Described Herein Relate to the Following Numbered Alternatives:

1. An oral composition for treating, reducing, modulating, or ameliorating stress, symptoms of stress, or deleterious effects of chronic stress, the oral composition comprising: a therapeutically effective amount of a plant extract; and one or more carriers or excipients, wherein, the therapeutically effective amount of the plant extract is capable of treating, reducing, modulating, or ameliorating stress, symptoms of stress, or deleterious effects of chronic stress.
2. The oral composition of alternative 1, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
3. The oral composition of alternative 1 or 2, wherein the oral composition is formulated as a dietary supplement, food ingredient or additive, a medical food, nutraceutical or pharmaceutical composition.
4. The oral composition of any one of alternatives 1 to 3, wherein the oral composition is in a dosage form configured as one of a soft-gel capsule, a hard capsule, liquid, powder, and a tablet.
5. The oral compositions of any one of alternatives 1 to 4, wherein the oral composition comprises 10% to 99% w/w of the plant extract.
6. The oral composition of any one of alternatives 1 to 5, wherein the plant extract further comprises a preservative, sweetener, coloring agent, an additive, a flavoring agent, or a combination thereof.
7. A method of treating, preventing, reducing, modulating, or ameliorating stress, symptoms of stress, or deleterious effects of chronic stress in a subject, in need thereof, the method comprising administering a therapeutically effective amount of a composition comprising the oral composition of any one of alternatives 1 to 6.
8. The method of alternative 7, wherein the plant extract of the oral composition is capable of activating a group II metabotropic receptor.
9. The method of alternative 7 or 8, wherein the plant extract activates an mGlu2 receptor.
10. The method of alternative 7 or 8, wherein the plant extract activates an mGlu3 receptor.
11. An oral composition, comprising: a plant extract which is a mGlu2 or mGlu3 receptor antagonist; and one or more carriers or excipients.
12. The oral composition of alternative 11, wherein the plant extract is selected from the group consisting of Rheum officinale, Phyllanthus emblica, Theobroma cacao, or a combination thereof.
13. The oral composition of alternative 11 or 12, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
14. The oral composition of any one of alternatives 11 to 13, wherein the oral composition is formulated as a dietary supplement, food ingredient or additive, a medical food, nutraceutical or pharmaceutical composition.
15. The oral composition of any one of alternatives 11 to 14, wherein the oral composition is in a dosage form configured as one of a soft-gel capsule, a hard capsule, liquid, powder, and a tablet.
16. The oral compositions of any one of alternatives 11 to 15, wherein the oral composition comprises 10% to 99% w/w of the plant extract.
17. The oral composition of any one of alternatives 11 to 16, wherein the plant extract further comprises a preservative, a sweetener, a coloring agent, an additive, a flavoring agent, or a combination thereof.
18. An oral composition, comprising: a plant extract which is a mGlu2 or mGlu3 receptor agonist or positive allosteric modulator; and one or more carriers or excipients.
19. The oral composition of alternative 18, wherein the plant extract is selected from the group consisting of Tripterygium wilfordii, Phyllanthus emblica, Hypericum perforatum, Aloe barbadensis, Hypericum perforatum, or a combination thereof.
20. The oral composition of alternative 18 or 19, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
21. The oral composition of any one of alternatives 18 to 20, wherein the oral composition is formulated as a dietary supplement, food ingredient or additive, a medical food, nutraceutical or pharmaceutical composition.
22. The oral composition of any one of alternatives 18 to 21, wherein the oral composition is in a dosage form configured as one of a soft-gel capsule, a hard capsule, liquid, powder, and a tablet.
23. The oral compositions of any one of alternatives 18 to 22, wherein the oral composition comprises 10% to 99% w/w of the plant extract.
24. The oral composition of any one of alternatives 18 to 23, wherein the plant extract further comprises a preservative, a sweetener, a coloring agent, an additive, a flavoring agent, or a combination thereof.
25. A method of modulating a disease or condition associated with mGlu2/3, comprising: administering to a subject in need thereof a therapeutically effective amount of the oral composition of any one of alternatives 18 to 24.
26. An oral composition for inhibiting mGlu2 or mGlu3, the composition comprising: a plant extract selected from the group consisting of Rheum officinale, Theobroma cacao, Hordeum vulgare cv. Seco, Chaenomeles sinensis, Nepenthes x hookeriana, Musa acuminata, Pseudotsuga menziesii var. menziesii, Penstemon centranthifolius, Pisum sativum, Aloe Africana, Glycine max, Avena sativa, Malus domestica, Gardenia jasminoides, Aloe ferox, Oryza sativa, Coffea arabica, Carthamus tinctorius, Macrotyloma uniflorum, Solanum melongena, Aloe vera, Citrus unshiu, Hypericum perforatum, Curcuma longa, Nypa fruticans, Secale cereale cv. Aroostook, Persicaria tinctoria, Cannabis sativa, Solanum lycopersicum, Citrus clementina, Trifolium pratense cv. Redman, Uncaria tomentosa, Auricularia auricula-judae, Citrus sinensis, Cycas revoluta, Momordica charantia, Brassica carinata, Gaillardia pulchella, Nelumbo nucifera, Raphanus sativus var. niger, Lepidium meyenii, Zea mays, Nicotiana tabacum, Agaricus subrufescens, Linum usitatissimum, Paeonia lactiflora, Capsicum annuum, Larrea tridentata, Mirabilis jalapa, Catharanthus roseus, or a combination thereof; and one or more carriers or excipients.
27. The oral composition of alternative 26, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
28. The oral composition of alternative 26 or 27, wherein the oral composition is formulated as a dietary supplement, food ingredient or additive, a medical food, nutraceutical or pharmaceutical composition.
29. The oral composition of any one of alternatives 26 to 28, wherein the oral composition is in a dosage form configured as one of a soft-gel capsule, a hard capsule, liquid, powder, and a tablet.
30. The oral compositions of any one of alternatives 26 to 29, wherein the oral composition comprises 10% to 99% w/w of the plant extract.
31. The oral composition of any one of alternatives 26 to 30, wherein the plant extract further comprises a preservative, a sweetener, a coloring agent, an additive, a flavoring agent, or a combination thereof.
32. A method of treating or modulating a disease or condition associated with mGlu2 mGlu3, comprising: administering to a subject in need thereof a therapeutically effective amount of the oral composition of any one of alternatives 26 to 31.
33. An oral composition for activating mGlu2 or mGlu3, the composition comprising: a plant extract selected from the group consisting of Momordica charantia, Aloe vera, Silybum marianum, Aloe ferox, Rheum officinale, Chaenomeles sinensis, Citrus clementina, Hordeum vulgare cv. Seco, Erucastrum gallicum, Linum usitatissimum, Asclepias syriaca, Hypericum perforatum, Larrea tridentata, Eucalyptus globulus subsp. bicostata, Phyllanthus emblica, Veronica polita, Alcea rosea, Neolamarckia cadamba, Catharanthus roseus, Podocarpus spicatus, Tripterygium wilfordii, Aloe africana, Juniperus monosperma, Pleurotus ostreatus, Gypsophila perfoliate, Clausena excavate, Gynura procumbens, Trifolium pratense cv. Redman, Rosmarinus officinalis, Nelumbo nucifera, Citrus unshiu, Hylocomium splendens, Citrus sinensis, Juniperus drupacea, Forsythia suspensa, or combinations thereof; and one or more carriers or excipients.
34. The oral composition of alternative 33, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
35. The oral composition of alternative 33 or 34, wherein the oral composition is formulated as a dietary supplement, food ingredient or additive, a medical food, nutraceutical or pharmaceutical composition.
36. The oral composition of any one of alternatives 33 to 35, wherein the oral composition is in a dosage form configured as one of a soft-gel capsule, a hard capsule, liquid, powder, and a tablet.
37. The oral compositions of any one of alternatives 33 to 36, wherein the oral composition comprises 10% to 99% w/w of the plant extract.
38. The oral composition of any one of alternatives 33 to 37, wherein the plant extract further comprises a preservative, a sweetener, a coloring agent, an additive, a flavoring agent, or a combination thereof.
39. A method of treating or modulating a disease or condition associated with mGlu2 or mGlu3, comprising: administering to a subject in need thereof a therapeutically effective amount of the oral composition of any one of alternatives 33 to 38.
40. An oral composition with enhanced binding affinity with increased activity, the composition comprising: a plant extract selected from the group consisting of Medicago sativa subsp. sativa, Solanum Americanum, Coffea arabica, Momordica charantia, Aloe vera, Silybum marianum, Theobroma cacao, Aloe ferox, Gardenia jasminoides, Anacardium occidentale, Capsicum annuum, Paeonia lactiflora, Rheum officinale, Chaenomeles sinensis, Citrus clementina, Hordeum vulgare cv. Seco, Erucastrum gallicum, Linum usitatissimum, Asclepias syriaca, Avena sativa, Hypericum perforatum, Curcuma longa, Zea mays, Larrea tridentata, Nicotiana tabacum, Eucalyptus globulus subsp. bicostata, Musa acuminata, Solanum melongena, Phyllanthus emblica, Solanum lycopersicum, Veronica polita, Alcea rosea, Plukenetia volubilis, Euphorbia tirucalli, Neolamarckia cadamba, Citrus limon, Catharanthus roseus, Mirabilis jalapa, Podocarpus spicatus, Tripterygium wilfordii, Spatholobus suberectus, Aloe africana, Crinum moorei, Juniperus monosperma, Pleurotus ostreatus, Gypsophila perfoliate, Clausena excavate, Gynura procumbens, Trifolium pratense cv. Redman, Oryza sativa, Rosmarinus officinalis, Nelumbo nucifera, Carthamus tinctorius, Citrus unshiu, Hylocomium splendens, Citrus sinensis, Juniperus drupacea, Forsythia suspensa, or combinations thereof; and one or more carriers or excipients.
41. The oral composition of alternative 40, wherein the plant extract comprises a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof.
42. The oral composition of alternative 40 or 41, wherein the oral composition is formulated as a dietary supplement, food ingredient or additive, a medical food, nutraceutical or pharmaceutical composition.
43. The oral composition of any one of alternatives 40 to 42, wherein the oral composition is in a dosage form configured as one of a soft-gel capsule, a hard capsule, liquid, powder, and a tablet.
44. The oral compositions of any one of alternatives 40 to 43, wherein the oral composition comprises 10% to 99% w/w of the plant extract.
45. The oral composition of any one of alternatives Error! Reference source not found. to Error! Reference source not found, wherein the plant extract further comprises a preservative, a sweetener, a coloring agent, an additive, a flavoring agent, or a combination thereof.
46. A method of treating or modulating a disease or condition associated with mGlu2 or mGlu3, comprising: administering to a subject in need thereof a therapeutically effective amount of the oral composition of any one of alternatives 40 to 45.
47. A method of treating, preventing, reducing, modulating, or ameliorating anxiety in a subject, the method comprising: providing to the subject a composition comprising a compound selected from a mGlu2 receptor antagonist, a mGlu3 antagonist, or a combination thereof; and a carrier.
48. A method of treating, preventing, reducing, modulating, or ameliorating anxiety in a subject, the method comprising: providing to the subject a composition comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof; and a carrier.
49. A method of treating, preventing, reducing, modulating, or ameliorating a negative effect of emotional stress on sleep in a subject, the method comprising: providing to the subject a composition comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof; and a carrier.
50. A method of treating, preventing, reducing, modulating, or ameliorating a negative effect of emotional stress on sleep in a subject, the method comprising: providing to the subject a composition comprising a compound selected from a mGlu2 receptor antagonist, a mGlu3 antagonist, or a combination thereof; and a carrier.
51. A method of treating, preventing, reducing, modulating, or ameliorating a negative effect of physiological rhythms in a subject, the method comprising: providing to the subject a composition comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof; and a carrier.
52. A method of treating, preventing, reducing, modulating, or ameliorating a negative effect of physiological rhythms in a subject, the method comprising: providing to the subject a composition comprising a compound selected from a mGlu2 receptor antagonist, a mGlu3 antagonist, or a combination thereof; and a carrier.
53. A method of treating, preventing, reducing, modulating, or ameliorating a negative effect of mood in a subject, the method comprising: providing to the subject a composition comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof; and a carrier.
54. A method of treating, preventing, reducing, modulating, or ameliorating a negative effect of mood in a subject, the method comprising: providing to the subject a composition comprising a compound selected from a mGlu2 receptor antagonist, a mGlu3 antagonist, or a combination thereof; and a carrier.
55. A method of treating, preventing, reducing, modulating, or ameliorating disturbed sleep in a subject, the method comprising: providing to the subject a composition comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof; and a carrier.
56. An oral composition for use in a medicament intended for the treatment of stress, anxiety, disturbed sleep, or condition thereof comprising a compound selected from the group consisting of (2S)-2alpha-(4-Hydroxyphenyl)-2H-1-benzopyran-7-ol, guibourtinidol, Frangula-emodin, Aloe-emodin, (3aR,4aR,7aR,8R,9aR)-4a,8-dimethyl-3-methylene-2,5-dioxo-2,3,3a,4,4a,5,7a,8,9,9a-decahydroazuleno[6,5-b]furan-4-yl methacrylate, or a combination thereof and a carrier.

### EXAMPLES

The following examples demonstrate protocols and methods to validate and demonstrate the activity of compounds of the disclosure towards treating or ameliorating one or more conditions described herein. In some examples, the experiments may demonstrate or evaluate whether one or more of the compounds of the disclosure reduces the negative effects of emotional stress on sleep, physiological rhythms, and mood. One or more compounds of the disclosure may be administered to a subject via oral ingestion, such as in the form of a nutraceutical supplement. In some embodiments, methods and procedures according to the following examples may be performed on a rodent, such as a mouse or a rat. If applicable, an experiment to test the efficacy of oral supplements in reducing the effect of stress on rodents, may be designed and conducted under the supervision of an Animal Care and Use Committee (ACUC). Such as committee may assist in ensuring that research protocols involving animals are conducted in compliance with ethical standards, minimizing discomfort, distress, and pain to the animals. Depending on the compound under investigation, rigorous in vivo preclinical testing should be performed before clinical testing. Such in vivo preclinical testing can, as described below, include validated rat models of emotional stress using in vivo biotelemetry tools and infrared video home cage surveillance.

### Example 1

Dionex™ accelerated solvent extractor 350 was used for the extraction of plant material. Four 100 mL stainless steel ASE extraction cells were filled with 25 g of grinded plant tissues. The pre-set temperature was 60° C., the static extraction was run for a total of twelve extraction cycles. The solvents used for the extraction were 95% ethanol, ethyl acetate, and hexane. The solvent was removed, and the 95% ethanol extract was subjected to a bioassay-guided fractionation protocol. Briefly, the extract was dry-loaded onto C18 spherical flash column chromatography. Elution was conducted with a mobile phase consisting of water+0.1% formic acid (A) and CH₃CN+0.1% formic acid (B), following the gradient, 1% to 3% B in 3 min, increasing to 100% B in 22 min then maintaining 100% B for 7 min at a flow rate of 30 mL/min. The highly active fractions were pooled, and the polled sample was further purified by bioassay-guided fractionation using Kinetex reverse-phase C₁₈ preparative column yielding guibourtinidol.

### Example 2

This is a prophetic example. In this example, the extraction method of Example 1 to the plant tissues from Table 1.

TABLE 1

| Scientific Name | Plant Tissue(s) |
|---|---|
| Abri herba | Leaf, pulp, fruit, bulb, seed, |
| | tuber, root, flower |
| Abrus pulchellus subsp. | Leaf, pulp, fruit, bulb, seed, |
| cantoniensis | tuber, root, flower |
| Acacia luederitzii | Leaf, pulp, fruit, bulb, seed, |
| | tuber, root, flower |
| Achyranthes bidentata | Leaf, pulp, fruit, bulb, seed, |
| | tuber, root, flower |
| Aconitum seravschanicum | Leaf, pulp, fruit, bulb, seed, |
| | tuber, root, flower |
| Aconitum tasiromontanum | Leaf, pulp, fruit, bulb, seed, |
| | tuber, root, flower |
| Acori tataninowii rhizoma | Leaf, pulp, fruit, bulb, seed, |
| | tuber, root, flower |
| Acorus gramineus | Leaf, pulp, fruit, bulb, seed, |
| | tuber, root, flower |
| Acorus tatarinowii | Leaf, pulp, fruit, bulb, seed, |
| | tuber, root, flower |
| Actinidia chinensis planch | Leaf, pulp, fruit, bulb, seed, |
| | tuber, root, flower |
| Adansonia grandidieri | Leaf, pulp, fruit, bulb, seed, |
| | tuber, root, flower |
| Aegle marmelos | Leaf, pulp, fruit, bulb, seed, |
| | tuber, root, flower |
| Aframomum giganteum | Leaf, pulp, fruit, bulb, seed, |
| | tuber, root, flower |
| Agaricus subrufescens | Leaf, pulp, fruit, bulb, seed, |
| | tuber, root, flower |
| Aizopsis selskiana | Leaf, pulp, fruit, bulb, seed, |
| | tuber, root, flower |
| Albizia myriophylla | Leaf, pulp, fruit, bulb, seed, |
| | tuber, root, flower |
| Alcea rosea | Leaf, pulp, fruit, bulb, seed, |
| | tuber, root, flower |
| Alisma plantago-aquatica | Leaf, pulp, fruit, bulb, seed, |
| subsp. orientale | tuber, root, flower |
| Aloe africana | Leaf, pulp, fruit, bulb, seed, |
| | tuber, root, flower |
| Aloe arborescens | Leaf, pulp, fruit, bulb, seed, |
| | tuber, root, flower |
| Aloe barbadensis | Leaf, pulp, fruit, bulb, seed, |
| | tuber, root, flower |
| Aloe castellorum | Leaf, pulp, fruit, bulb, seed, |
| | tuber, root, flower |
| Aloe elgonica | Leaf, pulp, fruit, bulb, seed, |
| | tuber, root, flower |
| Aloe ferox | Leaf, pulp, fruit, bulb, seed, |
| | tuber, root, flower |
| Aloe rubroviolacea | Leaf, pulp, fruit, bulb, seed, |
| | tuber, root, flower |
| Aloe spicata | Leaf, pulp, fruit, bulb, seed, |
| | tuber, root, flower |
| Aloe succotrina | Leaf, pulp, fruit, bulb, seed, |
| | tuber, root, flower |
| Aloe vera | Leaf, pulp, fruit, bulb, seed, |
| | tuber, root, flower |
| Alpiniae officinarum rhizome | Leaf, pulp, fruit, bulb, seed, |
| | tuber, root, flower |
| Amaranthus hypochondriacus | Leaf, pulp, fruit, bulb, seed, |
| | tuber, root, flower |
| Ampelopsis japonica | Leaf, pulp, fruit, bulb, seed, |
| | tuber, root, flower |
| Amphorogyne spicata | Leaf, pulp, fruit, bulb, seed, |
| | tuber, root, flower |
| Anacardium occidentale | Leaf, pulp, fruit, bulb, seed, |
| | tuber, root, flower |
| Anaphalis lactea | Leaf, pulp, fruit, bulb, seed, |
| | tuber, root, flower |
| Anchusa azurea | Leaf, pulp, fruit, bulb, seed, |
| | tuber, root, flower |
| Andrographis laxiflora | Leaf, pulp, fruit, bulb, seed, |
| | tuber, root, flower |
| Andrographis neesiana | Leaf, pulp, fruit, bulb, seed, |
| | tuber, root, flower |
| Anemarrhena asphodeloides | Leaf, pulp, fruit, bulb, seed, |
| | tuber, root, flower |
| Angelica archangelica | Leaf, pulp, fruit, bulb, seed, |
| | tuber, root, flower |
| Anthema tinctoria | Leaf, pulp, fruit, bulb, seed, |
| | tuber, root, flower |
| Aphyllocladus denticulatus | Leaf, pulp, fruit, bulb, seed, |
| | tuber, root, flower |
| Aralia decaisneana | Leaf, pulp, fruit, bulb, seed, |
| | tuber, root, flower |
| Arnica acaulis | Leaf, pulp, fruit, bulb, seed, |
| | tuber, root, flower |
| Arnica montana | Leaf, pulp, fruit, bulb, seed, |
| | tuber, root, flower |
| Artemisia annua | Leaf, pulp, fruit, bulb, seed, |
| | tuber, root, flower |
| Artemisia parviflora | Leaf, pulp, fruit, bulb, seed, |
| | tuber, root, flower |
| Artemisia scoparia [Syn. | Leaf, pulp, fruit, bulb, seed, |
| Artemisia capillaris v | tuber, root, flower |
| Asclepias syriaca | Leaf, pulp, fruit, bulb, seed, |
| | tuber, root, flower |
| Asphodeline tenuior | Leaf, pulp, fruit, bulb, seed, |
| | tuber, root, flower |
| Asphodelus aestivus | Leaf, pulp, fruit, bulb, seed, |
| | tuber, root, flower |
| Asphodelus albus | Leaf, pulp, fruit, bulb, seed, |
| | tuber, root, flower |
| Asphodelus fistulosus | Leaf, pulp, fruit, bulb, seed, |
| | tuber, root, flower |
| Asphodelus ramosus | Leaf, pulp, fruit, bulb, seed, |
| | tuber, root, flower |
| Asphodelus tenuifolius | Leaf, pulp, fruit, bulb, seed, |
| | tuber, root, flower |
| Aspidosperma discolor | Leaf, pulp, fruit, bulb, seed, |
| | tuber, root, flower |
| Aster sedifolius | Leaf, pulp, fruit, bulb, seed, |
| | tuber, root, flower |
| Aster tataricus | Leaf, pulp, fruit, bulb, seed, |
| | tuber, root, flower |
| Asteris radix et rhizoma | Leaf, pulp, fruit, bulb, seed, |
| | tuber, root, flower |
| Atadinus libanoticus | Leaf, pulp, fruit, bulb, seed, |
| | tuber, root, flower |
| Auricularia auricula-judae | Leaf, pulp, fruit, bulb, seed, |
| | tuber, root, flower |
| Avena sativa | Leaf, pulp, fruit, bulb, seed, |
| | tuber, root, flower |
| Baccharis potosina | Leaf, pulp, fruit, bulb, seed, |
| | tuber, root, flower |
| Balanophora laxiflora | Leaf, pulp, fruit, bulb, seed, |
| | tuber, root, flower |
| Bassia muricata | Leaf, pulp, fruit, bulb, seed, |
| | tuber, root, flower |
| Bauhinia manca | Leaf, pulp, fruit, bulb, seed, |
| | tuber, root, flower |
| Benincasa hispida | Leaf, pulp, fruit, bulb, seed, |
| | tuber, root, flower |
| Berchemia floribunda | Leaf, pulp, fruit, bulb, seed, |
| | tuber, root, flower |
| Bergenia hissarica | Leaf, pulp, fruit, bulb, seed, |
| | tuber, root, flower |
| Beta vulgaris vulgaris | Leaf, pulp, fruit, bulb, seed, |
| | tuber, root, flower |
| Bistorta elliptica | Leaf, pulp, fruit, bulb, seed, |
| | tuber, root, flower |
| Bistorta officinalis | Leaf, pulp, fruit, bulb, seed, |
| | tuber, root, flower |
| Boehmeria nivea | Leaf, pulp, fruit, bulb, seed, |
| | tuber, root, flower |
| Boehmeria platanifolia [Syn. | Leaf, pulp, fruit, bulb, seed, |
| Boehmeria tricuspis | tuber, root, flower |
| Boehmeriae rhizoma et radix | Leaf, pulp, fruit, bulb, seed, |
| | tuber, root, flower |
| Bolbostemae paniculati bulbus | Leaf, pulp, fruit, bulb, seed, |
| | tuber, root, flower |
| Bolbostemma paniculatum | Leaf, pulp, fruit, bulb, seed, |
| | tuber, root, flower |
| Botrytis cinerea | Leaf, pulp, fruit, bulb, seed, |
| | tuber, root, flower |
| Brassica carinata | Leaf, pulp, fruit, bulb, seed, |
| | tuber, root, flower |
| Brassica rapa | Leaf, pulp, fruit, bulb, seed, |
| | tuber, root, flower |
| Bridelia stipularis | Leaf, pulp, fruit, bulb, seed, |
| | tuber, root, flower |
| Brosimum acutifolium | Leaf, pulp, fruit, bulb, seed, |
| | tuber, root, flower |
| broussonetia kanzinoki | Leaf, pulp, fruit, bulb, seed, |
| | tuber, root, flower |
| Broussonetia kazinoki | Leaf, pulp, fruit, bulb, seed, |
| | tuber, root, flower |
| Broussonetia papyrifera | Leaf, pulp, fruit, bulb, seed, |
| | tuber, root, flower |
| Bryophyllum pinnatum | Leaf, pulp, fruit, bulb, seed, |
| | tuber, root, flower |
| Bulbine annua | Leaf, pulp, fruit, bulb, seed, |
| | tuber, root, flower |
| Bulbine asphodeloides | Leaf, pulp, fruit, bulb, seed, |
| | tuber, root, flower |
| Callisia fragrans | Leaf, pulp, fruit, bulb, seed, |
| | tuber, root, flower |
| Canarium bengalense | Leaf, pulp, fruit, bulb, seed, |
| | tuber, root, flower |
| Cannabis sativa | Leaf, pulp, fruit, bulb, seed, |
| | tuber, root, flower |
| Capsicum annuum | Leaf, pulp, fruit, bulb, seed, |
| | tuber, root, flower |
| Carduus tenuiflorus | Leaf, pulp, fruit, bulb, seed, |
| | tuber, root, flower |
| Carthamus tinctorius | Leaf, pulp, fruit, bulb, seed, |
| | tuber, root, flower |
| Carthamus tinctorius | Leaf, pulp, fruit, bulb, seed, |
| | tuber, root, flower |
| Cascara sagrada | Leaf, pulp, fruit, bulb, seed, |
| | tuber, root, flower |
| Cassia abbreviata | Leaf, pulp, fruit, bulb, seed, |
| | tuber, root, flower |
| Cassia alata L. | Leaf, pulp, fruit, bulb, seed, |
| | tuber, root, flower |
| Cassia aldabrensis | Leaf, pulp, fruit, bulb, seed, |
| | tuber, root, flower |
| Cassia angolensis | Leaf, pulp, fruit, bulb, seed, |
| | tuber, root, flower |
| Cassia fastuosa | Leaf, pulp, fruit, bulb, seed, |
| | tuber, root, flower |
| Cassia fistula | Leaf, pulp, fruit, bulb, seed, |
| | tuber, root, flower |
| Cassia grandis | Leaf, pulp, fruit, bulb, seed, |
| | tuber, root, flower |
| Cassia javanica | Leaf, pulp, fruit, bulb, seed, |
| | tuber, root, flower |
| Cassia obtusifolia | Leaf, pulp, fruit, bulb, seed, |
| | tuber, root, flower |
| Cassia occidentalis | Leaf, pulp, fruit, bulb, seed, |
| | tuber, root, flower |
| Cassia speciosa | Leaf, pulp, fruit, bulb, seed, |
| | tuber, root, flower |
| Cassia tora | Leaf, pulp, fruit, bulb, seed, |
| | tuber, root, flower |
| Cassia tora L. | Leaf, pulp, fruit, bulb, seed, |
| | tuber, root, flower |
| Cassia torosa L. | Leaf, pulp, fruit, bulb, seed, |
| | tuber, root, flower |
| Cassiae semen | Leaf, pulp, fruit, bulb, seed, |
| | tuber, root, flower |
| Catalpa longissima | Leaf, pulp, fruit, bulb, seed, |
| | tuber, root, flower |
| Catharanthus roseu | Leaf, pulp, fruit, bulb, seed, |
| | tuber, root, flower |
| Catharanthus roseus | Leaf, pulp, fruit, bulb, seed, |
| | tuber, root, flower |
| Caulerpa peltata | Leaf, pulp, fruit, bulb, seed, |
| | tuber, root, flower |
| Caulis polygoni multiflori | Leaf, pulp, fruit, bulb, seed, |
| | tuber, root, flower |
| Caulis sargentodoxae | Leaf, pulp, fruit, bulb, seed, |
| | tuber, root, flower |
| Caulocystis cephalornithos | Leaf, pulp, fruit, bulb, seed, |
| | tuber, root, flower |
| Centipeda minima | Leaf, pulp, fruit, bulb, seed, |
| | tuber, root, flower |
| Cephaelis ipecacuanha | Leaf, pulp, fruit, bulb, seed, |
| | tuber, root, flower |
| Cephalotaxus griffithii | Leaf, pulp, fruit, bulb, seed, |
| | tuber, root, flower |
| Chaenomeles sinensis | Leaf, pulp, fruit, bulb, seed, |
| | tuber, root, flower |
| Chaiturus marrubiastrum | Leaf, pulp, fruit, bulb, seed, |
| | tuber, root, flower |
| Chamaecrista mimosoides | Leaf, pulp, fruit, bulb, seed, |
| | tuber, root, flower |
| Chamaecrista nigricans | Leaf, pulp, fruit, bulb, seed, |
| | tuber, root, flower |
| Chamaecrista nomame | Leaf, pulp, fruit, bulb, seed, |
| | tuber, root, flower |
| Chamaecrista pumila | Leaf, pulp, fruit, bulb, seed, |
| | tuber, root, flower |
| Cheilanthes argentea | Leaf, pulp, fruit, bulb, seed, |
| | tuber, root, flower |
| Chenopodium quinoa | Leaf, pulp, fruit, bulb, seed, |
| | tuber, root, flower |
| Cichorium intybus | Leaf, pulp, fruit, bulb, seed, |
| | tuber, root, flower |
| Cirsium oligophyllum | Leaf, pulp, fruit, bulb, seed, |
| | tuber, root, flower |
| Citrus clementina | Leaf, pulp, fruit, bulb, seed, |
| | tuber, root, flower |
| Citrus limon | Leaf, pulp, fruit, bulb, seed, |
| | tuber, root, flower |
| Citrus sinensis | Leaf, pulp, fruit, bulb, seed, |
| | tuber, root, flower |
| Citrus unshiu | Leaf, pulp, fruit, bulb, seed, |
| | tuber, root, flower |
| Clausena excavate | Leaf, pulp, fruit, bulb, seed, |
| | tuber, root, flower |
| Clibadium sprucei | Leaf, pulp, fruit, bulb, seed, |
| | tuber, root, flower |
| Coffea arabica | Leaf, pulp, fruit, bulb, seed, |
| | tuber, root, flower |
| Colophospermum mopane | Leaf, pulp, fruit, bulb, seed, |
| | tuber, root, flower |
| Commiphora socotrana | Leaf, pulp, fruit, bulb, seed, |
| | tuber, root, flower |
| Coreopsis tinctoria | Leaf, pulp, fruit, bulb, seed, |
| | tuber, root, flower |
| Corydalis rhizoma | Leaf, pulp, fruit, bulb, seed, |
| | tuber, root, flower |
| Cranfillia fluviatilis | Leaf, pulp, fruit, bulb, seed, |
| | tuber, root, flower |
| Cratoxylum formosum | Leaf, pulp, fruit, bulb, seed, |
| | tuber, root, flower |
| Cremastra appendiculata | Leaf, pulp, fruit, bulb, seed, |
| | tuber, root, flower |
| Crinum moorei | Leaf, pulp, fruit, bulb, seed, |
| | tuber, root, flower |
| Curcuma longa | Leaf, pulp, fruit, bulb, seed, |
| | tuber, root, flower |
| Curcuma longa | Leaf, pulp, fruit, bulb, seed, |
| | tuber, root, flower |
| Cycas revoluta | Leaf, pulp, fruit, bulb, seed, |
| | tuber, root, flower |
| Cymbopogon spp | Leaf, pulp, fruit, bulb, seed, |
| | tuber, root, flower |
| Cynanchum viminale | Leaf, pulp, fruit, bulb, seed, |
| | tuber, root, flower |
| Dalbergia hupeana | Leaf, pulp, fruit, bulb, seed, |
| | tuber, root, flower |
| Dalbergia lanceolaria | Leaf, pulp, fruit, bulb, seed, |
| subsp. paniculata | tuber, root, flower |
| Dalbergia latifolia | Leaf, pulp, fruit, bulb, seed, |
| | tuber, root, flower |
| Daucus carota | Leaf, pulp, fruit, bulb, seed, |
| | tuber, root, flower |
| Degenia velebitica | Leaf, pulp, fruit, bulb, seed, |
| | tuber, root, flower |
| Delphinium nuttallianum | Leaf, pulp, fruit, bulb, seed, |
| | tuber, root, flower |
| Delphinium virgatum | Leaf, pulp, fruit, bulb, seed, |
| | tuber, root, flower |
| Dendriopoterium menendezii | Leaf, pulp, fruit, bulb, seed, |
| | tuber, root, flower |
| Dendrobium thyrsiflorum | Leaf, pulp, fruit, bulb, seed, |
| | tuber, root, flower |
| Derris canarensis | Leaf, pulp, fruit, bulb, seed, |
| | tuber, root, flower |
| Derris cuneifolia | Leaf, pulp, fruit, bulb, seed, |
| | tuber, root, flower |
| Derris oblonga | Leaf, pulp, fruit, bulb, seed, |
| | tuber, root, flower |
| Dianthi herba | Leaf, pulp, fruit, bulb, seed, |
| | tuber, root, flower |
| Dianthus caryophyllus | Leaf, pulp, fruit, bulb, seed, |
| | tuber, root, flower |
| Dictyopteris australis | Leaf, pulp, fruit, bulb, seed, |
| | tuber, root, flower |
| Dillenia indica | Leaf, pulp, fruit, bulb, seed, |
| | tuber, root, flower |
| Dioscoreae bulbiferae | Leaf, pulp, fruit, bulb, seed, |
| | tuber, root, flower |
| Dipsacus azureus | Leaf, pulp, fruit, bulb, seed, |
| | tuber, root, flower |
| Dodonaea viscosa | Leaf, pulp, fruit, bulb, seed, |
| | tuber, root, flower |
| Dracaena cambodiana | Leaf, pulp, fruit, bulb, seed, |
| | tuber, root, flower |
| Dracaena draco | Leaf, pulp, fruit, bulb, seed, |
| | tuber, root, flower |
| Dysoxylum cumingianum | Leaf, pulp, fruit, bulb, seed, |
| | tuber, root, flower |
| Dysoxylum muelleri | Leaf, pulp, fruit, bulb, seed, |
| | tuber, root, flower |
| Elymus repens | Leaf, pulp, fruit, bulb, seed, |
| | tuber, root, flower |
| Enceliopsis argophylla | Leaf, pulp, fruit, bulb, seed, |
| | tuber, root, flower |
| endophytic aspergillus | Leaf, pulp, fruit, bulb, seed, |
| | tuber, root, flower |
| Epimedium koreanum | Leaf, pulp, fruit, bulb, seed, |
| | tuber, root, flower |
| Epimrdii herba | Leaf, pulp, fruit, bulb, seed, |
| | tuber, root, flower |
| Eria mollis | Leaf, pulp, fruit, bulb, seed, |
| | tuber, root, flower |
| Eriocaulon buergerianum | Leaf, pulp, fruit, bulb, seed, |
| | tuber, root, flower |
| Eriocaulon sieboldianum | Leaf, pulp, fruit, bulb, seed, |
| | tuber, root, flower |
| Erucastrum gallicum | Leaf, pulp, fruit, bulb, seed, |
| | tuber, root, flower |
| Erythrina lysistemon | Leaf, pulp, fruit, bulb, seed, |
| | tuber, root, flower |
| Erythrophleum ivorense | Leaf, pulp, fruit, bulb, seed, |
| | tuber, root, flower |
| Eucalyptus globulus | Leaf, pulp, fruit, bulb, seed, |
| subsp. bicostata | tuber, root, flower |
| Eucalyptus globulus | Leaf, pulp, fruit, bulb, seed, |
| subsp. bicostata | tuber, root, flower |
| Euodia bodinieri | Leaf, pulp, fruit, bulb, seed, |
| | tuber, root, flower |
| Euodia officinalis | Leaf, pulp, fruit, bulb, seed, |
| | tuber, root, flower |
| Euodia ruticarpa | Leaf, pulp, fruit, bulb, seed, |
| | tuber, root, flower |
| Euphorbia tirucalli | Leaf, pulp, fruit, bulb, seed, |
| | tuber, root, flower |
| Fagopyrum esculentum | Leaf, pulp, fruit, bulb, seed, |
| | tuber, root, flower |
| Fagopyrum tataricum | Leaf, pulp, fruit, bulb, seed, |
| | tuber, root, flower |
| Fallopia multiflora | Leaf, pulp, fruit, bulb, seed, |
| | tuber, root, flower |
| Fallopia sachalinensis | Leaf, pulp, fruit, bulb, seed, |
| | tuber, root, flower |
| Flacourtia jangomas | Leaf, pulp, fruit, bulb, seed, |
| | tuber, root, flower |
| Forsythia suspensa | Leaf, pulp, fruit, bulb, seed, |
| | tuber, root, flower |
| Frangula alnus | Leaf, pulp, fruit, bulb, seed, |
| | tuber, root, flower |
| Frangula purshiana | Leaf, pulp, fruit, bulb, seed, |
| | tuber, root, flower |
| Fridericia triplinervia | Leaf, pulp, fruit, bulb, seed, |
| | tuber, root, flower |
| Gaillardia pulchella | Leaf, pulp, fruit, bulb, seed, |
| | tuber, root, flower |
| Galium aparine | Leaf, pulp, fruit, bulb, seed, |
| | tuber, root, flower |
| Garcinia polyantha | Leaf, pulp, fruit, bulb, seed, |
| | tuber, root, flower |
| Gardenia jasminoides | Leaf, pulp, fruit, bulb, seed, |
| | tuber, root, flower |
| Geranium maculatum | Leaf, pulp, fruit, bulb, seed, |
| | tuber, root, flower |
| Gleichenia quadripartita | Leaf, pulp, fruit, bulb, seed, |
| | tuber, root, flower |
| Glycine max | Leaf, pulp, fruit, bulb, seed, |
| | tuber, root, flower |
| Glycyrrhiza uralensis | Leaf, pulp, fruit, bulb, seed, |
| | tuber, root, flower |
| Gnaphalii affinis herba | Leaf, pulp, fruit, bulb, seed, |
| | tuber, root, flower |
| Grangea maderaspatana | Leaf, pulp, fruit, bulb, seed, |
| | tuber, root, flower |
| Gynura procumbens | Leaf, pulp, fruit, bulb, seed, |
| | tuber, root, flower |
| Gynura procumbens | Leaf, pulp, fruit, bulb, seed, |
| | tuber, root, flower |
| Gypsophila perfoliate | Leaf, pulp, fruit, bulb, seed, |
| | tuber, root, flower |
| Habranthus brachyandrus | Leaf, pulp, fruit, bulb, seed, |
| | tuber, root, flower |
| Hedera helix | Leaf, pulp, fruit, bulb, seed, |
| | tuber, root, flower |
| Helichrysum chionosphaerum | Leaf, pulp, fruit, bulb, seed, |
| | tuber, root, flower |
| Helichrysum plicatum | Leaf, pulp, fruit, bulb, seed, |
| | tuber, root, flower |
| Heynea trijuga | Leaf, pulp, fruit, bulb, seed, |
| | tuber, root, flower |
| Hordeum vulgare cv. Seco | Leaf, pulp, fruit, bulb, seed, |
| | tuber, root, flower |
| Hoveniae dulcis semen | Leaf, pulp, fruit, bulb, seed, |
| | tuber, root, flower |
| Hylocomium splendens | Leaf, pulp, fruit, bulb, seed, |
| | tuber, root, flower |
| Hymenoxys lemmonii | Leaf, pulp, fruit, bulb, seed, |
| | tuber, root, flower |
| Hypericum perforatum | Leaf, pulp, fruit, bulb, seed, |
| | tuber, root, flower |
| Hypericum prolificum | Leaf, pulp, fruit, bulb, seed, |
| | tuber, root, flower |
| Hypericum sampsonii | Leaf, pulp, fruit, bulb, seed, |
| | tuber, root, flower |
| Isatdis radix | Leaf, pulp, fruit, bulb, seed, |
| | tuber, root, flower |
| Isatis tinctoria | Leaf, pulp, fruit, bulb, seed, |
| | tuber, root, flower |
| Jacquemontia paniculata | Leaf, pulp, fruit, bulb, seed, |
| | tuber, root, flower |
| Julbernardia globiflora | Leaf, pulp, fruit, bulb, seed, |
| | tuber, root, flower |
| Juniperus drupacea | Leaf, pulp, fruit, bulb, seed, |
| | tuber, root, flower |
| Juniperus formosana | Leaf, pulp, fruit, bulb, seed, |
| | tuber, root, flower |
| Juniperus monosperma | Leaf, pulp, fruit, bulb, seed, |
| | tuber, root, flower |
| Justicia secunda | Leaf, pulp, fruit, bulb, seed, |
| | tuber, root, flower |
| Kniphofia ensifolia | Leaf, pulp, fruit, bulb, seed, |
| | tuber, root, flower |
| Kniphofia foliosa | Leaf, pulp, fruit, bulb, seed, |
| | tuber, root, flower |
| Kniphofia insignis | Leaf, pulp, fruit, bulb, seed, |
| | tuber, root, flower |
| Kniphofia isoetifolia | Leaf, pulp, fruit, bulb, seed, |
| | tuber, root, flower |
| Kniphofia schimperi | Leaf, pulp, fruit, bulb, seed, |
| | tuber, root, flower |
| Lactuca plumieri | Leaf, pulp, fruit, bulb, seed, |
| | tuber, root, flower |
| Lactuca sativa | Leaf, pulp, fruit, bulb, seed, |
| | tuber, root, flower |
| Lanaria lanata | Leaf, pulp, fruit, bulb, seed, |
| | tuber, root, flower |
| Larix kaempferi | Leaf, pulp, fruit, bulb, seed, |
| | tuber, root, flower |
| Larrea tridentata | Leaf, pulp, fruit, bulb, seed, |
| | tuber, root, flower |
| Larrea tridentata | Leaf, pulp, fruit, bulb, seed, |
| | tuber, root, flower |
| Lavandula vera | Leaf, pulp, fruit, bulb, seed, |
| | tuber, root, flower |
| Lepidium meyenii | Leaf, pulp, fruit, bulb, seed, |
| | tuber, root, flower |
| Leptocereus quadricostatus | Leaf, pulp, fruit, bulb, seed, |
| | tuber, root, flower |
| Lespedeza tomentosa | Leaf, pulp, fruit, bulb, seed, |
| | tuber, root, flower |
| Ligularia pleurocaulis | Leaf, pulp, fruit, bulb, seed, |
| | tuber, root, flower |
| Lilii bulbus | Leaf, pulp, fruit, bulb, seed, |
| | tuber, root, flower |
| Lilium brownii f.e. | Leaf, pulp, fruit, bulb, seed, |
| brown var. colchesteri wils | tuber, root, flower |
| Lilium brownii var. viridulum | Leaf, pulp, fruit, bulb, seed, |
| [Syn. Lilium brown | tuber, root, flower |
| Lilium brownii var. | Leaf, pulp, fruit, bulb, seed, |
| viridulum | tuber, root, flower |
| Linum usitatissimum | Leaf, pulp, fruit, bulb, seed, |
| | tuber, root, flower |
| Lobelia berlandieri | Leaf, pulp, fruit, bulb, seed, |
| | tuber, root, flower |
| Loranthus pentandrus | Leaf, pulp, fruit, bulb, seed, |
| | tuber, root, flower |
| Lupinus corymbosus | Leaf, pulp, fruit, bulb, seed, |
| | tuber, root, flower |
| Lycii cortex | Leaf, pulp, fruit, bulb, seed, |
| | tuber, root, flower |
| Maclura tinctoria | Leaf, pulp, fruit, bulb, seed, |
| | tuber, root, flower |
| Macrotyloma uniflorum | Leaf, pulp, fruit, bulb, seed, |
| | tuber, root, flower |
| Magnolia figo | Leaf, pulp, fruit, bulb, seed, |
| | tuber, root, flower |
| Magnolia ovata | Leaf, pulp, fruit, bulb, seed, |
| | tuber, root, flower |
| Malus domestica | Leaf, pulp, fruit, bulb, seed, |
| | tuber, root, flower |
| Mandragora autumnalis | Leaf, pulp, fruit, bulb, seed, |
| | tuber, root, flower |
| Medicago sativa subsp. | Leaf, pulp, fruit, bulb, seed, |
| sativa | tuber, root, flower |
| Melicope triphylla | Leaf, pulp, fruit, bulb, seed, |
| | tuber, root, flower |
| Mentha arvensis | Leaf, pulp, fruit, bulb, seed, |
| | tuber, root, flower |
| Mentha canadensis | Leaf, pulp, fruit, bulb, seed, |
| | tuber, root, flower |
| Menthae herba | Leaf, pulp, fruit, bulb, seed, |
| | tuber, root, flower |
| Metrodorea nigra | Leaf, pulp, fruit, bulb, seed, |
| | tuber, root, flower |
| Miliusa velutina | Leaf, pulp, fruit, bulb, seed, |
| | tuber, root, flower |
| Mirabilis jalapa | Leaf, pulp, fruit, bulb, seed, |
| | tuber, root, flower |
| Mitracarpus scaber | Leaf, pulp, fruit, bulb, seed, |
| | tuber, root, flower |
| Momordica charantia | Leaf, pulp, fruit, bulb, seed, |
| | tuber, root, flower |
| Monactis macbridei | Leaf, pulp, fruit, bulb, seed, |
| | tuber, root, flower |
| Morinda royoc | Leaf, pulp, fruit, bulb, seed, |
| | tuber, root, flower |
| Muehlenbeckia tamnifolia | Leaf, pulp, fruit, bulb, seed, |
| | tuber, root, flower |
| Muntingia calabura | Leaf, pulp, fruit, bulb, seed, |
| | tuber, root, flower |
| Musa acuminata | Leaf, pulp, fruit, bulb, seed, |
| | tuber, root, flower |
| Myrsine africana | Leaf, pulp, fruit, bulb, seed, |
| | tuber, root, flower |
| Narcissus pseudonarcissus | Leaf, pulp, fruit, bulb, seed, |
| | tuber, root, flower |
| Nekemias grossedentata | Leaf, pulp, fruit, bulb, seed, |
| | tuber, root, flower |
| Nelumbo nucifera | Leaf, pulp, fruit, bulb, seed, |
| | tuber, root, flower |
| Neobalanocarpus heimii | Leaf, pulp, fruit, bulb, seed, |
| | tuber, root, flower |
| Neolamarckia cadamba | Leaf, pulp, fruit, bulb, seed, |
| | tuber, root, flower |
| Nepenthes x hookeriana | Leaf, pulp, fruit, bulb, seed, |
| | tuber, root, flower |
| Nepeta granatensis | Leaf, pulp, fruit, bulb, seed, |
| | tuber, root, flower |
| Nepeta parnassica | Leaf, pulp, fruit, bulb, seed, |
| | tuber, root, flower |
| Nicotiana tabacum | Leaf, pulp, fruit, bulb, seed, |
| | tuber, root, flower |
| Nuxia floribunda | Leaf, pulp, fruit, bulb, seed, |
| | tuber, root, flower |
| Nypa fruticans | Leaf, pulp, fruit, bulb, seed, |
| | tuber, root, flower |
| Onobrychis arenaria | Leaf, pulp, fruit, bulb, seed, |
| | tuber, root, flower |
| Oryctes monoceros | Leaf, pulp, fruit, bulb, seed, |
| | tuber, root, flower |
| Oryza sativa | Leaf, pulp, fruit, bulb, seed, |
| | tuber, root, flower |
| Osmanthus armatus | Leaf, pulp, fruit, bulb, seed, |
| | tuber, root, flower |
| Paeonia lactiflora | Leaf, pulp, fruit, bulb, seed, |
| | tuber, root, flower |
| Passiflora sexflora | Leaf, pulp, fruit, bulb, seed, |
| | tuber, root, flower |
| Penstemon centranthifolius | Leaf, pulp, fruit, bulb, seed, |
| | tuber, root, flower |
| Persicaria decipiens | Leaf, pulp, fruit, bulb, seed, |
| | tuber, root, flower |
| Persicaria hydropiper | Leaf, pulp, fruit, bulb, seed, |
| | tuber, root, flower |
| Persicaria maculosa | Leaf, pulp, fruit, bulb, seed, |
| | tuber, root, flower |
| Persicaria perfoliata | Leaf, pulp, fruit, bulb, seed, |
| | tuber, root, flower |
| Persicaria salicifolia | Leaf, pulp, fruit, bulb, seed, |
| | tuber, root, flower |
| Persicaria tinctoria | Leaf, pulp, fruit, bulb, seed, |
| | tuber, root, flower |
| Petunia integrifolia subsp. | Leaf, pulp, fruit, bulb, seed, |
| inflata | tuber, root, flower |
| Phanera guianensis | Leaf, pulp, fruit, bulb, seed, |
| | tuber, root, flower |
| Phaseolus vulgaris | Leaf, pulp, fruit, bulb, seed, |
| | tuber, root, flower |
| Phlogacanthus curviflorus | Leaf, pulp, fruit, bulb, seed, |
| | tuber, root, flower |
| Photinia serratifolia | Leaf, pulp, fruit, bulb, seed, |
| | tuber, root, flower |
| Phyllanthus emblica | Leaf, pulp, fruit, bulb, seed, |
| | tuber, root, flower |
| Physalis alkekengi | Leaf, pulp, fruit, bulb, seed, |
| | tuber, root, flower |
| Picramnia antidesma | Leaf, pulp, fruit, bulb, seed, |
| | tuber, root, flower |
| Picramnia antidesma | Leaf, pulp, fruit, bulb, seed, |
| | tuber, root, flower |
| Picramnia hirsuta | Leaf, pulp, fruit, bulb, seed, |
| | tuber, root, flower |
| Picramnia teapensis | Leaf, pulp, fruit, bulb, seed, |
| | tuber, root, flower |
| Picramnia teapensis Tul. | Leaf, pulp, fruit, bulb, seed, |
| | tuber, root, flower |
| Picrasma quassioides | Leaf, pulp, fruit, bulb, seed, |
| | tuber, root, flower |
| Pinalia stricta | Leaf, pulp, fruit, bulb, seed, |
| | tuber, root, flower |
| Pisum sativum | Leaf, pulp, fruit, bulb, seed, |
| | tuber, root, flower |
| Plantago coronopus | Leaf, pulp, fruit, bulb, seed, |
| | tuber, root, flower |
| Plantago lanceolata | Leaf, pulp, fruit, bulb, seed, |
| | tuber, root, flower |
| Pleione bulbocodioides | Leaf, pulp, fruit, bulb, seed, |
| | tuber, root, flower |
| Pleione yunnanensis | Leaf, pulp, fruit, bulb, seed, |
| | tuber, root, flower |
| Pleurotus ostreatus | Leaf, pulp, fruit, bulb, seed, |
| | tuber, root, flower |
| Ploiarium alternifolium | Leaf, pulp, fruit, bulb, seed, |
| | tuber, root, flower |
| Pluchea odora | Leaf, pulp, fruit, bulb, seed, |
| | tuber, root, flower |
| Plukenetia volubilis | Leaf, pulp, fruit, bulb, seed, |
| | tuber, root, flower |
| Pneumatopteris pennigera | Leaf, pulp, fruit, bulb, seed, |
| | tuber, root, flower |
| Podocarpus spicatus | Leaf, pulp, fruit, bulb, seed, |
| | tuber, root, flower |
| Polygoni cuspidati rhizoma | Leaf, pulp, fruit, bulb, seed, |
| et radix | tuber, root, flower |
| Polygonum cuspidatum | Leaf, pulp, fruit, bulb, seed, |
| | tuber, root, flower |
| Polygonum hypoleucum | Leaf, pulp, fruit, bulb, seed, |
| | tuber, root, flower |
| Polygonum multiflorum | Leaf, pulp, fruit, bulb, seed, |
| | tuber, root, flower |
| Polygonum perfoliatum | Leaf, pulp, fruit, bulb, seed, |
| | tuber, root, flower |
| Pseudolarix amabilis | Leaf, pulp, fruit, bulb, seed, |
| | tuber, root, flower |
| Pseudotsuga menziesii var. | Leaf, pulp, fruit, bulb, seed, |
| menziesii | tuber, root, flower |
| Psidium guajava | Leaf, pulp, fruit, bulb, seed, |
| | tuber, root, flower |
| Psorospermum glaberrimum | Leaf, pulp, fruit, bulb, seed, |
| | tuber, root, flower |
| Psorospermum laurentii | Leaf, pulp, fruit, bulb, seed, |
| | tuber, root, flower |
| Psorospermum tenuifolium | Leaf, pulp, fruit, bulb, seed, |
| | tuber, root, flower |
| Psychotria stachyoides | Leaf, pulp, fruit, bulb, seed, |
| | tuber, root, flower |
| Quercus rubra | Leaf, pulp, fruit, bulb, seed, |
| | tuber, root, flower |
| Radix Boehmeriae | Leaf, pulp, fruit, bulb, seed, |
| | tuber, root, flower |
| Radix et Rhizoma Rhei | Leaf, pulp, fruit, bulb, seed, |
| | tuber, root, flower |
| Radix et Rhizoma Rhei Praeparata | Leaf, pulp, fruit, bulb, seed, |
| | tuber, root, flower |
| Raphanus sativus var. niger | Leaf, pulp, fruit, bulb, seed, |
| | tuber, root, flower |
| Ravenia spectabilis | Leaf, pulp, fruit, bulb, seed, |
| | tuber, root, flower |
| Reynoutria japonica | Leaf, pulp, fruit, bulb, seed, |
| | tuber, root, flower |
| Rhamnus alaternus | Leaf, pulp, fruit, bulb, seed, |
| | tuber, root, flower |
| Rhamnus cathartica | Leaf, pulp, fruit, bulb, seed, |
| | tuber, root, flower |
| Rhamnus davurica | Leaf, pulp, fruit, bulb, seed, |
| | tuber, root, flower |
| Rhamnus erythroxyloides | Leaf, pulp, fruit, bulb, seed, |
| | tuber, root, flower |
| Rhamnus formosana | Leaf, pulp, fruit, bulb, seed, |
| | tuber, root, flower |
| Rhamnus frangula [Syn. | Leaf, pulp, fruit, bulb, seed, |
| Frangula alnus] | tuber, root, flower |
| Rhamnus hirtella | Leaf, pulp, fruit, bulb, seed, |
| | tuber, root, flower |
| Rhamnus kurdica | Leaf, pulp, fruit, bulb, seed, |
| | tuber, root, flower |
| Rhamnus nakaharae | Leaf, pulp, fruit, bulb, seed, |
| | tuber, root, flower |
| Rhamnus nepalensis | Leaf, pulp, fruit, bulb, seed, |
| | tuber, root, flower |
| Rhamnus pallasii | Leaf, pulp, fruit, bulb, seed, |
| | tuber, root, flower |
| Rhamnus prinoides | Leaf, pulp, fruit, bulb, seed, |
| | tuber, root, flower |
| Rhamnus procumbens | Leaf, pulp, fruit, bulb, seed, |
| | tuber, root, flower |
| Rhamnus punctata | Leaf, pulp, fruit, bulb, seed, |
| | tuber, root, flower |
| Rhamnus saxatilis | Leaf, pulp, fruit, bulb, seed, |
| | tuber, root, flower |
| Rhamnus thymifolius | Leaf, pulp, fruit, bulb, seed, |
| | tuber, root, flower |
| Rhamnus triquetra | Leaf, pulp, fruit, bulb, seed, |
| | tuber, root, flower |
| Rhamnus wightii | Leaf, pulp, fruit, bulb, seed, |
| | tuber, root, flower |
| Rheum acuminatum | Leaf, pulp, fruit, bulb, seed, |
| | tuber, root, flower |
| Rheum alexandrae | Leaf, pulp, fruit, bulb, seed, |
| | tuber, root, flower |
| Rheum altaicum | Leaf, pulp, fruit, bulb, seed, |
| | tuber, root, flower |
| Rheum australe | Leaf, pulp, fruit, bulb, seed, |
| | tuber, root, flower |
| Rheum compactum | Leaf, pulp, fruit, bulb, seed, |
| | tuber, root, flower |
| Rheum coreanum | Leaf, pulp, fruit, bulb, seed, |
| | tuber, root, flower |
| Rheum delavayi | Leaf, pulp, fruit, bulb, seed, |
| | tuber, root, flower |
| Rheum emodi [Syn. | Leaf, pulp, fruit, bulb, seed, |
| Rheum australe] | tuber, root, flower |
| Rheum franzenbachii | Leaf, pulp, fruit, bulb, seed, |
| | tuber, root, flower |
| Rheum hotaoense | Leaf, pulp, fruit, bulb, seed, |
| | tuber, root, flower |
| Rheum inopinatum | Leaf, pulp, fruit, bulb, seed, |
| | tuber, root, flower |
| Rheum kialense | Leaf, pulp, fruit, bulb, seed, |
| | tuber, root, flower |
| Rheum likiangense | Leaf, pulp, fruit, bulb, seed, |
| | tuber, root, flower |
| Rheum nanum | Leaf, pulp, fruit, bulb, seed, |
| | tuber, root, flower |
| Rheum nobile | Leaf, pulp, fruit, bulb, seed, |
| | tuber, root, flower |
| Rheum officinale | Leaf, pulp, fruit, bulb, seed, |
| | tuber, root, flower |
| Rheum palmatum | Leaf, pulp, fruit, bulb, seed, |
| | tuber, root, flower |
| Rheum pumilum | Leaf, pulp, fruit, bulb, seed, |
| | tuber, root, flower |
| Rheum racemiferum | Leaf, pulp, fruit, bulb, seed, |
| | tuber, root, flower |
| Rheum reticulatum | Leaf, pulp, fruit, bulb, seed, |
| | tuber, root, flower |
| Rheum rhabarbarum | Leaf, pulp, fruit, bulb, seed, |
| | tuber, root, flower |
| Rheum ribes | Leaf, pulp, fruit, bulb, seed, |
| | tuber, root, flower |
| Rheum spiciforme | Leaf, pulp, fruit, bulb, seed, |
| | tuber, root, flower |
| Rheum spp. | Leaf, pulp, fruit, bulb, seed, |
| | tuber, root, flower |
| Rheum subacaule | Leaf, pulp, fruit, bulb, seed, |
| | tuber, root, flower |
| Rheum sublanceolatum | Leaf, pulp, fruit, bulb, seed, |
| | tuber, root, flower |
| Rheum tanguticum | Leaf, pulp, fruit, bulb, seed, |
| | tuber, root, flower |
| Rheum tataricum | Leaf, pulp, fruit, bulb, seed, |
| | tuber, root, flower |
| Rheum undulatum | Leaf, pulp, fruit, bulb, seed, |
| | tuber, root, flower |
| Rheum uninerve | Leaf, pulp, fruit, bulb, seed, |
| | tuber, root, flower |
| Rheum wittrocki | Leaf, pulp, fruit, bulb, seed, |
| | tuber, root, flower |
| Rheum yunnanense | Leaf, pulp, fruit, bulb, seed, |
| | tuber, root, flower |
| Rhizoma fagopyri cymosi | Leaf, pulp, fruit, bulb, seed, |
| | tuber, root, flower |
| Rhodiola viridula | Leaf, pulp, fruit, bulb, seed, |
| | tuber, root, flower |
| Rhododendron dahuricum | Leaf, pulp, fruit, bulb, seed, |
| | tuber, root, flower |
| Rohdea chinensis | Leaf, pulp, fruit, bulb, seed, |
| | tuber, root, flower |
| Rosa pomifera | Leaf, pulp, fruit, bulb, seed, |
| | tuber, root, flower |
| Rosmarinus officinalis | Leaf, pulp, fruit, bulb, seed, |
| | tuber, root, flower |
| Rumex abyssinicus | Leaf, pulp, fruit, bulb, seed, |
| | tuber, root, flower |
| Rumex acetosa | Leaf, pulp, fruit, bulb, seed, |
| | tuber, root, flower |
| Rumex acetosella | Leaf, pulp, fruit, bulb, seed, |
| | tuber, root, flower |
| Rumex aegyptiacus | Leaf, pulp, fruit, bulb, seed, |
| | tuber, root, flower |
| Rumex alpinus | Leaf, pulp, fruit, bulb, seed, |
| | tuber, root, flower |
| Rumex bequaertii | Leaf, pulp, fruit, bulb, seed, |
| | tuber, root, flower |
| Rumex chalepensis | Leaf, pulp, fruit, bulb, seed, |
| | tuber, root, flower |
| Rumex crispus | Leaf, pulp, fruit, bulb, seed, |
| | tuber, root, flower |
| Rumex cristatus | Leaf, pulp, fruit, bulb, seed, |
| | tuber, root, flower |
| Rumex cyprius | Leaf, pulp, fruit, bulb, seed, |
| | tuber, root, flower |
| Rumex dentatus | Leaf, pulp, fruit, bulb, seed, |
| | tuber, root, flower |
| Rumex japonicus | Leaf, pulp, fruit, bulb, seed, |
| | tuber, root, flower |
| Rumex lanceolatus | Leaf, pulp, fruit, bulb, seed, |
| | tuber, root, flower |
| Rumex limoniastrum | Leaf, pulp, fruit, bulb, seed, |
| | tuber, root, flower |
| Rumex nepalensis | Leaf, pulp, fruit, bulb, seed, |
| | tuber, root, flower |
| Rumex patientia | Leaf, pulp, fruit, bulb, seed, |
| | tuber, root, flower |
| Rumex paulsenianus | Leaf, pulp, fruit, bulb, seed, |
| | tuber, root, flower |
| Rumex pictus | Leaf, pulp, fruit, bulb, seed, |
| | tuber, root, flower |
| Rumex pulcher | Leaf, pulp, fruit, bulb, seed, |
| | tuber, root, flower |
| Rumex ruwenzoriensis | Leaf, pulp, fruit, bulb, seed, |
| | tuber, root, flower |
| Rumex scutatus | Leaf, pulp, fruit, bulb, seed, |
| | tuber, root, flower |
| Rumex simpliciflorus | Leaf, pulp, fruit, bulb, seed, |
| | tuber, root, flower |
| Rumex spp. | Leaf, pulp, fruit, bulb, seed, |
| | tuber, root, flower |
| Rumex tuberosus | Leaf, pulp, fruit, bulb, seed, |
| | tuber, root, flower |
| Rumex usambarensis | Leaf, pulp, fruit, bulb, seed, |
| | tuber, root, flower |
| Rumex vesicarius | Leaf, pulp, fruit, bulb, seed, |
| | tuber, root, flower |
| Salvia aurea | Leaf, pulp, fruit, bulb, seed, |
| | tuber, root, flower |
| Salvia greggii | Leaf, pulp, fruit, bulb, seed, |
| | tuber, root, flower |
| Sargentodoxa cuneata | Leaf, pulp, fruit, bulb, seed, |
| | tuber, root, flower |
| Sargentodoxae caulis | Leaf, pulp, fruit, bulb, seed, |
| | tuber, root, flower |
| Saururus chinensis | Leaf, pulp, fruit, bulb, seed, |
| | tuber, root, flower |
| Schistostephium artemisiifolium | Leaf, pulp, fruit, bulb, seed, |
| | tuber, root, flower |
| Scilla socialis | Leaf, pulp, fruit, bulb, seed, |
| | tuber, root, flower |
| Secale cereale cv. | Leaf, pulp, fruit, bulb, seed, |
| Aroostook | tuber, root, flower |
| Selaginella delicatula | Leaf, pulp, fruit, bulb, seed, |
| | tuber, root, flower |
| Selaginella labordei | Leaf, pulp, fruit, bulb, seed, |
| | tuber, root, flower |
| Semen pharbitidis | Leaf, pulp, fruit, bulb, seed, |
| | tuber, root, flower |
| Senecio behnii | Leaf, pulp, fruit, bulb, seed, |
| | tuber, root, flower |
| Senecio bonariensis | Leaf, pulp, fruit, bulb, seed, |
| | tuber, root, flower |
| Senecio congestus | Leaf, pulp, fruit, bulb, seed, |
| | tuber, root, flower |
| Senecio praecox | Leaf, pulp, fruit, bulb, seed, |
| | tuber, root, flower |
| Senecio subdentatus | Leaf, pulp, fruit, bulb, seed, |
| | tuber, root, flower |
| Senna alata | Leaf, pulp, fruit, bulb, seed, |
| | tuber, root, flower |
| Senna alexandrina | Leaf, pulp, fruit, bulb, seed, |
| | tuber, root, flower |
| Senna angustisiliqua | Leaf, pulp, fruit, bulb, seed, |
| | tuber, root, flower |
| Senna artemisioides | Leaf, pulp, fruit, bulb, seed, |
| | tuber, root, flower |
| Senna auriculata | Leaf, pulp, fruit, bulb, seed, |
| | tuber, root, flower |
| Senna corymbosa | Leaf, pulp, fruit, bulb, seed, |
| | tuber, root, flower |
| Senna didymobotrya | Leaf, pulp, fruit, bulb, seed, |
| | tuber, root, flower |
| Senna didymobotrya | Leaf, pulp, fruit, bulb, seed, |
| | tuber, root, flower |
| Senna lindheimeriana | Leaf, pulp, fruit, bulb, seed, |
| | tuber, root, flower |
| Senna macranthera | Leaf, pulp, fruit, bulb, seed, |
| | tuber, root, flower |
| Senna obtusifolia | Leaf, pulp, fruit, bulb, seed, |
| | tuber, root, flower |
| Senna occidentalis | Leaf, pulp, fruit, bulb, seed, |
| | tuber, root, flower |
| Senna petersiana | Leaf, pulp, fruit, bulb, seed, |
| | tuber, root, flower |
| Senna podocarpa | Leaf, pulp, fruit, bulb, seed, |
| | tuber, root, flower |
| Senna reticulata | Leaf, pulp, fruit, bulb, seed, |
| | tuber, root, flower |
| Senna septemtrionalis | Leaf, pulp, fruit, bulb, seed, |
| | tuber, root, flower |
| Senna siamea | Leaf, pulp, fruit, bulb, seed, |
| | tuber, root, flower |
| Senna sophera | Leaf, pulp, fruit, bulb, seed, |
| | tuber, root, flower |
| Senna spectabilis | Leaf, pulp, fruit, bulb, seed, |
| | tuber, root, flower |
| Senna tora | Leaf, pulp, fruit, bulb, seed, |
| | tuber, root, flower |
| Sennae folium | Leaf, pulp, fruit, bulb, seed, |
| | tuber, root, flower |
| Silybum marianum | Leaf, pulp, fruit, bulb, seed, |
| | tuber, root, flower |
| Simaba orinocensis | Leaf, pulp, fruit, bulb, seed, |
| | tuber, root, flower |
| Smythea bombaiensis | Leaf, pulp, fruit, bulb, seed, |
| | tuber, root, flower |
| Solanum americanum | Leaf, pulp, fruit, bulb, seed, |
| | tuber, root, flower |
| Solanum cleistogamum | Leaf, pulp, fruit, bulb, seed, |
| | tuber, root, flower |
| Solanum lycopersicum | Leaf, pulp, fruit, bulb, seed, |
| | tuber, root, flower |
| Solanum melongena | Leaf, pulp, fruit, bulb, seed, |
| | tuber, root, flower |
| Sophorae tonkinensis | Leaf, pulp, fruit, bulb, seed, |
| radix et rhizome | tuber, root, flower |
| Soymida febrifuga | Leaf, pulp, fruit, bulb, seed, |
| | tuber, root, flower |
| Spatholobus suberectus | Leaf, pulp, fruit, bulb, seed, |
| | tuber, root, flower |
| Sphaerella rosigena | Leaf, pulp, fruit, bulb, seed, |
| | tuber, root, flower |
| Spinacia oleracea | Leaf, pulp, fruit, bulb, seed, |
| | tuber, root, flower |
| Stephania dinklagei | Leaf, pulp, fruit, bulb, seed, |
| | tuber, root, flower |
| Stevia berlandieri | Leaf, pulp, fruit, bulb, seed, |
| | tuber, root, flower |
| Stevia ovata | Leaf, pulp, fruit, bulb, seed, |
| | tuber, root, flower |
| Syzygium australe | Leaf, pulp, fruit, bulb, seed, |
| | tuber, root, flower |
| Tagetes filifolia | Leaf, pulp, fruit, bulb, seed, |
| | tuber, root, flower |
| Tanacetum coccineum | Leaf, pulp, fruit, bulb, seed, |
| | tuber, root, flower |
| Taraxacum kok-saghyz | Leaf, pulp, fruit, bulb, seed, |
| | tuber, root, flower |
| Taraxacum platycarpum subsp. | Leaf, pulp, fruit, bulb, seed, |
| hondoense | tuber, root, flower |
| Taxus brevifolia | Leaf, pulp, fruit, bulb, seed, |
| | tuber, root, flower |
| Tetradium ruticarpum | Leaf, pulp, fruit, bulb, seed, |
| | tuber, root, flower |
| Teucrium yemense | Leaf, pulp, fruit, bulb, seed, |
| | tuber, root, flower |
| Theobroma cacao | Leaf, pulp, fruit, bulb, seed, |
| | tuber, root, flower |
| Thymelaea hirsuta | Leaf, pulp, fruit, bulb, seed, |
| | tuber, root, flower |
| Tribuli fructus | Leaf, pulp, fruit, bulb, seed, |
| | tuber, root, flower |
| Tribulus terrestris | Leaf, pulp, fruit, bulb, seed, |
| | tuber, root, flower |
| Trifolium pratense cv. | Leaf, pulp, fruit, bulb, seed, |
| Redman | tuber, root, flower |
| Tripterygium wilfordii | Leaf, pulp, fruit, bulb, seed, |
| | tuber, root, flower |
| Tripterygium wilfordii | Leaf, pulp, fruit, bulb, seed, |
| | tuber, root, flower |
| Tupistra chinensis | Leaf, pulp, fruit, bulb, seed, |
| | tuber, root, flower |
| Uncaria tomentosa | Leaf, pulp, fruit, bulb, seed, |
| | tuber, root, flower |
| Uvaria afzelii | Leaf, pulp, fruit, bulb, seed, |
| | tuber, root, flower |
| Vatairea heteroptera | Leaf, pulp, fruit, bulb, seed, |
| | tuber, root, flower |
| Ventilago calyculata | Leaf, pulp, fruit, bulb, seed, |
| | tuber, root, flower |
| Ventilago denticulata | Leaf, pulp, fruit, bulb, seed, |
| | tuber, root, flower |
| Ventilago leiocarpa | Leaf, pulp, fruit, bulb, seed, |
| | tuber, root, flower |
| Ventilago madraspatana | Leaf, pulp, fruit, bulb, seed, |
| | tuber, root, flower |
| Verbascum thapsiforme | Leaf, pulp, fruit, bulb, seed, |
| | tuber, root, flower |
| Veronica polita | Leaf, pulp, fruit, bulb, seed, |
| | tuber, root, flower |
| Vismia guianensis | Leaf, pulp, fruit, bulb, seed, |
| | tuber, root, flower |
| Vismia laurentii | Leaf, pulp, fruit, bulb, seed, |
| | tuber, root, flower |
| Vismia orientalis | Leaf, pulp, fruit, bulb, seed, |
| | tuber, root, flower |
| Vitis tilifolia | Leaf, pulp, fruit, bulb, seed, |
| | tuber, root, flower |
| Vitis vinifera | Leaf, pulp, fruit, bulb, seed, |
| | tuber, root, flower |
| Wedelia trilobata | Leaf, pulp, fruit, bulb, seed, |
| | tuber, root, flower |
| Wilbrandia ebracteata | Leaf, pulp, fruit, bulb, seed, |
| | tuber, root, flower |
| Xanthii fructus | Leaf, pulp, fruit, bulb, seed, |
| | tuber, root, flower |
| Xanthorrhoea australis | Leaf, pulp, fruit, bulb, seed, |
| | tuber, root, flower |
| Xerochrysum viscosum | Leaf, pulp, fruit, bulb, seed, |
| | tuber, root, flower |
| Zea mays | Leaf, pulp, fruit, bulb, seed, |
| | tuber, root, flower |

The amounts of guibourtinidol, or other compounds of interest such as those described herein, present in certain extracts (% of extract, w/w) will be determined. Quantification of the compounds will be performed by normalizing the results by the weight of the extracts. The results of these analyses will be determined.

### Example 3

An oral composition is provided on a daily basis, the oral composition utilizing a caplet formulation comprising guibourtinidol from an extract of Cassia abbreviate and a carrier.

Directions: As an oral composition, 1 capsule is orally administered with an 8 oz. glass of water once daily.

### Example 4

In this example, the plant fractions were isolated and tested to confirm antagonism or inhibition activity.

Dionex™ Accelerated Solvent Extractor™ (“ASE”) 350 was used for the extraction of plant material. One 25 mL stainless steel ASE extraction cell was filled with 5 g of lyophilized, milled plant tissues. The pre-set temperature was 60° C., and for each cell, the static extraction was run for a total of five extraction cycles: 2×95% ethanol (AX), 1×1:1 95% ethanol:ethyl acetate (BX), 1× ethyl acetate (CX), and 1× hexane (DX). Three mL aliquots from each extract AX, BX, CX, and DX were taken and dried using a vacuum concentrator instrument (HT12 3i series, SP Scientific). One additional 3 mL aliquot of the AX extract was treated with 0.5 g poly(vinylpolypyrrolidone), or PVP, resin to create an additional sample (AP). The resin was filtered out using centrifugal filter units (PVDF, 0.45 μm), and a vacuum concentrator dried the remaining supernatant. To generate X0-X6 fractions, extracts from AX, BX, CX, and DX were combined and absorbed onto celite and placed in a solid loading cartridge for C18 spherical flash chromatography fractionation on the Combi Flash (Teledyne). Fractionation was conducted with a mobile phase consisting of water+0.1% formic acid (A) and CH₃CN+0.1% formic acid (B) at a flow rate of 18 mL/min. Two-minute fractions were collected for each isocratic step with increasing polarity (10, 20, 40, 60, 80, 100% B represent X0, X1, X2, X3, X4, X5 respectively) and additional 4 minutes of 100% B collected as an X6 fraction.

Samples were assayed for agonist, positive allosteric modulators (PAM), & antagonist/negative allosteric modulators (NAM) activity for mGluR2 and mGluR3. The primary assays for mGluR2 and mGluR3 activity were performed with the CAMP HitHunter® assay designed and supplied by DiscoverX, with the CHO-K1 GRM2 Gi cell line and CHO-K1 GRM3 Gi cell line for the mGluR2 and mGluR3 assays, respectively. The assay protocol was adjusted to assess differences in the directionality of activity, and the same assay protocol was used for both the mGluR2 and mGluR3 assay. For agonist activity, the assay was run without any internal agonist added, and the effect on receptor activity was measured. For PAM activity, the assay was performed with the addition of a known control agonist, LY354740, at a concentration of 10 nM. Finally, to determine antagonist/NAM activity, the assay was performed with the addition of the same known control agonist at a concentration of 50 nM. Assay screening is unable to differentiate between antagonist and NAM activity as both require the presence of added agonists. Activity on the receptor of interest was confirmed by analysis of phosphorylation of ERK (pERK), a downstream component of the signaling cascade, through western blot. Assay conditions used the same differentiating strategy as above, the addition of different concentrations of control agonist, for samples that had shown the respective activity in the primary assay. Agonist and PAM activity increased pERK, while antagonist/NAM activity decreased pERK.

To address any potential viability issues on assay performance and future toxicity concerns in vivo testing, samples that showed mGluR2 and mGluR3 activity were also measured for cellular metabolic health, viability, and live/dead analysis, a measure of cell death. Viability was measured by the CyQUANT™ XTT assay kit, provided by ThermoFisher, while live/dead assay was measured by the HCS Live/Dead Green kit, provided by Invitrogen.

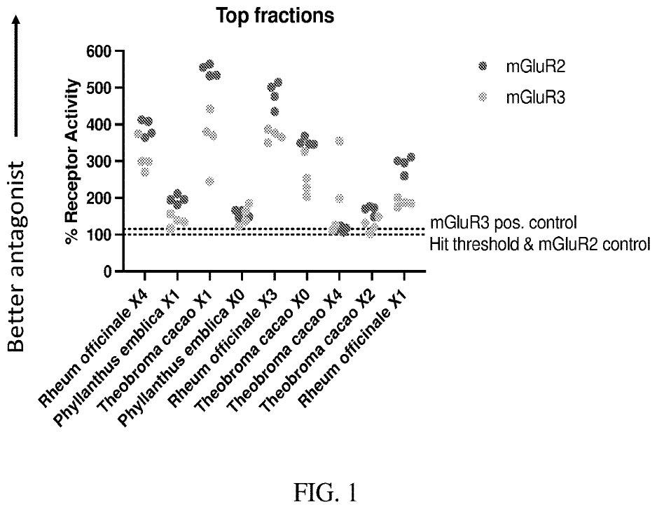

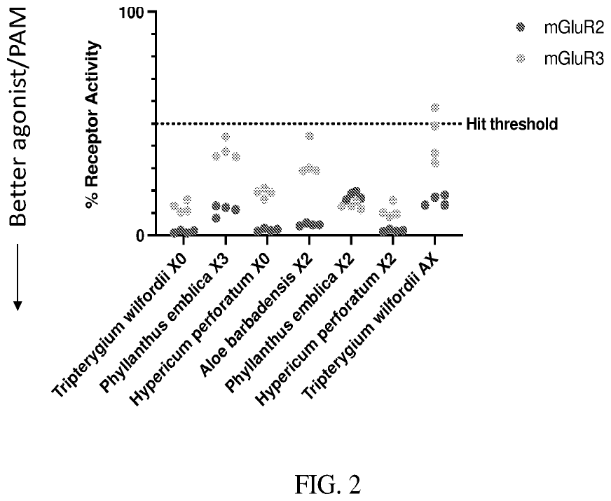

The results of the study are described in Tables 2-6 and FIGS. 1-2.

TABLE 2

| | | | | Bioactivity |
|---|---|---|---|---|
| | Plant | Fraction ID | Modulation type | (in vitro) |
| 1 | Rheum officinale | F_X4 | Antagonist | Yes |
| 2 | Phyllanthus emblica | F_X1 | Antagonist | Yes |
| 3 | Theobroma cacao | F_X1 | Antagonist | Yes |
| 4 | Phyllanthus emblica | F_X0 | Antagonist | Yes |
| 5 | Rheum officinale | F_X3 | Antagonist | Yes |
| 6 | Theobroma cacao | F_X0 | Antagonist | Yes |
| 7 | Theobroma cacao | F_X4 | Antagonist | Yes |
| 8 | Theobroma cacao | F_X2 | Antagonist | Yes |
| 9 | Rheum officinale | F_X1 | Antagonist | Yes |

TABLE 3

| | | Fraction | Modulation | Bioactivity |
|---|---|---|---|---|
| | Plant | ID | type | (in vitro) |
| 1 | Tripterygium wilfordii | F_X0 | Agonist | Yes |
| 2 | Phyllanthus emblica | F_X3 | PAM | Yes |
| 3 | Hypericum perforatum | F_X0 | Agonist | Yes |
| 4 | Aloe barbadensis | F_X2 | Agonist | Yes |
| 5 | Phyllanthus emblica | F_X2 | PAM* | Yes |
| 6 | Hypericum perforatum | F_X2 | Agonist | Yes |
| 7 | Tripterygium wilfordii | F_AX | Agonist | Yes |

*PAM—Positive Allosteric Modulator

TABLE 4

| | | Fraction | Modulation | Bioactivity |
|---|---|---|---|---|
| | Plant | ID | Type | (in vitro) |
| 1 | Rheum officinale | F_DX | Antagonist | Yes |
| 2 | Theobroma cacao | F_X0 | Antagonist | Yes |
| 3 | Hordeum vulgare | F_DX | Antagonist | Yes |
| | cv. Seco | | | |
| 4 | Chaenomeles sinensis | F_CX | Antagonist | Yes |
| 5 | Rheum officinale | F_AP | Antagonist | Yes |
| 6 | Nepenthes × hookeriana | F_X4 | Antagonist | Yes |
| 7 | Musa acuminata | F_BX | Antagonist | Yes |
| 8 | Pseudotsuga menziesii | F_X4 | Antagonist | Yes |
| | var. menziesii | | | |
| 9 | Chaenomeles sinensis | F_BX | Antagonist | Yes |
| 10 | Chaenomeles sinensis | F_DX | Antagonist | Yes |
| 11 | Penstemon centranthifolius | F_X5 | Antagonist | Yes |
| 12 | Pisum sativum | F_X1 | Antagonist | Yes |
| 13 | Aloe africana | F_DX | Antagonist | Yes |
| 14 | Aloe africana | F_BX | Antagonist | Yes |
| 15 | Pisum sativum | F_AX | Antagonist | Yes |
| 16 | Hordeum vulgare cv. Seco | F_X1 | Antagonist | Yes |
| 17 | Penstemon centranthifolius | F_X4 | Antagonist | Yes |
| 18 | Glycine max | F_X6 | Antagonist | Yes |
| 19 | Pseudotsuga menziesii | F_AX | Antagonist | Yes |
| | var. menziesii | | | |
| 20 | Aloe africana | F_CX | Antagonist | Yes |
| 21 | Avena sativa | F_BX | Antagonist | Yes |
| 22 | Glycine max | F_AP | Antagonist | Yes |
| 23 | Musa acuminata | F_X0 | Antagonist | Yes |
| 24 | Malus domestica | F_X2 | Antagonist | Yes |
| 25 | Gardenia jasminoides | F_CX | Antagonist | Yes |
| 26 | Glycine max | F_AX | Antagonist | Yes |
| 27 | Rheum officinale | F_X1 | Antagonist | Yes |
| 28 | Pisum sativum | F_DX | Antagonist | Yes |
| 29 | Malus domestica | F_X3 | Antagonist | Yes |
| 30 | Aloe ferox | F_X0 | Antagonist | Yes |
| 31 | Oryza sativa | F_AP | Antagonist | Yes |
| 32 | Pseudotsuga menziesii | F_CX | Antagonist | Yes |
| | var. menziesii | | | |
| 33 | Coffea arabica | F_X0 | Antagonist | Yes |
| 34 | Glycine max | F_X2 | Antagonist | Yes |
| 35 | Pisum sativum | F_CX | Antagonist | Yes |
| 36 | Carthamus tinctorius | F_AP | Antagonist | Yes |
| 37 | Penstemon centranthifolius | F_X6 | Antagonist | Yes |
| 38 | Glycine max | F_X1 | Antagonist | Yes |
| 39 | Hordeum vulgare | F_AP | Antagonist | Yes |
| | cv. Seco | | | |
| 40 | Hordeum vulgare | F_X0 | Antagonist | Yes |
| | cv. Seco | | | |
| 41 | Macrotyloma uniflorum | F_AX | Antagonist | Yes |
| 42 | Solanum melongena | F_X4 | Antagonist | Yes |
| 43 | Rheum officinale | F_X4 | Antagonist | Yes |
| 44 | Aloe vera | F_DX | Antagonist | Yes |
| 45 | Citrus unshiu | F_CX | Antagonist | Yes |
| 46 | Hypericum perforatum | F_CX | Antagonist | Yes |
| 47 | Malus domestica | F_X1 | Antagonist | Yes |
| 48 | Curcuma longa | F_CX | Antagonist | Yes |
| 49 | Macrotyloma uniflorum | F_BX | Antagonist | Yes |
| 50 | Theobroma cacao | F_X1 | Antagonist | Yes |
| 51 | Oryza sativa | F_DX | Antagonist | Yes |
| 52 | Nypa fruticans | F_BX | Antagonist | Yes |
| 53 | Hordeum vulgare | F_X2 | Antagonist | Yes |
| | cv. Seco | | | |
| 54 | Avena sativa | F_DX | Antagonist | Yes |
| 55 | Penstemon centranthifolius | F_DX | Antagonist | Yes |
| 56 | Secale cereale | F_X2 | Antagonist | Yes |
| | cv. Aroostook | | | |
| 57 | Glycine max | F_DX | Antagonist | Yes |
| 58 | Persicaria tinctoria | F_X4 | Antagonist | Yes |
| 59 | Nypa fruticans | F_AP | Antagonist | Yes |
| 60 | Pisum sativum | F_AP | Antagonist | Yes |
| 61 | Glycine max | F_X5 | Antagonist | Yes |
| 62 | Nypa fruticans | F_AX | Antagonist | Yes |
| 63 | Hordeum vulgare | F_CX | Antagonist | Yes |
| | cv. Seco | | | |
| 64 | Pisum sativum | F_BX | Antagonist | Yes |
| 65 | Crocus chrysanthus | F_CX | Antagonist | Yes |
| 66 | Oryza sativa | F_CX | Antagonist | Yes |
| 67 | Secale cereale | F_X1 | Antagonist | Yes |
| | cv. Aroostook | | | |
| 68 | Glycine max | F_BX | Antagonist | Yes |
| 69 | Pseudotsuga menziesii | F_DX | Antagonist | Yes |
| | var. menziesii | | | |
| 70 | Oryza sativa | F_X2 | Antagonist | Yes |
| 71 | Pisum sativum | F_X2 | Antagonist | Yes |
| 72 | Penstemon centranthifolius | F_CX | Antagonist | Yes |
| 73 | Hordeum vulgare | F_BX | Antagonist | Yes |
| | cv. Seco | | | |
| 74 | Uncaria tomentosa | F_X1 | Antagonist | Yes |
| 75 | Auricularia auricula-judae | F_AP | Antagonist | Yes |
| 76 | Penstemon centranthifolius | F_AP | Antagonist | Yes |
| 77 | Nypa fruticans | F_X1 | Antagonist | Yes |
| 78 | Nepenthes × hookeriana | F_AX | Antagonist | Yes |
| 79 | Citrus clementina | F_DX | Antagonist | Yes |
| 80 | Musa acuminata | F_DX | Antagonist | Yes |
| 81 | Oryza sativa | F_AX | Antagonist | Yes |
| 82 | Penstemon centranthifolius | F_X3 | Antagonist | Yes |
| 83 | Secale cereale | F_CX | Antagonist | Yes |
| | cv. Aroostook | | | |
| 84 | Gardenia jasminoides | F_BX | Antagonist | Yes |
| 85 | Pseudotsuga menziesii | F_BX | Antagonist | Yes |
| | var. menziesii | | | |
| 86 | Rosmarinus officinalis | F_X0 | Antagonist | Yes |
| 87 | Pseudotsuga menziesii | F_AP | Antagonist | Yes |
| | var. menziesii | | | |
| 88 | Cannabis sativa | F_DX | Antagonist | Yes |
| 89 | Solanum lycopersicum | F_CX | Antagonist | Yes |
| 90 | Cannabis sativa | F_CX | Antagonist | Yes |
| 91 | Nepenthes × hookeriana | F_AP | Antagonist | Yes |
| 92 | Glycine max | F_X3 | Antagonist | Yes |
| 93 | Aloe africana | F_AX | Antagonist | Yes |
| 94 | Persicaria tinctoria | F_X3 | Antagonist | Yes |
| 95 | Citrus sinensis | F_BX | Antagonist | Yes |
| 96 | Auricularia auricula-judae | F_X2 | Antagonist | Yes |
| 97 | Penstemon centranthifolius | F_X1 | Antagonist | Yes |
| 98 | Trifolium pratense | F_CX | Antagonist | Yes |
| | cv. Redman | | | |
| 99 | Hordeum vulgare | F_AX | Antagonist | Yes |
| | cv. Seco | | | |
| 100 | Nepenthes × hookeriana | F_BX | Antagonist | Yes |
| 101 | Oryza sativa | F_BX | Antagonist | Yes |
| 102 | Cycas revoluta | F_BX | Antagonist | Yes |
| 103 | Malus domestica | F_X0 | Antagonist | Yes |
| 104 | Caulerpa taxifolia | F_DX | Antagonist | Yes |
| 105 | Anacardium occidentale | F_CX | Antagonist | Yes |
| 106 | Secale cereale | F_BX | Antagonist | Yes |
| | cv. Aroostook | | | |
| 107 | Crocus chrysanthus | F_BX | Antagonist | Yes |
| 108 | Cycas revoluta | F_AX | Antagonist | Yes |
| 109 | Crocus chrysanthus | F_X1 | Antagonist | Yes |
| 110 | Nepenthes × hookeriana | F_X2 | Antagonist | Yes |
| 111 | Glycine max | F_CX | Antagonist | Yes |
| 112 | Aloe africana | F_AP | Antagonist | Yes |
| 113 | Cannabis sativa | F_X1 | Antagonist | Yes |
| 114 | Cycas revoluta | F_AP | Antagonist | Yes |
| 115 | Momordica charantia | F_DX | Antagonist | Yes |
| 116 | Brassica carinata | F_DX | Antagonist | Yes |
| 117 | Malus domestica | F_X4 | Antagonist | Yes |
| 118 | Malus domestica | F_AX | Antagonist | Yes |
| 119 | Persicaria tinctoria | F_CX | Antagonist | Yes |
| 120 | Gaillardia pulchella | F_AP | Antagonist | Yes |
| 121 | Macrotyloma uniflorum | F_AP | Antagonist | Yes |
| 122 | Citrus unshiu | F_DX | Antagonist | Yes |
| 123 | Oryza sativa | F_X3 | Antagonist | Yes |
| 124 | Trifolium pratense | F_X1 | Antagonist | Yes |
| | cv. Redman | | | |
| 125 | Nelumbo nucifera | F_DX | Antagonist | Yes |
| 126 | Raphanus sativus | F_BX | Antagonist | Yes |
| | var. niger | | | |
| 127 | Caulerpa taxifolia | F_AP | Antagonist | Yes |
| 128 | Auricularia auricula-judae | F_X1 | Antagonist | Yes |
| 129 | Cycas revoluta | F_CX | Antagonist | Yes |
| 130 | Trifolium pratense | F_DX | Antagonist | Yes |
| | cv. Redman | | | |
| 131 | Secale cereale | F_AX | Antagonist | Yes |
| | cv. Aroostook | | | |
| 132 | Macrotyloma uniflorum | F_CX | Antagonist | Yes |
| 133 | Penstemon centranthifolius | F_X0 | Antagonist | Yes |
| 134 | Penstemon centranthifolius | F_BX | Antagonist | Yes |
| 135 | Auricularia auricula-judae | F_CX | Antagonist | Yes |
| 136 | Brassica carinata | F_BX | Antagonist | Yes |
| 137 | Trifolium pratense | F_BX | Antagonist | Yes |
| | cv. Redman | | | |
| 138 | Chaenomeles sinensis | F_AX | Antagonist | Yes |
| 139 | Glycine max | F_X4 | Antagonist | Yes |
| 140 | Cannabis sativa | F_X2 | Antagonist | Yes |
| 141 | Uncaria tomentosa | F_DX | Antagonist | Yes |
| 142 | Hypericum perforatum | F_DX | Antagonist | Yes |
| 143 | Theobroma cacao | F_X2 | Antagonist | Yes |
| 144 | Brassica carinata | F_X1 | Antagonist | Yes |
| 145 | Penstemon centranthifolius | F_AX | Antagonist | Yes |
| 146 | Brassica carinata | F_CX | Antagonist | Yes |
| 147 | Rosmarinus officinalis | F_DX | Antagonist | Yes |
| 148 | Lepidium meyenii | F_DX | Antagonist | Yes |
| 149 | Gaillardia pulchella | F_DX | Antagonist | Yes |
| 150 | Oryza sativa | F_X1 | Antagonist | Yes |
| 151 | Pseudotsuga menziesii | F_X0 | Antagonist | Yes |
| | var. menziesii | | | |
| 152 | Solanum melongena | F_AP | Antagonist | Yes |
| 153 | Crocus chrysanthus | F_X0 | Antagonist | Yes |
| 154 | Crocus chrysanthus | F_X2 | Antagonist | Yes |
| 155 | Medicago sativa | F_X1 | Antagonist | Yes |
| | subsp. sativa | | | |
| 156 | Crocus chrysanthus | F_DX | Antagonist | Yes |
| 157 | Aloe vera | F_CX | Antagonist | Yes |
| 158 | Gaillardia pulchella | F_CX | Antagonist | Yes |
| 159 | Cannabis sativa | F_X3 | Antagonist | Yes |
| 160 | Allium macrostemon | F_AP | Antagonist | Yes |
| 161 | Zea mays | F_DX | Antagonist | Yes |
| 162 | Citrus sinensis | F_DX | Antagonist | Yes |
| 163 | Musa acuminata | F_CX | Antagonist | Yes |
| 164 | Nicotiana tabacum | F_CX | Antagonist | Yes |
| 165 | Solanum lycopersicum | F_DX | Antagonist | Yes |
| 166 | Agaricus subrufescens | F_AX | Antagonist | Yes |
| 167 | Citrus sinensis | F_CX | Antagonist | Yes |
| 168 | Avena sativa | F_CX | Antagonist | Yes |
| 169 | Caulerpa taxifolia | F_BX | Antagonist | Yes |
| 170 | Macrotyloma uniflorum | F_X0 | Antagonist | Yes |
| 171 | Solanum lycopersicum | F_BX | Antagonist | Yes |
| 172 | Allium macrostemon | F_BX | Antagonist | Yes |
| 173 | Cycas revoluta | F_DX | Antagonist | Yes |
| 174 | Secale cereale | F_DX | Antagonist | Yes |
| | cv. Aroostook | | | |
| 175 | Linum usitatissimum | F_CX | Antagonist | Yes |
| 176 | Paeonia lactiflora | F_BX | Antagonist | Yes |
| 177 | Malus domestica | F_BX | Antagonist | Yes |
| 178 | Auricularia auricula-judae | F_DX | Antagonist | Yes |
| 179 | Raphanus sativus | F_DX | Antagonist | Yes |
| | var. niger | | | |
| 180 | Uncaria tomentosa | F_BX | Antagonist | Yes |
| 181 | Citrus sinensis | F_X1 | Antagonist | Yes |
| 182 | Brassica carinata | F_AP | Antagonist | Yes |
| 183 | Crocus chrysanthus | F_AP | Antagonist | Yes |
| 184 | Auricularia auricula-judae | F_BX | Antagonist | Yes |
| 185 | Capsicum anmium | F_BX | Antagonist | Yes |
| 186 | Persicaria tinctoria | F_DX | Antagonist | Yes |
| 187 | Larrea tridentata | F_DX | Antagonist | Yes |
| 188 | Persicaria tinctoria | F_BX | Antagonist | Yes |
| 189 | Mirabilis jalapa | F_AX | Antagonist | Yes |
| 190 | Capsicum annuum | F_X0 | Antagonist | Yes |
| 191 | Paeonia lactiflora | F_DX | Antagonist | Yes |
| 192 | Aloe vera | F_AP | Antagonist | Yes |
| 193 | Nepenthes × hookeriana | F_CX | Antagonist | Yes |
| 194 | Lepidium meyenii | F_CX | Antagonist | Yes |
| 195 | Linum usitatissimum | F_BX | Antagonist | Yes |
| 196 | Secale cereale | F_AP | Antagonist | Yes |
| | cv. Aroostook | | | |
| 197 | Caulerpa taxifolia | F_AX | Antagonist | Yes |
| 198 | Catharanthus roseus | F_AP | Antagonist | Yes |
| 199 | Cannabis sativa | F_AP | Antagonist | Yes |
| 200 | Hordeum vulgare | F_X3 | Antagonist | Yes |
| | cv. Seco | | | |
| 201 | Theobroma cacao | F_DX | Antagonist | Yes |
| 202 | Crocus chrysanthus | F_AX | Antagonist | Yes |
| 203 | Nelumbo nucifera | F_CX | Antagonist | Yes |
| 204 | Agaricus subrufescens | F_AP | Antagonist | Yes |
| 205 | Uncaria tomentosa | F_CX | Antagonist | Yes |
| 206 | Catharanthus roseus | F_CX | Antagonist | Yes |
| 207 | Theobroma cacao | F_AX | Antagonist | Yes |
| 208 | Auricularia auricula-judae | F_AX | Antagonist | Yes |
| 209 | Aloe vera | F_BX | Antagonist | Yes |
| 210 | Malus domestica | F_X6 | Antagonist | Yes |
| 211 | Malus domestica | F_AP | Antagonist | Yes |

TABLE 5

| | | Fraction | Modulation | Bioactivity |
|---|---|---|---|---|
| | Plant | ID | Type | (in vitro) |
| 1 | Momordica charantia | F_X3 | Agonist | Yes |
| 2 | Momordica charantia | F_X4 | Agonist | Yes |
| 3 | Momordica charantia | F_X5 | Agonist | Yes |
| 4 | Aloe vera | F_X0 | Agonist | Yes |
| 5 | Aloe vera | F_X1 | Agonist | Yes |
| 6 | Aloe vera | F_X2 | Agonist | Yes |
| 7 | Aloe vera | F_X3 | Agonist | Yes |
| 8 | Aloe vera | F_X4 | Agonist | Yes |
| 9 | Aloe vera | F_X5 | Agonist | Yes |
| 10 | Silybum marianum | F_X3 | Agonist | Yes |
| 11 | Silybum marianum | F_X4 | Agonist | Yes |
| 12 | Silybum marianum | F_X5 | Agonist | Yes |
| 13 | Aloe ferox | F_AX | Agonist | Yes |
| 14 | Aloe ferox | F_X1 | Agonist | Yes |
| 15 | Aloe ferox | F_X2 | Agonist | Yes |
| 16 | Aloe ferox | F_X3 | Agonist | Yes |
| 17 | Aloe ferox | F_X4 | Agonist | Yes |
| 18 | Rheum officinale | F_X2 | Agonist | Yes |
| 19 | Rheum officinale | F_X3 | Agonist | Yes |
| 20 | Chaenomeles sinensis | F_X2 | Agonist | Yes |
| 21 | Chaenomeles sinensis | F_X3 | Agonist | Yes |
| 22 | Citrus clementina | F_AP | Agonist | Yes |
| 23 | Citrus clementina | F_AX | Agonist | Yes |
| 24 | Citrus clementina | F_X3 | Agonist | Yes |
| 25 | Citrus clementina | F_X4 | Agonist | Yes |
| 26 | Citrus clementina | F_X5 | Agonist | Yes |
| 27 | Citrus clementina | F_X6 | Agonist | Yes |
| 28 | Hordeum vulgare cv. Seco | F_X4 | Agonist | Yes |
| 29 | Hordeum vulgare cv. Seco | F_X5 | Agonist | Yes |
| 30 | Erucastrum gallicum | F_X3 | Agonist | Yes |
| 31 | Erucastrum gallicum | F_X4 | Agonist | Yes |
| 32 | Erucastrum gallicum | F_X5 | Agonist | Yes |
| 33 | Linum usitatissimum | F_X5 | Agonist | Yes |
| 34 | Asclepias syriaca | F_X5 | Agonist | Yes |
| 35 | Hypericum perforatum | F_AX | Agonist | Yes |
| 36 | Hypericum perforatum | F_X1 | Agonist | Yes |
| 37 | Hypericum perforatum | F_X2 | Agonist | Yes |
| 38 | Hypericum perforatum | F_X3 | Agonist | Yes |
| 39 | Hypericum perforatum | F_X4 | Agonist | Yes |
| 40 | Hypericum perforatum | F_X5 | Agonist | Yes |
| 41 | Hypericum perforatum | F_X6 | Agonist | Yes |
| 42 | Larrea tridentata | F_AP | Agonist | Yes |
| 43 | Larrea tridentata | F_AX | Agonist | Yes |
| 44 | Larrea tridentata | F_X0 | Agonist | Yes |
| 45 | Larrea tridentata | F_X2 | Agonist | Yes |
| 46 | Larrea tridentata | F_X3 | Agonist | Yes |
| 47 | Hypericum perforatum | F_X0 | Agonist | Yes |
| 48 | Eucalyptus globulus | F_X0 | Agonist | Yes |
| | subsp. bicostata | | | |
| 49 | Phyllanthus emblica | F_X0 | Agonist | Yes |
| 50 | Phyllanthus emblica | F_X3 | Agonist | Yes |
| 51 | Phyllanthus emblica | F_X4 | Agonist | Yes |
| 52 | Phyllanthus emblica | F_X5 | Agonist | Yes |
| 53 | Eucalyptus globulus | F_AX | Agonist | Yes |
| | subsp. bicostata | | | |
| 54 | Eucalyptus globulus | F_X3 | Agonist | Yes |
| | subsp. bicostata | | | |
| 55 | Eucalyptus globulus | F_X4 | Agonist | Yes |
| | subsp. bicostata | | | |
| 56 | Eucalyptus globulus | F_X5 | Agonist | Yes |
| | subsp. bicostata | | | |
| 57 | Eucalyptus globulus | F_X6 | Agonist | Yes |
| | subsp. bicostata | | | |
| 58 | Veronica polita | F_X3 | Agonist | Yes |
| 59 | Veronica polita | F_X4 | Agonist | Yes |
| 60 | Veronica polita | F_X5 | Agonist | Yes |
| 61 | Alcea rosea | F_X5 | Agonist | Yes |
| 62 | Neolamarckia cadamba | F_X0 | Agonist | Yes |
| 63 | Neolamarckia cadamba | F_X2 | Agonist | Yes |
| 64 | Neolamarckia cadamba | F_X3 | Agonist | Yes |
| 65 | Neolamarckia cadamba | F_X4 | Agonist | Yes |
| 66 | Catharanthus roseus | F_X3 | Agonist | Yes |
| 67 | Catharanthus roseus | F_X4 | Agonist | Yes |
| 68 | Catharanthus roseus | F_X5 | Agonist | Yes |
| 69 | Catharanthus roseus | F_X6 | Agonist | Yes |
| 70 | Podocarpus spicatus | F_AP | Agonist | Yes |
| 71 | Podocarpus spicatus | F_AX | Agonist | Yes |
| 72 | Podocarpus spicatus | F_X2 | Agonist | Yes |
| 73 | Podocarpus spicatus | F_X4 | Agonist | Yes |
| 74 | Podocarpus spicatus | F_X5 | Agonist | Yes |
| 75 | Tripterygium wilfordii | F_AX | Agonist | Yes |
| 76 | Tripterygium wilfordii | F_X0 | Agonist | Yes |
| 77 | Tripterygium wilfordii | F_X2 | Agonist | Yes |
| 78 | Tripterygium wilfordii | F_X3 | Agonist | Yes |
| 79 | Aloe africana | F_X3 | Agonist | Yes |
| 80 | Juniperus monosperma | F_AP | Agonist | Yes |
| 81 | Juniperus monosperma | F_AX | Agonist | Yes |
| 82 | Juniperus monosperma | F_X0 | Agonist | Yes |
| 83 | Juniperus monosperma | F_X2 | Agonist | Yes |
| 84 | Juniperus monosperma | F_X3 | Agonist | Yes |
| 85 | Juniperus monosperma | F_X4 | Agonist | Yes |
| 86 | Juniperus monosperma | F_X5 | Agonist | Yes |
| 87 | Juniperus monosperma | F_X6 | Agonist | Yes |
| 88 | Pleurotus ostreatus | F_X0 | Agonist | Yes |
| 89 | Pleurotus ostreatus | F_X6 | Agonist | Yes |
| 90 | Gypsophila perfoliata | F_X2 | Agonist | Yes |
| 91 | Gypsophila perfoliata | F_X3 | Agonist | Yes |
| 92 | Gypsophila perfoliata | F_X4 | Agonist | Yes |
| 93 | Clausena excavata | F_AP | Agonist | Yes |
| 94 | Clausena excavata | F_AX | Agonist | Yes |
| 95 | Clausena excavata | F_X3 | Agonist | Yes |
| 96 | Clausena excavata | F_X4 | Agonist | Yes |
| 97 | Clausena excavata | F_X5 | Agonist | Yes |
| 98 | Gynura procumbens | F_X2 | Agonist | Yes |
| 99 | Trifolium pratense | F_X3 | Agonist | Yes |
| | cv. Redman | | | |
| 100 | Trifolium pratense | F_X4 | Agonist | Yes |
| | cv. Redman | | | |
| 101 | Trifolium pratense | F_X5 | Agonist | Yes |
| | cv. Redman | | | |
| 102 | Rosmarinus officinalis | F_AP | Agonist | Yes |
| 103 | Rosmarinus officinalis | F_AX | Agonist | Yes |
| 104 | Rosmarinus officinalis | F_X3 | Agonist | Yes |
| 105 | Rosmarinus officinalis | F_X4 | Agonist | Yes |
| 106 | Rosmarinus officinalis | F_X5 | Agonist | Yes |
| 107 | Nelumbo nucifera | F_X3 | Agonist | Yes |
| 108 | Nelumbo nucifera | F_X4 | Agonist | Yes |
| 109 | Citrus unshiu | F_X0 | Agonist | Yes |
| 110 | Citrus unshiu | F_X1 | Agonist | Yes |
| 111 | Citrus unshiu | F_X2 | Agonist | Yes |
| 112 | Citrus unshiu | F_X3 | Agonist | Yes |
| 113 | Hylocomium splendens | F_X4 | Agonist | Yes |
| 114 | Citrus sinensis | F_X3 | Agonist | Yes |
| 115 | Citrus sinensis | F_X4 | Agonist | Yes |
| 116 | Citrus sinensis | F_X5 | Agonist | Yes |
| 117 | Juniperus drupacea | F_AX | Agonist | Yes |
| 118 | Juniperus drupacea | F_X3 | Agonist | Yes |
| 119 | Juniperus drupacea | F_X4 | Agonist | Yes |
| 120 | Juniperus drupacea | F_X5 | Agonist | Yes |
| 121 | Juniperus drupacea | F_X6 | Agonist | Yes |
| 122 | Forsythia suspensa | F_AP | Agonist | Yes |
| 123 | Forsythia suspensa | F_AX | Agonist | Yes |
| 124 | Forsythia suspensa | F_X3 | Agonist | Yes |
| 125 | Forsythia suspensa | F_X4 | Agonist | Yes |
| 126 | Forsythia suspensa | F_X5 | Agonist | Yes |

TABLE 6

| | | Fraction | Modulation | Bioactivity |
|---|---|---|---|---|
| | Plant | ID | Type | (in vitro) |
| 1 | Medicago sativa | F_X6 | PAM | Yes |
| | subsp. sativa | | | |
| 2 | Solanum americanum | F_X4 | PAM | Yes |
| 3 | Coffea arabica | F_AP | PAM | Yes |
| 4 | Coffea arabica | F_X3 | PAM | Yes |
| 5 | Coffea arabica | F_X4 | PAM | Yes |
| 6 | Coffea arabica | F_X5 | PAM | Yes |
| 7 | Coffea arabica | F_X6 | PAM | Yes |
| 8 | Momordica charantia | F_X3 | PAM | Yes |
| 9 | Momordica charantia | F_X5 | PAM | Yes |
| 10 | Aloe vera | F_AX | PAM | Yes |
| 11 | Aloe vera | F_X0 | PAM | Yes |
| 12 | Aloe vera | F_X1 | PAM | Yes |
| 13 | Aloe vera | F_X2 | PAM | Yes |
| 14 | Aloe vera | F_X3 | PAM | Yes |
| 15 | Aloe vera | F_X4 | PAM | Yes |
| 16 | Aloe vera | F_X5 | PAM | Yes |
| 17 | Aloe vera | F_X6 | PAM | Yes |
| 18 | Silybum marianum | F_X3 | PAM | Yes |
| 19 | Theobroma cacao | F_X6 | PAM | Yes |
| 20 | Aloe ferox | F_AP | PAM | Yes |
| 21 | Aloe ferox | F_AX | PAM | Yes |
| 22 | Aloe ferox | F_BX | PAM | Yes |
| 23 | Aloe ferox | F_X1 | PAM | Yes |
| 24 | Aloe ferox | F_X2 | PAM | Yes |
| 25 | Aloe ferox | F_X3 | PAM | Yes |
| 26 | Aloe ferox | F_X4 | PAM | Yes |
| 27 | Aloe ferox | F_X5 | PAM | Yes |
| 28 | Aloe ferox | F_X6 | PAM | Yes |
| 29 | Gardenia jasminoides | F_X2 | PAM | Yes |
| 30 | Gardenia jasminoides | F_X3 | PAM | Yes |
| 31 | Gardenia jasminoides | F_X4 | PAM | Yes |
| 32 | Gardenia jasminoides | F_X5 | PAM | Yes |
| 33 | Gardenia jasminoides | F_X6 | PAM | Yes |
| 34 | Anacardium occidentale | F_X1 | PAM | Yes |
| 35 | Anacardium occidentale | F_X2 | PAM | Yes |
| 36 | Anacardium occidentale | F_X3 | PAM | Yes |
| 37 | Anacardium occidentale | F_X6 | PAM | Yes |
| 38 | Capsicum annuum | F_X3 | PAM | Yes |
| 39 | Capsicum annuum | F_X4 | PAM | Yes |
| 40 | Capsicum annuum | F_X5 | PAM | Yes |
| 41 | Capsicum annuum | F_X6 | PAM | Yes |
| 42 | Paeonia lactiflora | F_X2 | PAM | Yes |
| 43 | Paeonia lactiflora | F_X6 | PAM | Yes |
| 44 | Rheum officinale | F_AX | PAM | Yes |
| 45 | Rheum officinale | F_X3 | PAM | Yes |
| 46 | Rheum officinale | F_X6 | PAM | Yes |
| 47 | Chaenomeles sinensis | F_X2 | PAM | Yes |
| 48 | Chaenomeles sinensis | F_X3 | PAM | Yes |
| 49 | Chaenomeles sinensis | F_X5 | PAM | Yes |
| 50 | Chaenomeles sinensis | F_X6 | PAM | Yes |
| 51 | Citrus clementina | F_AP | PAM | Yes |
| 52 | Citrus clementina | F_AX | PAM | Yes |
| 53 | Citrus clementina | F_X3 | PAM | Yes |
| 54 | Citrus clementina | F_X4 | PAM | Yes |
| 55 | Citrus clementina | F_X5 | PAM | Yes |
| 56 | Citrus clementina | F_X6 | PAM | Yes |
| 57 | Hordeum vulgare | F_X6 | PAM | Yes |
| | cv. Seco | | | |
| 58 | Erucastrum gallicum | F_AX | PAM | Yes |
| 59 | Erucastrum gallicum | F_X5 | PAM | Yes |
| 60 | Erucastrum gallicum | F_X6 | PAM | Yes |
| 61 | Linum usitatissimum | F_X4 | PAM | Yes |
| 62 | Linum usitatissimum | F_X5 | PAM | Yes |
| 63 | Linum usitatissimum | F_X6 | PAM | Yes |
| 64 | Asclepias syriaca | F_AP | PAM | Yes |
| 65 | Asclepias syriaca | F_AX | PAM | Yes |
| 66 | Asclepias syriaca | F_X5 | PAM | Yes |
| 67 | Avena sativa | F_AP | PAM | Yes |
| 68 | Avena sativa | F_AX | PAM | Yes |
| 69 | Avena sativa | F_X0 | PAM | Yes |
| 70 | Avena sativa | F_X2 | PAM | Yes |
| 71 | Avena sativa | F_X3 | PAM | Yes |
| 72 | Avena sativa | F_X4 | PAM | Yes |
| 73 | Avena sativa | F_X5 | PAM | Yes |
| 74 | Avena sativa | F_X6 | PAM | Yes |
| 75 | Hypericum perforatum | F_AX | PAM | Yes |
| 76 | Hypericum perforatum | F_X1 | PAM | Yes |
| 77 | Hypericum perforatum | F_X2 | PAM | Yes |
| 78 | Hypericum perforatum | F_X3 | PAM | Yes |
| 79 | Hypericum perforatum | F_X5 | PAM | Yes |
| 80 | Hypericum perforatum | F_X6 | PAM | Yes |
| 81 | Curcuma longa | F_X0 | PAM | Yes |
| 82 | Curcuma longa | F_X1 | PAM | Yes |
| 83 | Curcuma longa | F_X2 | PAM | Yes |
| 84 | Curcuma longa | F_X6 | PAM | Yes |
| 85 | Zea mays | F_X3 | PAM | Yes |
| 86 | Zea mays | F_X4 | PAM | Yes |
| 87 | Zea mays | F_X5 | PAM | Yes |
| 88 | Larrea tridentata | F_AP | PAM | Yes |
| 89 | Larrea tridentata | F_AX | PAM | Yes |
| 90 | Larrea tridentata | F_X0 | PAM | Yes |
| 91 | Larrea tridentata | F_X1 | PAM | Yes |
| 92 | Larrea tridentata | F_X2 | PAM | Yes |
| 93 | Larrea tridentata | F_X3 | PAM | Yes |
| 94 | Larrea tridentata | F_X4 | PAM | Yes |
| 95 | Larrea tridentata | F_X5 | PAM | Yes |
| 96 | Larrea tridentata | F_X6 | PAM | Yes |
| 97 | Nicotiana tabacum | F_AX | PAM | Yes |
| 98 | Nicotiana tabacum | F_X3 | PAM | Yes |
| 99 | Nicotiana tabacum | F_X4 | PAM | Yes |
| 100 | Nicotiana tabacum | F_X6 | PAM | Yes |
| 101 | Hypericum perforatum | F_X0 | PAM | Yes |
| 102 | Rheum officinale | F_X0 | PAM | Yes |
| 103 | Eucalyptus globulus | F_X0 | PAM | Yes |
| | subsp. bicostata | | | |
| 104 | Musa acuminata | F_AX | PAM | Yes |
| 105 | Musa acuminata | F_X1 | PAM | Yes |
| 106 | Musa acuminata | F_X3 | PAM | Yes |
| 107 | Musa acuminata | F_X5 | PAM | Yes |
| 108 | Solanum melongena | F_X0 | PAM | Yes |
| 109 | Solanum melongena | F_X1 | PAM | Yes |
| 110 | Solanum melongena | F_X2 | PAM | Yes |
| 111 | Solanum melongena | F_X3 | PAM | Yes |
| 112 | Solanum melongena | F_X6 | PAM | Yes |
| 113 | Phyllanthus emblica | F_X0 | PAM | Yes |
| 114 | Phyllanthus emblica | F_X1 | PAM | Yes |
| 115 | Phyllanthus emblica | F_X2 | PAM | Yes |
| 116 | Phyllanthus emblica | F_X3 | PAM | Yes |
| 117 | Phyllanthus emblica | F_X4 | PAM | Yes |
| 118 | Phyllanthus emblica | F_X5 | PAM | Yes |
| 119 | Phyllanthus emblica | F_X6 | PAM | Yes |
| 120 | Eucalyptus globulus | F_X4 | PAM | Yes |
| | subsp. bicostata | | | |
| 121 | Eucalyptus globulus | F_X5 | PAM | Yes |
| | subsp. bicostata | | | |
| 122 | Eucalyptus globulus | F_X6 | PAM | Yes |
| | subsp. bicostata | | | |
| 123 | Solanum lycopersicum | F_X3 | PAM | Yes |
| 124 | Solanum lycopersicum | F_X4 | PAM | Yes |
| 125 | Veronica polita | F_X0 | PAM | Yes |
| 126 | Veronica polita | F_X3 | PAM | Yes |
| 127 | Veronica polita | F_X5 | PAM | Yes |
| 128 | Alcea rosea | F_AX | PAM | Yes |
| 129 | Alcea rosea | F_X0 | PAM | Yes |
| 130 | Alcea rosea | F_X5 | PAM | Yes |
| 131 | Plukenetia volubilis | F_X5 | PAM | Yes |
| 132 | Euphorbia tirucalli | F_X6 | PAM | Yes |
| 133 | Neolamarckia cadamba | F_X0 | PAM | Yes |
| 134 | Neolamarckia cadamba | F_X2 | PAM | Yes |
| 135 | Neolamarckia cadamba | F_X3 | PAM | Yes |
| 136 | Citrus limon | F_AP | PAM | Yes |
| 137 | Citrus limon | F_CX | PAM | Yes |
| 138 | Citrus limon | F_X2 | PAM | Yes |
| 139 | Citrus limon | F_X3 | PAM | Yes |
| 140 | Citrus limon | F_X4 | PAM | Yes |
| 141 | Citrus limon | F_X5 | PAM | Yes |
| 142 | Citrus limon | F_X6 | PAM | Yes |
| 143 | Catharanthus roseus | F_X2 | PAM | Yes |
| 144 | Catharanthus roseus | F_X3 | PAM | Yes |
| 145 | Catharanthus roseus | F_X6 | PAM | Yes |
| 146 | Mirabilis jalapa | F_X2 | PAM | Yes |
| 147 | Mirabilis jalapa | F_X3 | PAM | Yes |
| 148 | Mirabilis jalapa | F_X4 | PAM | Yes |
| 149 | Mirabilis jalapa | F_X5 | PAM | Yes |
| 150 | Mirabilis jalapa | F_X6 | PAM | Yes |
| 151 | Podocarpus spicatus | F_AP | PAM | Yes |
| 152 | Podocarpus spicatus | F_AX | PAM | Yes |
| 153 | Podocarpus spicatus | F_BX | PAM | Yes |
| 154 | Podocarpus spicatus | F_X2 | PAM | Yes |
| 155 | Podocarpus spicatus | F_X3 | PAM | Yes |
| 156 | Podocarpus spicatus | F_X4 | PAM | Yes |
| 157 | Tripterygium wilfordii | F_AX | PAM | Yes |
| 158 | Tripterygium wilfordii | F_X0 | PAM | Yes |
| 159 | Spatholobus suberectus | F_X1 | PAM | Yes |
| 160 | Tripterygium wilfordii | F_X1 | PAM | Yes |
| 161 | Spatholobus suberectus | F_X2 | PAM | Yes |
| 162 | Tripterygium wilfordii | F_X2 | PAM | Yes |
| 163 | Aloe africana | F_X2 | PAM | Yes |
| 164 | Spatholobus suberectus | F_X3 | PAM | Yes |
| 165 | Tripterygium wilfordii | F_X3 | PAM | Yes |
| 166 | Aloe africana | F_X3 | PAM | Yes |
| 167 | Aloe africana | F_X5 | PAM | Yes |
| 168 | Spatholobus suberectus | F_X6 | PAM | Yes |
| 169 | Aloe africana | F_X6 | PAM | Yes |
| 170 | Crinum moorei | F_X6 | PAM | Yes |
| 171 | Juniperus monosperma | F_AP | PAM | Yes |
| 172 | Juniperus monosperma | F_AX | PAM | Yes |
| 173 | Juniperus monosperma | F_X0 | PAM | Yes |
| 174 | Juniperus monosperma | F_X2 | PAM | Yes |
| 175 | Juniperus monosperma | F_X3 | PAM | Yes |
| 176 | Juniperus monosperma | F_X4 | PAM | Yes |
| 177 | Juniperus monosperma | F_X5 | PAM | Yes |
| 178 | Juniperus monosperma | F_X6 | PAM | Yes |
| 179 | Pleurotus ostreatus | F_X0 | PAM | Yes |
| 180 | Pleurotus ostreatus | F_X6 | PAM | Yes |
| 181 | Gypsophila perfoliata | F_X0 | PAM | Yes |
| 182 | Gypsophila perfoliata | F_X2 | PAM | Yes |
| 183 | Gypsophila perfoliata | F_X3 | PAM | Yes |
| 184 | Gypsophila perfoliata | F_X4 | PAM | Yes |
| 185 | Clausena excavata | F_AX | PAM | Yes |
| 186 | Clausena excavata | F_X0 | PAM | Yes |
| 187 | Clausena excavata | F_X4 | PAM | Yes |
| 188 | Clausena excavata | F_X5 | PAM | Yes |
| 189 | Clausena excavata | F_X6 | PAM | Yes |
| 190 | Gynura procumbens | F_AX | PAM | Yes |
| 191 | Gynura procumbens | F_X2 | PAM | Yes |
| 192 | Gynura procumbens | F_X3 | PAM | Yes |
| 193 | Gynura procumbens | F_X6 | PAM | Yes |
| 194 | Trifolium pratense | F_X3 | PAM | Yes |
| | cv. Redman | | | |
| 195 | Trifolium pratense | F_X4 | PAM | Yes |
| | cv. Redman | | | |
| 196 | Trifolium pratense | F_X5 | PAM | Yes |
| | cv. Redman | | | |
| 197 | Oryza sativa | F_X5 | PAM | Yes |
| 198 | Rosmarinus officinalis | F_AP | PAM | Yes |
| 199 | Rosmarinus officinalis | F_AX | PAM | Yes |
| 200 | Rosmarinus officinalis | F_X3 | PAM | Yes |
| 201 | Rosmarinus officinalis | F_X4 | PAM | Yes |
| 202 | Rosmarinus officinalis | F_X5 | PAM | Yes |
| 203 | Rosmarinus officinalis | F_X6 | PAM | Yes |
| 204 | Nelumbo nucifera | F_X0 | PAM | Yes |
| 205 | Nelumbo nucifera | F_X3 | PAM | Yes |
| 206 | Nelumbo nucifera | F_X4 | PAM | Yes |
| 207 | Carthamus tinctorius | F_X1 | PAM | Yes |
| 208 | Carthamus tinctorius | F_X6 | PAM | Yes |
| 209 | Citrus unshiu | F_AX | PAM | Yes |
| 210 | Citrus unshiu | F_X0 | PAM | Yes |
| 211 | Citrus unshiu | F_X1 | PAM | Yes |
| 212 | Citrus unshiu | F_X2 | PAM | Yes |
| 213 | Citrus unshiu | F_X3 | PAM | Yes |
| 214 | Hylocomium splendens | F_X4 | PAM | Yes |
| 215 | Hylocomium splendens | F_X5 | PAM | Yes |
| 216 | Citrus sinensis | F_X3 | PAM | Yes |
| 217 | Citrus sinensis | F_X4 | PAM | Yes |
| 218 | Juniperus drupacea | F_AP | PAM | Yes |
| 219 | Juniperus drupacea | F_AX | PAM | Yes |
| 220 | Juniperus drupacea | F_X3 | PAM | Yes |
| 221 | Juniperus drupacea | F_X4 | PAM | Yes |
| 222 | Juniperus drupacea | F_X5 | PAM | Yes |
| 223 | Juniperus drupacea | F_X6 | PAM | Yes |
| 224 | Forsythia suspensa | F_AP | PAM | Yes |
| 225 | Forsythia suspensa | F_AX | PAM | Yes |
| 226 | Forsythia suspensa | F_X0 | PAM | Yes |
| 227 | Forsythia suspensa | F_X2 | PAM | Yes |
| 228 | Forsythia suspensa | F_X3 | PAM | Yes |
| 229 | Forsythia suspensa | F_X4 | PAM | Yes |
| 230 | Forsythia suspensa | F_X5 | PAM | Yes |

### Example 5

This example describes in vitro data of compounds of the disclosure in a standardized bioassay protocol.

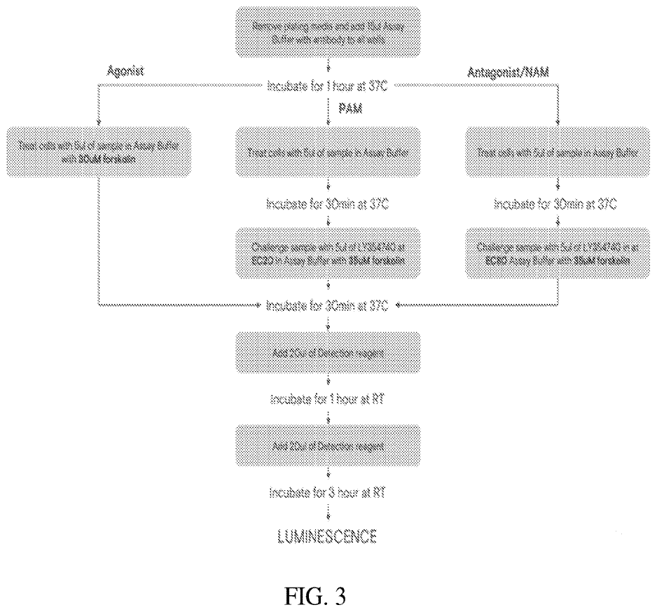

A standardized bioassay protocol was followed as illustrated in FIG. 3. In the standardized bioassay protocol, an assay buffer of HBSS with 1% HEPES was used. Cells were seeded overnight at 10,000 cells/well in 20 μl plating reagent. Controls for the experiment were an agonist of L-glutamic acid (AC156212500), agonist of LY354740 (fisher part #: 32-465-0), antagonist of LY341495 (fisher part #: 12-095-0), PAM of BINA (fisher part #: 40-485-0), and NAM of (decoglurant (fisher part #: 50-203-0742).

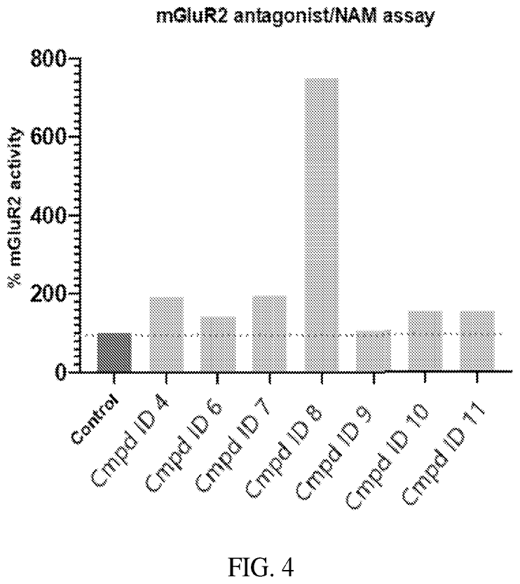

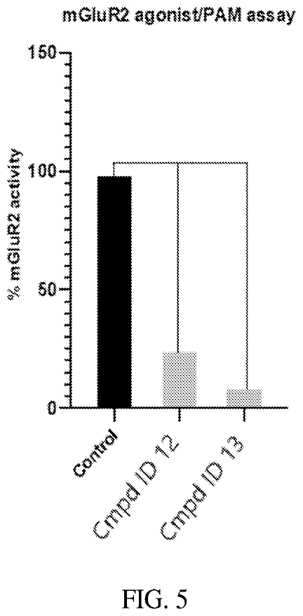

The results are illustrated in Table 7 and FIGS. 4 and 5. FIG. 4 illustrates mGlu2 activity greater than control (dotted red line at 100%) is indicative of an mGlu2 negative allosteric modulator or antagonist. The assay was performed at 20 μM. FIG. 5 illustrates mGlu2 activity less than the control (at 100%) is indicative of a mGlu2 positive allosteric modulator or agonist. The assay was performed at 100 μM.

TABLE 7

| Compound ID | Bioactivity Class | Activity Level |
|---|---|---|
| 4 | Antagonist/NAM | 20 μM/191.200 |
| 6 | Antagonist/NAM | 20 μM/142.558 |
| 7 | Antagonist/NAM | 20 μM/195.543 |
| 8 | Antagonist/NAM | 20 μM/749.225 |
| 9 | Antagonist/NAM | 20 μM/107.501 |
| 10 | Antagonist/NAM | 20 μM/152.609 |
| 11 | Antagonist/NAM | 20 μM/153.003 |
| 12 | Agonist/PAM | 100 μM/25.688 |
| 13 | Agonist/PAM | 100 μM/9.870 |

### Example 6

This example describes a research protocol for testing the tolerability and efficacy of novel plant-derived supplements on disturbed sleep and anxiety.

### Background and Objectives

Emotional and physical stressors disturb sleep, physiological rhythms, and increase the risk of developing mood disorders. Considering recent evidence that repeated or chronic use of sedative-hypnotic prescription or over-the-counter soporific drugs negatively impact brain health, there is a need to discover novel natural compounds that are safe and effective in reducing emotional stress and protecting sleep and diurnal rhythms. The primary objective of this example will be to test the hypothesis that oral ingestion of nutraceutical supplements of an embodiment of the disclosure reduces the negative effects of emotional stress on sleep, physiological rhythms, and mood.

### Primary Objective-Experiment 1: Maximum Tolerable Dose (MTD) Study

The tolerability of two potential compounds of the disclosure will be tested. Compounds will be administered using an oral dosing technique. Rats will be administered compounds once per day for 4 days. Doses will be based on pharmacology profile. Additional dependent measures are informed by published MTD guidelines. Body weight and food intake will be measured daily. Rats will be sacrificed at the end of the study and tissues will be dissected, inspected, weighed and frozen for future potential toxicology-related analyses. Continuous infrared video home cage surveillance will capture changes in behavior, including sickness behavior. The results of this work will help to identify MTD for each compound to be tested in the efficacy “Stress and biotelemetry” study described below.

### Experimental Design

2×2 (male, female)×4 to 7 doses+vehicle: 16 to 28 groups, estimated number of rats ranges from 160 to 280, depending on MTD range. Shortly after the start of the inactive/sleep cycle (0900), rats will receive daily oral administration of vehicle (vehicle) or one of the compounds of the disclosure for 5 days. Due the labor intensity of the project, multiple balanced cohorts will be tested with staggered start dates. Study Duration: 18 days (14 days vivarium habituation, 4 days oral administration of vehicle and compound×4 cohorts ~20 weeks.

### Experiment 2: Efficacy Study: Stress and Biotelemetry

Using the rat CCF model of chronic emotional stress described below, daily oral ingestion of the compounds of the disclosure will be tested daily and the negative effects of emotional stress on sleep, physiological rhythms, and anxiety will be measured. The Maximal Tolerable Dose (MTD) of the compounds of the disclosure will be tested and based on the MTD study described above. Chronic Conditioned Fear (CCF) is a validated model of sustained emotional stress previously reported to produces a sequala of adaptation failures beginning with disturbed sleep, flattened physiological rhythms, and anxiety-like behavior. Dependent measures will be collected using in vivo biotelemetry and infrared Synology home cage surveillance. Rats will be randomly assigned to be exposed to the chronic conditioned fear protocol (CCF) or remain in the home cages and serve as non-stressed controls.

All rats will be surgically implanted with HD-SO2 transmitters for biotelemetry which allows measurement of Locomotor Activity (LA), Heart Rate (HR), Core Body Temperature (CBT) and electromyography of dorsal neck muscle (EMG), and electroencephalogram (EEG). For this project, rats will be instrumented with telemetry to measure LA, CBT, EMG, and EEG. After surgical recovery, continuous biotelemetry measures will be collected prior to CCF (24 h per day for 3 days, home cage recording), during CCF (20-min per session, 2 CF sessions per day for 21 days, conditioned fear chamber), and after CCF (24 h per day for 7 days, home cage recording). For this project, both male and female rats will be tested because there are reported sex differences in conditioned fear.

### Secondary Objectives

These studies will also address the potential impacts of oral ingestion of compounds of the disclosure on behavior and physiology in the absence of stress. The Maximum Tolerable Dose Study will include home cage surveillance to determine the impact that compounds on home cage behavior. And because the Stress and Biotelemetry Study includes non-stressed controls, the study will also discover any impacts of repeated oral ingestion of compounds on sleep, in vivo physiology, and behavior.

### Experimental Approach Foundation and Feasibility

Conditioned Fear (CF): The CF is produced when rats are conditioned to fear a specific environment by placing them in a fear conditioning chamber and administering 3, 2-sec, 1.5 mA footshocks. A single pairing of footshock with the chamber will condition the rats to fear the chamber without additional footshock. This is called “conditioned fear” (CF). Rodents display fear by freezing, which is defined as the absence of movement except that required for respiration. CF also produces classic features of the in vivo stress response, including elevated heart rate, blood pressure, and core body temperature.

The Chronic Conditioned Fear (CCF): The CCF model is designed to explore the mental and physical impacts of sustained emotional stress. For the CCF model, rats are exposed to the conditioned chamber for 20 min, twice per day, at random times during their sleep cycle. Fear behavior will extinguish after several exposures to the chamber in the absence of footshock. If the time spent freezing during the 20 min test falls below 50%, a single “reminder” footschook (2-sec, 1.5 mA) will restore the fear behavior and stress responses. Typically, only 1 or 2 “reminder” footshocks are required to sustain fear behavior and stress responses for 14 days (2× per day). The Fleshner stress physiology lab is expert in this procedure and have previously reported that conditioned fear triggers features of the in vivo stress response, including elevations in HR, MAP, CBT and chronic conditioned fear produces sleep disturbance, diurnal rhythm flattening, and anxiety-like behavior. In humans, repeated psychological stressors like fear also increase HR, CBT, and anxiety, adding to the translational strength of this model.

Home cage behavioral pattern indicative of anxiety: Stressor exposure changes in patterns of home cage behavior suggestive of increased anxiety and depression. Infrared cameras will capture 24 h video recordings of home cage behavior. Behavior will be analyzed before, during, and after stressor exposure. Changes in anxiety and depression will be captured by analyzing home cage behavior using Behavioral Observation Research Interactive Software (BORIS) analyzes of home cage behavior rather than open field, forced swim-test, or radial-arm maze tasks because evidence suggests that these tests by themselves disturb sleep, flatten diurnal rhythms, and are influenced by the presence of the observer.

### Methods

Biotelemetry (DSI™)—In vivo Stress Responses, Diurnal Rhythms, and Sleep EEG: Sleep (rapid eye movement, REM and non-rapid eye movement, NREM) will be measured using EMG and EEG data and analyzed using Neuroscore™ software. Diurnal rhythm flattening will be captured using LA, EEG, and CBT data and scored using the ClockLab program version 6.1.02 (Actimetrics Software™, Wilmette, IL, USA).

Infrared Synology Home cage Surveillance: Due to video equipment limitations and time constraints, rats in the Tolerance Study but not the Stress and Biotelemetry study will be video recorded in their home cages using the system described. This non-invasive procedure does not disrupt the animals. Axis Model #M3104 infrared cameras are placed in proximity to the rats' home cages and allows side-to-side video 24 h recordings of home cage behavior. These cameras provide excellent visual resolution of the animals during the light and dark cycles, introduce no light, and do not disturb sleep or diurnal rhythmicity of sleep and physiological rhythms. Synology Surveillance Station will be used to acquire our video recordings. Behavioral Observation Research Interactive Software (BORIS) will be used to score home cage video recordings (Friard and Gamba, 2016). This software allows one to simultaneously score multiple behaviors. Snapshot Analyses: Home cage behavior is scored 1 h prior to and 1 h after lights off and presented as % of control (total time of a particular activity control animals (Vehicle, HCC/total time of a particular activity in rats exposed to emotional stress). This window of time captures activity that is motivated by the light/dark change. It is well-established that in the hour after lights off rats' display a high-level of spontaneous activity; and feeding and drinking occurs nearly exclusively in the dark (active) cycle. The following is a detailed description of behaviors to be scored (duration, frequency) per the Stanford Ethogram definitions: Inactive Behavior: “The absence of movement” and includes period when animals are “still and alert” and “still and not alert suggestive of sleep state.” Overall Activity: “Any behavior that involves the presence of movement.” The total duration (in addition to frequency) will be scored. Active Behaviors will also be broken down into two subcategories “Maintenance Behavior” or “General Activity.” Maintenance Behavior: Behaviors that typically function to maintain the physiological stasis, comfort, and appearance of the animal and includes drinking, feeding, grooming (self-grooming), and nesting behavior. General Activity: Other behaviors that involve the presence of movement but do not fall into the “Maintenance Behavior” category and include exploratory behavior, affiliative interactions (allogrooming, wrestling/playing), agonistic interactions, and miscellaneous behaviors.

Expected Outcomes: Based on previous results, rats exposed to CCF will have disturbed sleep/wake behavior, sleep architecture, and diurnal LA, CBT, and EEG rhythms. The following are strengths of this experimental approach: emotional stressor, conditioned fear (CF), rigorous and reproducible outcomes of the chronic conditioned fear (CCF) model, automated measures of behavior, physiology, and sleep, real-time, continuous, hands-off measurements of fear behavior and in vivo stress physiology, and ethological approach to determine emotional/behavioral impacts of stressor exposure (24 h video recording of home cage behavior and BORIS analyses).

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of

A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A use of one or more compounds of Formula (I): 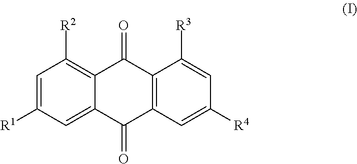
or a salt thereof in the manufacture of a composition for modulating a metabotropic glutamate receptor;
wherein R¹, R², R³, and R⁴ are each independently selected from hydrogen, deuterium, hydroxyl, halogen, keto, optionally substituted ester, optionally substituted aryl, optionally substituted alkoxy, optionally substituted C₁-C₆-alkyl, optionally substituted —O—C₁-C₆-alkyl, or optionally substituted C₃-C₆ cycloalkyl.

2. (canceled)

3. The use of claim 1, wherein R¹ of the compound is hydrogen or hydroxyl.

4. The use of claim 1, wherein R² of the compound is hydroxyl or keto.

5. The use of claim 1, wherein R³ of the compound is hydroxyl or keto.

6. The use of claim 1, wherein R⁴ of the compound is hydrogen, hydroxyl, keto, optionally substituted O—C₁-C₆-alkyl, or optionally substituted C₁-C₆-alkyl.

7. A use of one or more compounds selected from: 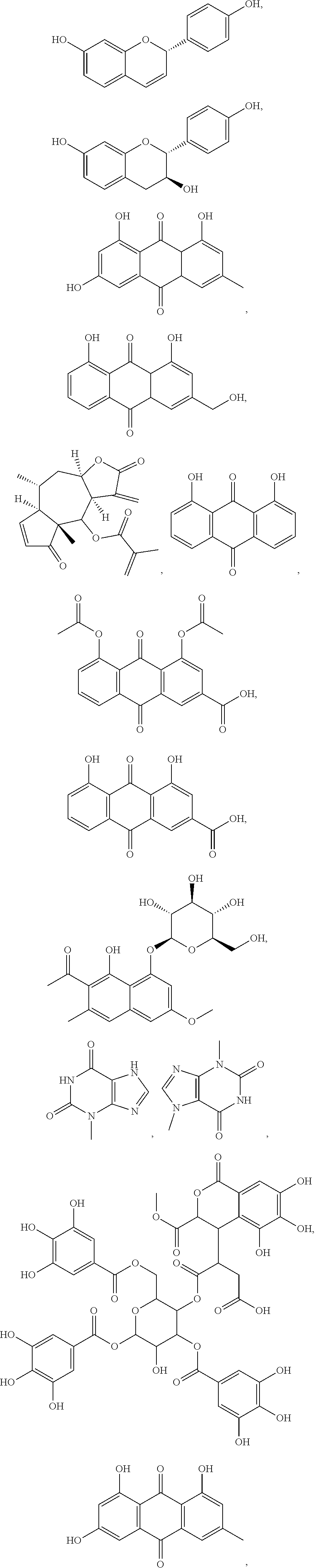
or a combination thereof in the manufacture of a composition for modulating a metabotropic glutamate receptor.

8. The use of claim 7, wherein the metabotropic glutamate receptor is a Group II receptor.

9. The use of claim 7, wherein the one or more compounds is from a plant extract.

10. The use of claim 9, wherein the plant extract is selected from the group consisting of Rheum officinale, Phyllanthus emblica, Malus domestica, Tripterygium wilfordii, Aloe barbadensis, Theobroma cacao, Chaenomeles sinensis, Hypericum perforatum, Citrus unshiu, Nelumbo nucifera, Acacia luederitzii, Cassia abbreviate, Cassia fistula, Colophospermum mopane, Julbernardia globiflora, Habranthus brachyandrus, Monactis macbridei, Arnica acaulis, Arnica montana, Daucus carota, Bauhinia manca, Botrytis cinerea, Brosimum acutifolium, Broussonetia kazinoki, Broussonetia papyrifera, Dalbergia latifolia, Dracaena cambodiana, Dracaena draco, Narcissus pseudonarcissus, Phanera guianensis, Rohdea chinensis, Soymida febrifuga, Taxus brevifolia, Tupistra chinensis, Abri herba, Abrus pulchellus subsp. Cantoniensis, Achyranthes bidentata, Aconitum seravschanicum, Aconitum tasiromontanum, Acori tataninowii rhizome, Acorus gramineus, Acorus tatarinowii, Actinidia chinensis planch, Adansonia grandidieri, Aframomum giganteum, Aizopsis selkiana, Albizia myriophylla, Alisma plantago-aquatica subsp. orientale, Aloe castellorum, Aloe vera, Alpiniae officinarum rhizome, Amaranthus hypochondriacus, Ampelopsis japonica, Amphorogyne spicata, Anaphalis lacteal, Anchusa azurea, Andrographis laxiflora, Andrographis neesiana, Anemarrhena asphodeloides, Aphyllocladus denticulatus, Aralia decaisneana, Artemisia annua, Artemisia parviflora, Artemisia scoparia [Syn. Artemisia capillaris v, Artemisia scoparia, Aspidosperma discolor, Asteris radix et rhizome, Aster sedifolius, Aster tataricus, Atadinus libanoticus, Baccharis potosina, Balanophora laxiflora, Bassia muricata, Benincasa hispida, Beta vulgaris vulgaris, Bistorta elliptica, Bistorta officinalis, Boehmeriae rhizoma et radix, Boehmeria nivea, Boehmeria platanifolia [Syn. Boehmeria tricuspis, Boehmeria platanifolia, Bolbostemae paniculati bulbus, Bolbostemma paniculatum, Brassica rapa, Bridelia stipularis, Canarium bengalense, Carduus tenuiflorus, Cascara sagrada, Cassia alata L., Cassia angolensis, Cassiae semen, Cassia fastuosa, Cassia javanica, Cassia obtusifolia, Cassia occidentalis, Cassia speciosa, Cassia tora L., Cassia tora, Cassia torosa L., Catalpa longissima, Caulerpa peltata, Caulis Polygoni Multiflori, Caulis Sargentodoxae, Caulocystis cephalornithos, Cephaelis ipecacuanha, Cephalotaxus griffithii, Chaiturus marrubiastrum, Chamaecrista mimosoides, Chamaecrista nigricans, Chamaecrista nomame, Chamaecrista pumila, Cheilanthes argentea, Chenopodium quinoa, Cichorium intybus, Cirsium oligophyllum, Clibadium sprucei, Coreopsis tinctoria, Corydalis rhizoma, Cranfillia fluviatilis, Cratoxylum formosum, Cremastra appendiculata, Cynanchum viminale, Dalbergia hupeana, Dalbergia lanceolaria subsp. paniculata, Degenia velebitica, Delphinium nuttallianum, Delphinium virgatum, Dendriopoterium menendezii, Dendrobium thyrsiflorum, Derris canarensis, Derris cuneifolia, Derris oblonga, Dianthi herba, Dianthus caryophyllus, Dictyopteris australis, Dillenia indica, Dioscoreae bulbiferae, Dipsacus azureus, Dodonaea viscosa, Dysoxylum cumingianum, Dysoxylum muelleri, Elymus repens, Enceliopsis argophylla, Endophytic aspergillus, Epimedium koreanum, Epimrdii herba, Eria mollis, Eriocaulon buergerianum, Eriocaulon sieboldianum, Erythrina lysistemon, Erythrophleum ivorense, Euodia bodinieri, Euodia officinalis, Euodia ruticarpa, Fagopyrum esculentum, Fagopyrum tataricum, Fallopia multiflora, Fallopia sachalinensis, Flacourtia jangomas, Frangula alnus, Frangula purshiana, Fridericia triplinervia, Garcinia polyantha, Gleichenia quadripartita, Glycyrrhiza uralensis, Gnaphalii affinis herba, Grangea maderaspatana, Hedera helix, Helichrysum chionosphaerum, Helichrysum plicatum, Heynea trijuga, Hoveniae dulcis semen, Hymenoxys lemmonii, Hypericum prolificum, Hypericum sampsonii, Isatdis radix, Isatis tinctoria, Jacquemontia paniculate, Juniperus formosana, Justicia secunda, Lactuca plumieri, Lactuca sativa, Lanaria lanata, Larix kaempferior, Lavandula vera, Leptocereus quadricostatus, Lespedeza tomentosa, Ligularia pleurocaulis, Lilii bulbus, Lilium brownii f. e. brown var. colchesteri wils, Lilium brownii var. viridulum [Syn. Lilium brown, Lilium brownii var. viridulum, Lobelia berlandieri, Loranthus pentandrus, Lupinus corymbosus, Lycii cortex, Maclura tinctoria, Magnolia figo, Magnolia ovata, Mandragora autumnalis, Melicope triphylla, Mentha arvensis, Mentha canadensis, Menthae herba, Metrodorea nigra, Miliusa velutina, Mitracarpus scaber, Muehlenbeckia tamnifolia, Muntingia calabura, Myrsine africana, Nekemias grossedentata, Neobalanocarpus heimii, Nepeta granatensis, Nepeta parnassica, Nuxia floribunda, Onobrychis arenaria, Oryctes monoceros, Osmanthus armatus, Passiflora sexflora, Persicaria decipiens, Persicaria hydropiper, Persicaria maculosa, Persicaria perfoliata, Persicaria salicifolia, Petunia integrifolia subsp. inflata, Phaseolus vulgaris, Phlogacanthus curviflorus, Photinia serratifolia, Physalis alkekengi, Picramnia antidesma, Picramnia hirsuta, Picramnia teapensis, Picramnia teapensis Tul., Picrasma quassioides, Pinalia stricta, Pisum sativum, Plantago coronopus, Plantago lanceolata, Pleione bulbocodioides, Pleione yunnanensis, Ploiarium alternifolium, Pluchea odora, Pneumatopteris pennigera, Polygoni cuspidati rhizoma et radix, Polygonum cuspidatum, Polygonum hypoleucum, Polygonum multiflorum, Polygonum perfoliatum, Pseudolarix amabilis, Psorospermum glaberrimum, Psorospermum laurentii, Psorospermum tenuifolium, Psychotria stachyoides, Quercus rubra, Radix Boehmeriae, Radix et Rhizoma Rhei, Radix et Rhizoma Rhei Praeparata, Ravenia spectabilis, Reynoutria japonica, Rhamnus cathartica, Rhamnus davurica, Rhamnus erythroxyloides, Rhamnus formosana, Rhamnus frangula [Syn. Frangula alnus], Rhamnus frangula, Rhamnus hirtella, Rhamnus kurdica, Rhamnus nakaharae, Rhamnus nepalensis, Rhamnus pallasii, Rhamnus prinoides, Rhamnus procumbens, Rhamnus punctata, Rhamnus saxatilis, Rhamnus thymifolius, Rhamnus triquetra, Rhamnus wightii, Rheum acuminatum, Rheum alexandrae, Rheum altaicum, Rheum australe, Rheum compactum, Rheum coreanum, Rheum delavayi, Rheum emodi [Syn. Rheum australe], Rheum franzenbachii, Rheum hotaoense, Rheum inopinatum, Rheum kialense, Rheum likiangense, Rheum nanum, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum spp., Rheum subacaule, Rheum sublanceolatum, Rheum tanguticum, Rheum tataricum, Rheum undulatum, Rheum uninerve, Rheum wittrockii, Rheum wittrocki, Rheum yunnanense, Rhizoma fagopyri cymose, Rhodiola viridula, Rhododendron dahuricum, Rosa pomifera, Rumex abyssinicus, Rumex acetosa, Rumex acetosella, Rumex aegyptiacus, Rumex alpinus, Rumex bequaertii, Rumex chalepensis, Rumex crispus, Rumex cristatus, Rumex cyprius, Rumex dentatus, Rumex japonicus, Rumex lanceolatus, Rumex limoniastrum, Rumex nepalensis, Rumex patientia, Rumex pictus, Rumex pulcher, Rumex ruwenzoriensis, Rumex scutatus, Rumex simpliciflorus, Rumex spp., Rumex tuberosus, Rumex usambarensis, Rumex vesicarius, Salvia aurea, Salvia greggii, Sargentodoxa cuneata, Sargentodoxae caulis, Saururus chinensis, Schistostephium artemisiifolium, Scilla socialis, Selaginella labordei, Semen pharbitidis, Senecio behnii, Senecio bonariensis, Senecio praecox, Senecio subdentatus, Senna alata, Senna alexandrina, Senna angustisiliqua, Senna artemisioides, Senna auriculata, Senna corymbosa, Senna didymobotrya, Sennae folium, Senna lindheimeriana, Senna macranthera, Senna obtusifolia, Senna occidentalis, Senna petersiana, Senna podocarpa, Senna septemtrionalis, Senna siamea, Senna sophera, Senna spectabilis, Senna tora, Simaba orinocensis, Smythea bombaiensis, Solanum cleistogamum, Sophorae tonkinensis radix et rhizome, Spatholobus suberectus, Sphaerella rosigena, Spinacia oleracea, Stevia berlandieri, Stevia ovata, Syzygium australe, Tagetes filifolia, Tanacetum coccineum, Taraxacum kok-saghyz, Taraxacum platycarpum subsp. hondoense, Tetradium ruticarpum, Teucrium yemense, Thymelaea hirsute, Tribuli fructus, Tribulus terrestris, Uvaria afzelii, Vatairea heteropteran, Ventilago calyculata, Ventilago denticulata, Ventilago leiocarpa, Ventilago madraspatana, Verbascum thapsiforme, Vismia guianensis, Vismia guineensis, Vismia laurentii, Vismia orientalis, Vitis vinifera, Wilbrandia ebracteata, Xanthii fructus, Xerochrysum viscosum, Angelica archangelica, Frangula purshiana, Geranium maculatum, Rumex crispus, Senecio congestus, Senna alexandrina, Aloe africana, Aloe arborescens, Aloe castellorum, Aloe elgonica, Aloe ferox, Aloe rubroviolacea, Aloe spicata, Aloe succotrina, Asphodeline tenuior, Asphodelus aestivus, Asphodelus albus, Asphodelus fistulosus, Asphodelus ramosus, Asphodelus tenuifolius, Berchemia floribunda, Bergenia hissarica, Bulbine annua, Bulbine asphodeloides, Callisia fragrans, Cassia aldabrensis, Cassia grandis, Cassia javanica, Chamaecrista mimosoides, Commiphora socotrana, Frangula alnus, Frangula purshiana, Kniphofia ensifolia, Kniphofia foliosa, Kniphofia insignis, Kniphofia isoetifolia, Kniphofia schimperi, Picramnia antidesma, Picramnia hirsute, Psidium guajava, Reynoutria japonica, Rhamnus alaternus, Rhamnus cathartica, Rhamnus davurica, Rheum acuminatum, Rheum alexandrae, Rheum altaicum, Rheum australe, Rheum compactum, Rheum coreanum, Rheum delavayi, Rheum franzenbachii, Rheum hotaoense, Rheum inopinatum, Rheum kialense, Rheum likiangense, Rheum nobile, Rheum palmatum, Rheum pumilum, Rheum racemiferum, Rheum reticulatum, Rheum rhabarbarum, Rheum rhaponticum, Rheum ribes, Rheum spiciforme, Rheum subacaule, Rheum tanguticum, Rheum tataricum, Rheum undulatum, Rheum uninerve, Rheum wittrockii, Rheum yunnanense, Rumex cristatus, Rumex paulsenianus, Rumex scutatus, Selaginella delicatula, Senna alata, Senna alexandrina, Senna didymobotrya, Senna obtusifolia, Senna occidentalis, Senna petersiana, Senna reticulata, Senna siamea, Senna tora, Spatholobus suberectus, Stephania dinklagei, Xanthorrhoea australis, Cichorium intybus, Cassia occidentalis, Morinda royoc, Galium aparine, Bryophyllum pinnatum, Anthema tinctoria, Aegle marmelos, Cymbopogon spp, Wedelia trilobata, Angelica archangelica, Vitis tilifolia, Mirabilis jalapa, Centipeda minima, Bauhinia manca, Juniperus drupacea, Forsythia suspensa, Citrus sinensis, Hylocomium splendens, Carthamus tinctorius, Rosmarinus officinalis, Oryza sativa, Trifolium pratense cv. Redman, Hordeum vulgare cv. Seco, Nepenthes x hookeriana, Musa acuminata, Pseudotsuga menziesii var. menziesii, Theobroma cacao, Hordeum vulgare cv. Seco, Nepenthes x hookeriana, Musa acuminata, Pseudotsuga menziesii var. menziesii, Penstemon centranthifolius, Pisum sativum, Glycine max, Avena sativa, Malus domestica, Gardenia jasminoides, Aloe ferox, Oryza sativa, Coffea arabica, Carthamus tinctorius, Macrotyloma uniflorum, Solanum melongena, Citrus unshiu, Hypericum perforatum, Curcuma longa, Nypa fruticans, Secale cereale cv. Aroostook, Persicaria tinctoria, Cannabis sativa, Solanum lycopersicum, Citrus clementina, Trifolium pratense cv. Redman, Uncaria tomentosa, Auricularia auricula-judae, Citrus sinensis, Cycas revoluta, Momordica charantia, Brassica carinata, Gaillardia pulchella, Nelumbo nucifera, Raphanus sativus var. niger, Lepidium meyenii, Zea mays, Nicotiana tabacum, Agaricus subrufescens, Linum usitatissimum, Paeonia lactiflora, Capsicum annuum, Larrea tridentata, Mirabilis jalapa, Catharanthus roseu, Medicago sativa subsp. sativa, Solanum americanum, Coffea arabica, Momordica charantia, Silybum marianum, Theobroma cacao, Aloe ferox, Gardenia jasminoides, Anacardium occidentale, Capsicum annuum, Paeonia lactiflora, Citrus clementina, Hordeum vulgare cv. Seco, Erucastrum gallicum, Linum usitatissimum, Asclepias syriaca, Avena sativa, Hypericum perforatum, Curcuma longa, Zea mays, Larrea tridentata, Nicotiana tabacum, Eucalyptus globulus subsp. bicostata, Musa acuminata, Solanum melongena, Phyllanthus emblica, Solanum lycopersicum, Veronica polita, Alcea rosea, Plukenetia volubilis, Euphorbia tirucalli, Neolamarckia cadamba, Citrus limon, Catharanthus roseus, Mirabilis jalapa, Podocarpus spicatus, Tripterygium wilfordii, Spatholobus suberectus, Crinum moorei, Juniperus monosperma, Pleurotus ostreatus, Gypsophila perfoliate, Clausena excavate, Gynura procumbens, Trifolium pratense cv. Redman, Oryza sativa, Rosmarinus officinalis, Nelumbo nucifera, Carthamus tinctorius, Citrus unshiu, Hylocomium splendens, Citrus sinensis, Juniperus drupacea, Forsythia suspensa, Momordica charantia, Silybum marianum, Aloe ferox, Citrus clementina, Hordeum vulgare cv. Seco, Erucastrum gallicum, Linum usitatissimum, Asclepias syriaca, Hypericum perforatum, Larrea tridentata, Eucalyptus globulus subsp. bicostata, Phyllanthus emblica, Veronica polita, Alcea rosea, Neolamarckia cadamba, Catharanthus roseus, Podocarpus spicatus, Tripterygium wilfordii, Juniperus monosperma, Pleurotus ostreatus, Gypsophila perfoliate, Clausena excavate, Gynura procumbens, Trifolium pratense cv. Redman, Rosmarinus officinalis, Nelumbo nucifera, Citrus unshiu, Hylocomium splendens, Citrus sinensis, Juniperus drupacea, Forsythia suspensa, or a combination thereof.

11. The use of claim 10, wherein the plant extract is selected from the group consisting of Rheum officinale, Theobroma cacao, Hordeum vulgare cv. Seco, Chaenomeles sinensis, Nepenthes x hookeriana, Musa acuminata, Pseudotsuga menziesii var. menziesii, Penstemon centranthifolius, Pisum sativum, Aloe Africana, Glycine max, Avena sativa, Malus domestica, Gardenia jasminoides, Aloe ferox, Oryza sativa, Coffea arabica, Carthamus tinctorius, Macrotyloma uniflorum, Solanum melongena, Aloe vera, Citrus unshiu, Hypericum perforatum, Curcuma longa, Nypa fruticans, Secale cereale cv. Aroostook, Persicaria tinctoria, Cannabis sativa, Solanum lycopersicum, Citrus clementina, Trifolium pratense cv. Redman, Uncaria tomentosa, Auricularia auricula-judae, Citrus sinensis, Cycas revoluta, Momordica charantia, Brassica carinata, Gaillardia pulchella, Nelumbo nucifera, Raphanus sativus var. niger, Lepidium meyenii, Zea mays, Nicotiana tabacum, Agaricus subrufescens, Linum usitatissimum, Paeonia lactiflora, Capsicum annuum, Larrea tridentata, Mirabilis jalapa, Catharanthus roseus, or a combination thereof.

12. The use of claim 10, wherein the plant extract is selected from the group consisting of Rheum officinale, Phyllanthus emblica, Theobroma cacao, or a combination thereof.

13. The use of claim 10, wherein the plant extract is selected from the group consisting of Tripterygium wilfordii, Phyllanthus emblica, Hypericum perforatum, Aloe barbadensis, Hypericum perforatum, or a combination thereof.

14. The use of claim 10, wherein the one or more compounds from the plant extract is selected from:

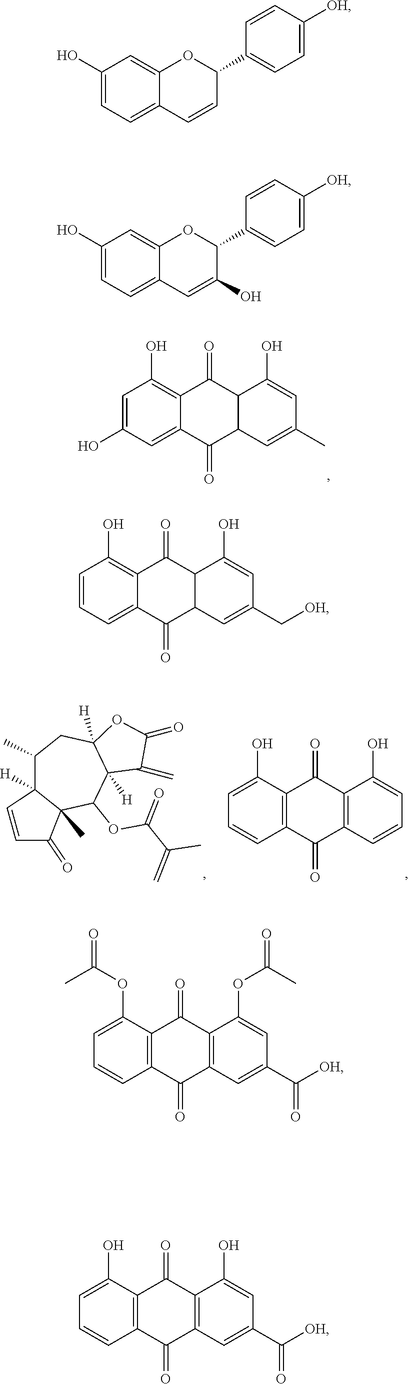

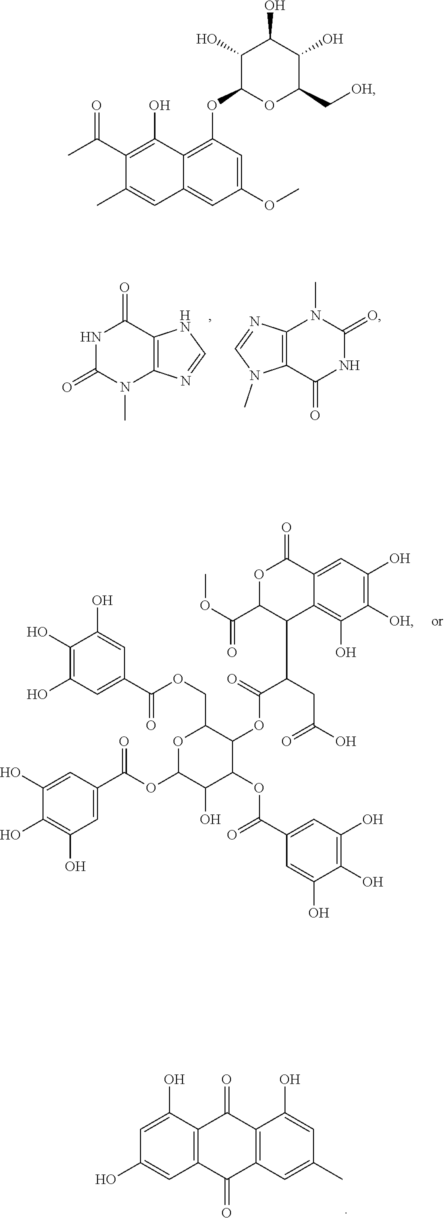

15. The use of claim 1, wherein the composition is formulated as a dietary supplement, a food ingredient or additive, a medical food, nutraceutical or pharmaceutical composition.

16. The use of claim 1, wherein the composition is in a dosage form configured as one of a soft-gel capsule, a hard capsule, gummy, functional beverage, functional food, liquid, powder, and a tablet.

17. The use of claim 1, wherein the composition comprises 1.0% to 99% w/w of the plant extract.

18. The use of claim 1, wherein the composition further comprises a preservative, a sweetener a coloring agent, an additive, a flavoring agent, or a combination thereof.

19. The use of claim 7, wherein the one or more compounds is

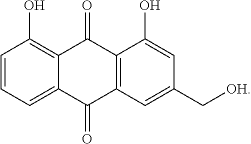 [SMILES: O=C1c2cccc(O)c2C(=O)c2c(O)cc(CO)cc21]

20. The use of claim 7, wherein the one or more compounds is

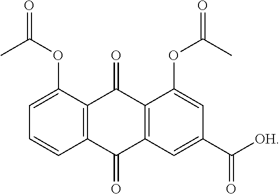 [SMILES: CC(=O)Oc1cccc2c1C(=O)c1c(OC(C)=O)cc(C(=O)O)cc1C2=O]

21. The use of claim 7, wherein the one or more compounds is

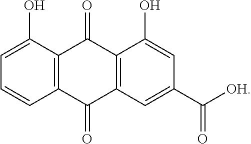 [SMILES: O=C(O)c1cc(O)c2c(c1)C(=O)c1cccc(O)c1C2=O]

22-50. (canceled)
